SEQUENCE LISTING (41 sequences)

SEQ ID NO: 1 - protein, 574 aa, Artificial Sequence
  Synthetic
MELLIHRSSAIFLTLAINTLYLTSSQNITEEFYQSTCSAVSRGYFSALRTGWYTSVITIELSNIKETKCNGTDTKVKLIKQELDKYKNAVTELQLLMQNTPAANNRARREAPQYMNYTINTTKNLNVSISKKRKRRFLGFLLGVGSAIASGIAVSKVLHLEGEVNKIKNALLSTNKAVVSLSNGVSVLTSKVLDLKNYINNQLLPIVNQQSCRISNIETVIEFQQKNSRLLEITREFSVNAGVTTPLSTYMLTNSELLSLINDMPITNDQKKLMSSNVQIVRQQSYSIMSIIKEEVLAYVVQLPIYGVIDTPCWKLHTSPLCTTNIKEGSNICLTRTDRGWYCDNAGSVSFFPQADTCKVQSNRVFCDTMNSLTLPSEVSLCNTDIFNSKYDCKIMTSKTDISSSVITSLGAIVSCYGKTKCTASNKNRGIIKTFSNGCDYVSNKGVDTVSVGNTLYYVNKLEGKNLYVKGEPIINYYDPLVFPSDEFDASISQVNEKINQSLAFIRRSDELLHNVNTGKSTTNIMITAIIIVIIVVLLSLIAIGLLLYCKAKNTPVTLSKDQLSGINNIAFSK

SEQ ID NO: 2 - RNA, 47 nt, Artificial Sequence
  Synthetic
gggaaauaagagagaaaagaagaguaagaagaaauauaagagccacc

SEQ ID NO: 3 - RNA, 57 nt, Artificial Sequence
  Synthetic
gggaaauaagagagaaaagaagaguaagaagaaauauaagaccccggcgccgccacc

SEQ ID NO: 4 - RNA, 119 nt, Artificial Sequence
  Synthetic
ugauaauaggcuggagccucgguggccaugcuucuugccccuugggccuccccccagccccuccuccccuuccugcacccguacccccguggucuuugaauaaagucugagugggcggc

SEQ ID NO: 5 - RNA, 119 nt, Artificial Sequence
  Synthetic
ugauaauaggcuggagccucgguggccuagcuucuugccccuugggccuccccccagccccuccuccccuuccugcacccguacccccguggucuuugaauaaagucugagugggcggc

SEQ ID NO: 6 - RNA, 1696 nt, Artificial Sequence
  Synthetic
gggaaauaagagagaaaagaagaguaagaagaaauauaagagccaccauggagcugcugauccugaaggccaacgccaucacgaccauccugaccgccgugaccuucugcuucgccagcgggcagaacaucaccgaggaguucuaccaguccaccugcuccgccgugagcaagggcuaccugucugcccugagaaccggcugguacaccagcgugaucaccaucgagcuguccaacaucaaggagaacaagugcaacggcaccgacgccaaggugaagcugaucaagcaggagcuggacaaguacaagaacgcagugaccgagcugcagcugcugaugcagagcacaccagccaccgguagcggguccgccauuugcuccggcguggccgugugcaaggugcugcaccuggagggcgaggugaacaagaucaagagcgcccugcucuccaccaacaaggccguggugagccugagcaacggggugagcgugcugaccuucaaggugcuggaccugaagaacuacaucgacaagcagcugcugccuauccugaacaagcagagcugcagcaucagcaacaucgagaccgugaucgaguuccagcagaagaacaaccggcugcuggagaucaccagggaguucagcgugaacgcaggggugaccacacccguguccaccuacaugcugaccaacuccgagcugcugagccugaucaacgauaugcccaucaccaacgaccagaagaagcugaugagcaacaacgugcagaucgugcggcagcaguccuacuccaucaugugcaucaucaaggaggaggugcuggccuacguggugcagcugccccuguacggcgugaucgacaccccuugcuggaagcugcacaccagcccucugugcaccaccaacacgaaggagggcagcaauaucugccugacccggaccgacaggggcugguacugcgacaacgccggcagcguguccuucuuuccccaggccgagaccugcaaggugcaguccaacaggguguucugcgacaccaugaacucucgcacccugcccagcgaggugaaccugugcaacguggacaucuucaaccccaaguacgacugcaagaucaugaccuccaagaccgacguguccucuagcguuaucaccucccugggcgccaucgugagcugcuacggcaagaccaagugcaccgccagcaacaagaacaggggcaucaucaagaccuucagcaacgggugcgacuacguguccaacaagggcguggacaccguguccgugggcaacacccuguacugcgugaacaagcaggagggcaagagccuguacgugaagggcgagcccaucaucaacuucuacgacccucugguguuccccagcgacgaguucgacgccagcaucucccaggugaacgagaagaucaaccagagccuggccuucauccgcaagagcgacgagcugcugcacaacgugaacgccggcaagagcaccacaaacaucaugaucaccaccaucaucaucgugauaaucgugauccugcugucccugaucgcugugggccugcugcuguacugauaauaggcuggagccucgguggccaugcuucuugccccuugggccuccccccagccccuccuccccuuccugcacccguacccccguggucuuugaauaaagucugagugggcggc

SEQ ID NO: 7 - RNA, 1530 nt, Artificial Sequence
  Synthetic
auggagcugcugauccugaaggccaacgccaucacgaccauccugaccgccgugaccuucugcuucgccagcgggcagaacaucaccgaggaguucuaccaguccaccugcuccgccgugagcaagggcuaccugucugcccugagaaccggcugguacaccagcgugaucaccaucgagcuguccaacaucaaggagaacaagugcaacggcaccgacgccaaggugaagcugaucaagcaggagcuggacaaguacaagaacgcagugaccgagcugcagcugcugaugcagagcacaccagccaccgguagcggguccgccauuugcuccggcguggccgugugcaaggugcugcaccuggagggcgaggugaacaagaucaagagcgcccugcucuccaccaacaaggccguggugagccugagcaacggggugagcgugcugaccuucaaggugcuggaccugaagaacuacaucgacaagcagcugcugccuauccugaacaagcagagcugcagcaucagcaacaucgagaccgugaucgaguuccagcagaagaacaaccggcugcuggagaucaccagggaguucagcgugaacgcaggggugaccacacccguguccaccuacaugcugaccaacuccgagcugcugagccugaucaacgauaugcccaucaccaacgaccagaagaagcugaugagcaacaacgugcagaucgugcggcagcaguccuacuccaucaugugcaucaucaaggaggaggugcuggccuacguggugcagcugccccuguacggcgugaucgacaccccuugcuggaagcugcacaccagcccucugugcaccaccaacacgaaggagggcagcaauaucugccugacccggaccgacaggggcugguacugcgacaacgccggcagcguguccuucuuuccccaggccgagaccugcaaggugcaguccaacaggguguucugcgacaccaugaacucucgcacccugcccagcgaggugaaccugugcaacguggacaucuucaaccccaaguacgacugcaagaucaugaccuccaagaccgacguguccucuagcguuaucaccucccugggcgccaucgugagcugcuacggcaagaccaagugcaccgccagcaacaagaacaggggcaucaucaagaccuucagcaacgggugcgacuacguguccaacaagggcguggacaccguguccgugggcaacacccuguacugcgugaacaagcaggagggcaagagccuguacgugaagggcgagcccaucaucaacuucuacgacccucugguguuccccagcgacgaguucgacgccagcaucucccaggugaacgagaagaucaaccagagccuggccuucauccgcaagagcgacgagcugcugcacaacgugaacgccggcaagagcaccacaaacaucaugaucaccaccaucaucaucgugauaaucgugauccugcugucccugaucgcugugggccugcugcuguac

SEQ ID NO: 8 - protein, 510 aa, Artificial Sequence
  Synthetic
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATGSGSAICSGVAVCKVLHLEGEVNKIKSALLSTNKAVVSLSNGVSVLTFKVLDLKNYIDKQLLPILNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSRTLPSEVNLCNVDIFNPKYDCKIMTSKTDVSSSVITSLGAIVSCYGKTKCTASNKNRGIIKTFSNGCDYVSNKGVDTVSVGNTLYCVNKQEGKSLYVKGEPIINFYDPLVFPSDEFDASISQVNEKINQSLAFIRKSDELLHNVNAGKSTTNIMITTIIIVIIVILLSLIAVGLLLY

SEQ ID NO: 9 - RNA, 1786 nt, Artificial Sequence
  Synthetic
gggaaauaagagagaaaagaagaguaagaagaaauauaagagccaccaugagcuggaagguggugauuaucuucagccugcugauuacaccucaacacggccugaaggagagcuaccuggaagagagcugcuccaccaucaccgagggcuaccugagcgugcugcggaccggcugguacaccaacguguucacccuggaggugggcgacguggagaaccugaccugcagcgacggcccuagccugaucaagaccgagcuggaccugaccaagagcgcucugagagagcugaagaccguguccgccgaccagcuggccagagaggaacagaucgagaacccucggcagagcagauucgugcugggcgccaucgcucugggagucgccgcugccgcugcagugacagcuggaguggccauugcuaagaccaucagacuggaaagcgaggugacagccaucaacaaugcccugaagaagaccaacgaggccgugagcacccugggcaauggagugagagugcuggccacagccgugcgggagcugaaggacuucgugagcaagaaccugaccagagccaucaacaagaacaagugcgacaucgaugaccugaagauggccgugagcuucucccaguucaacagacgguuccugaacguggugagacaguucuccgacaacgcuggaaucacaccugccauuagccuggaccugaugaccgacgccgagcuggcuagagccgugcccaacaugcccaccagcgcuggccagaucaagcugaugcuggagaacagagccauggugcggagaaagggcuucggcauccugauugggguguauggaagcuccgugaucuacauggugcagcugcccaucuucggcgugaucgacacacccugcuggaucgugaaggccgcuccuagcugcuccgagaagaaaggaaacuaugccugucugcugagagaggaccagggcugguacugccagaacgccggaagcacaguguacuaucccaacgagaaggacugcgagaccagaggcgaccacguguucugcgacaccgcugccggaaucaacguggccgagcagagcaaggagugcaacaucaacaucagcacaaccaacuaccccugcaaggugagcaccggacggcaccccaucagcaugguggcucugagcccucugggcgcucugguggccugcuauaagggcguguccuguagcaucggcagcaaucgggugggcaucaucaagcagcugaacaagggaugcuccuacaucaccaaccaggacgccgacaccgugaccaucgacaacaccguguaccagcugagcaagguggagggcgagcagcacgugaucaagggcagacccgugagcuccagcuucgaccccaucaaguucccugaggaccaguucaacguggcccuggaccagguguuugagaacaucgagaacagccaggcccugguggaccagagcaacagaauccuguccagcgcugagaagggcaacaccggcuucaucauugugaucauucugaucgccgugcugggcagcuccaugauccuggugagcaucuucaucauuaucaagaagaccaagaaacccaccggagccccuccugagcugagcggcgugaccaacaauggcuucauuccccacaacugaugauaauaggcuggagccucgguggccaugcuucuugccccuugggccuccccccagccccuccuccccuuccugcacccguacccccguggucuuugaauaaagucugagugggcggc

SEQ ID NO: 10 - RNA, 1620 nt, Artificial Sequence
  Synthetic
augagcuggaagguggugauuaucuucagccugcugauuacaccucaacacggccugaaggagagcuaccuggaagagagcugcuccaccaucaccgagggcuaccugagcgugcugcggaccggcugguacaccaacguguucacccuggaggugggcgacguggagaaccugaccugcagcgacggcccuagccugaucaagaccgagcuggaccugaccaagagcgcucugagagagcugaagaccguguccgccgaccagcuggccagagaggaacagaucgagaacccucggcagagcagauucgugcugggcgccaucgcucugggagucgccgcugccgcugcagugacagcuggaguggccauugcuaagaccaucagacuggaaagcgaggugacagccaucaacaaugcccugaagaagaccaacgaggccgugagcacccugggcaauggagugagagugcuggccacagccgugcgggagcugaaggacuucgugagcaagaaccugaccagagccaucaacaagaacaagugcgacaucgaugaccugaagauggccgugagcuucucccaguucaacagacgguuccugaacguggugagacaguucuccgacaacgcuggaaucacaccugccauuagccuggaccugaugaccgacgccgagcuggcuagagccgugcccaacaugcccaccagcgcuggccagaucaagcugaugcuggagaacagagccauggugcggagaaagggcuucggcauccugauugggguguauggaagcuccgugaucuacauggugcagcugcccaucuucggcgugaucgacacacccugcuggaucgugaaggccgcuccuagcugcuccgagaagaaaggaaacuaugccugucugcugagagaggaccagggcugguacugccagaacgccggaagcacaguguacuaucccaacgagaaggacugcgagaccagaggcgaccacguguucugcgacaccgcugccggaaucaacguggccgagcagagcaaggagugcaacaucaacaucagcacaaccaacuaccccugcaaggugagcaccggacggcaccccaucagcaugguggcucugagcccucugggcgcucugguggccugcuauaagggcguguccuguagcaucggcagcaaucgggugggcaucaucaagcagcugaacaagggaugcuccuacaucaccaaccaggacgccgacaccgugaccaucgacaacaccguguaccagcugagcaagguggagggcgagcagcacgugaucaagggcagacccgugagcuccagcuucgaccccaucaaguucccugaggaccaguucaacguggcccuggaccagguguuugagaacaucgagaacagccaggcccugguggaccagagcaacagaauccuguccagcgcugagaagggcaacaccggcuucaucauugugaucauucugaucgccgugcugggcagcuccaugauccuggugagcaucuucaucauuaucaagaagaccaagaaacccaccggagccccuccugagcugagcggcgugaccaacaauggcuucauuccccacaacuga

SEQ ID NO: 11 - protein, 541 aa, Artificial Sequence
  Synthetic
MSWKVVIIFSLLITPQHGLKESYLEESCSTITEGYLSVLRTGWYTNVFTLEVGDVENLTCSDGPSLIKTELDLTKSALRELKTVSADQLAREEQIENPRQSRFVLGAIALGVAAAAAVTAGVAIAKTIRLESEVTAINNALKKTNEAVSTLGNGVRVLATAVRELKDFVSKNLTRAINKNKCDIDDLKMAVSFSQFNRRFLNVVRQFSDNAGITPAISLDLMTDAELARAVPNMPTSAGQIKLMLENRAMETVRRKGFGILIGVYGSSVIYMVQLPIFGVIDTPCWIVKAAPSCSEKKGNYACLLREDQGWYCQNAGSTVYYPNEKDCETRGDHVFCDTAAGINVAEQSKECNINISTTNYPCKVSTGRHPISMVALSPLGALVACYKGVSCSIGSNRVGIIKQLNKGCSYITNQDADTVTIDNTVYQLSKVEGEQHVIKGRPVSSSFDPIKFPEDQFNVALDQVFENIENSQALVDQSNRILSSAEKGNTGFIIVIILIAVLGSSMILVSIFIIIKKTKKPTGAPPELSGVTNNGFIPHN

SEQ ID NO: 12 - RNA, 1783 nt, Artificial Sequence
  Synthetic
gggaaauaagagagaaaagaagaguaagaagaaauauaagagccaccaugcccaucagcauccugcugaucaucaccacaaugaucauggccagccacugccagaucgacaucaccaagcugcagcacgugggcgugcucgugaacagccccaagggcaugaagaucagccagaacuucgagacacgcuaccugauccugagccugauccccaagaucgaggacagcaacagcugcggcgaccagcagaucaagcaguacaagcggcugcuggacagacugaucaucccccuguacgacggccugcggcugcagaaagacgugaucgugaccaaccaggaaagcaacgagaacaccgacccccggaccgagagauucuucggcggcgugaucggcacaaucgcccugggaguggccacaagcgcccagauuacagccgcuguggcccugguggaagccaagcaggccagaagcgacaucgagaagcugaaagaggccauccgggacaccaacaaggccgugcagagcgugcaguccagcgugggcaaucugaucguggccaucaaguccgugcaggacuacgugaacaaagaaaucgugcccucuaucgcccggcugggcugugaagcugccggacugcagcugggcauugcccugacacagcacuacagcgagcugaccaacaucuucggcgacaacaucggcagccugcaggaaaagggcauuaagcugcagggaaucgccagccuguaccgcaccaacaucaccgagaucuucaccaccagcaccguggauaaguacgacaucuacgaccugcuguucaccgagagcaucaaagugcgcgugaucgacguggaccugaacgacuacagcaucacccugcaagugcggcugccccugcugaccagacugcugaacacccagaucuacaagguggacagcaucuccuacaacauccagaaccgcgagugguacaucccucugcccagccacauuaugaccaagggcgccuuucugggcggagccgacgugaaagagugcaucgaggccuucagcagcuacaucugccccagcgacccuggcuucgugcugaaccacgagauggaaagcugccugagcggcaacaucagccagugccccagaaccaccgugaccuccgacaucgugcccagauacgccuucgugaauggcggcgugguggccaacugcaucaccaccaccuguaccugcaacggcaucggcaaccggaucaaccagccucccgaucagggcgugaagauuaucacccacaaagaguguaacaccaucggcaucaacggcaugcuguucaauaccaacaaagagggcacccuggccuucuacacccccgacgauaucacccugaacaacuccguggcucuggaccccaucgacaucuccaucgagcugaacaaggccaagagcgaccuggaagaguccaaagaguggauccggcggagcaaccagaagcuggacucuaucggcagcuggcaccagagcagcaccaccaucaucgugauccugauuaugaugauuauccuguucaucaucaacauuaccaucaucacuaucgccauuaaguacuaccggauccagaaacggaaccggguggaccagaaugacaagcccuacgugcugacaaacaagugauaauaggcuggagccucgguggccaugcuucuugccccuugggccuccccccagccccuccuccccuuccugcacccguacccccguggucuuugaauaaagucugagugggcggc

SEQ ID NO: 13 - RNA, 1617 nt, Artificial Sequence
  Synthetic
augcccaucagcauccugcugaucaucaccacaaugaucauggccagccacugccagaucgacaucaccaagcugcagcacgugggcgugcucgugaacagccccaagggcaugaagaucagccagaacuucgagacacgcuaccugauccugagccugauccccaagaucgaggacagcaacagcugcggcgaccagcagaucaagcaguacaagcggcugcuggacagacugaucaucccccuguacgacggccugcggcugcagaaagacgugaucgugaccaaccaggaaagcaacgagaacaccgacccccggaccgagagauucuucggcggcgugaucggcacaaucgcccugggaguggccacaagcgcccagauuacagccgcuguggcccugguggaagccaagcaggccagaagcgacaucgagaagcugaaagaggccauccgggacaccaacaaggccgugcagagcgugcaguccagcgugggcaaucugaucguggccaucaaguccgugcaggacuacgugaacaaagaaaucgugcccucuaucgcccggcugggcugugaagcugccggacugcagcugggcauugcccugacacagcacuacagcgagcugaccaacaucuucggcgacaacaucggcagccugcaggaaaagggcauuaagcugcagggaaucgccagccuguaccgcaccaacaucaccgagaucuucaccaccagcaccguggauaaguacgacaucuacgaccugcuguucaccgagagcaucaaagugcgcgugaucgacguggaccugaacgacuacagcaucacccugcaagugcggcugccccugcugaccagacugcugaacacccagaucuacaagguggacagcaucuccuacaacauccagaaccgcgagugguacaucccucugcccagccacauuaugaccaagggcgccuuucugggcggagccgacgugaaagagugcaucgaggccuucagcagcuacaucugccccagcgacccuggcuucgugcugaaccacgagauggaaagcugccugagcggcaacaucagccagugccccagaaccaccgugaccuccgacaucgugcccagauacgccuucgugaauggcggcgugguggccaacugcaucaccaccaccuguaccugcaacggcaucggcaaccggaucaaccagccucccgaucagggcgugaagauuaucacccacaaagaguguaacaccaucggcaucaacggcaugcuguucaauaccaacaaagagggcacccuggccuucuacacccccgacgauaucacccugaacaacuccguggcucuggaccccaucgacaucuccaucgagcugaacaaggccaagagcgaccuggaagaguccaaagaguggauccggcggagcaaccagaagcuggacucuaucggcagcuggcaccagagcagcaccaccaucaucgugauccugauuaugaugauuauccuguucaucaucaacauuaccaucaucacuaucgccauuaaguacuaccggauccagaaacggaaccggguggaccagaaugacaagcccuacgugcugacaaacaag

SEQ ID NO: 14 - protein, 539 aa, Artificial Sequence
  Synthetic
MPISILLIITTMIMASHCQIDITKLQHVGVLVNSPKGMKISQNFETRYLILSLIPKIEDSNSCGDQQIKQYKRLLDRLIIPLYDGLRLQKDVIVTNQESNENTDPRTERFFGGVIGTIALGVATSAQITAAVALVEAKQARSDIEKLKEAIRDTNKAVQSVQSSVGNLIVAIKSVQDYVNKEIVPSIARLGCEAAGLQLGIALTQHYSELTNIFGDNIGSLQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALDPIDISIELNKAKSDLEESKEWIRRSNQKLDSIGSWHQSSTTIIVILIMMIILFIINITIITIAIKYYRIQKRNRVDQNDKPYVLTNK

SEQ ID NO: 15 - RNA, 1696 nt, Artificial Sequence; identical to SEQ ID NO: 6
  Synthetic

SEQ ID NO: 16 - RNA, 1786 nt, Artificial Sequence; identical to SEQ ID NO: 9
  Synthetic

SEQ ID NO: 17 - RNA, 1783 nt, Artificial Sequence; identical to SEQ ID NO: 12
  Synthetic

SEQ ID NO: 18 - protein, 30 aa, Artificial Sequence
  Synthetic
MDSKGSSQKGSRLLLLLVVSNLLLPQGVVG

SEQ ID NO: 19 - protein, 18 aa, Artificial Sequence
  Synthetic
MDWTWILFLVAAATRVHS

SEQ ID NO: 20 - protein, 20 aa, Artificial Sequence
  Synthetic
METPAQLLFLLLLWLPDTTG

SEQ ID NO: 21 - protein, 24 aa, Artificial Sequence
  Synthetic
MLGSNSGQRVVFTILLLLVAPAYS

SEQ ID NO: 22 - protein, 17 aa, Artificial Sequence
  Synthetic
MKCLLYLAFLFIGVNCA

SEQ ID NO: 23 - protein, 15 aa, Artificial Sequence
  Synthetic
MWLVSLAIVTACAGA

SEQ ID NO: 24 - skipped

SEQ ID NO: 25 - protein, 25 aa, Artificial Sequence
  Synthetic
CKARSTPVTLSKDQLSGINNIAFSN

SEQ ID NO: 26 - protein, 20 aa, Artificial Sequence
  Synthetic
TPVTLSKDQLSGINNIAFSN

SEQ ID NO: 27 - protein, 15 aa, Artificial Sequence
  Synthetic
SKDQLSGINNIAFSN

SEQ ID NO: 28 - protein, 10 aa, Artificial Sequence
  Synthetic
SGINNIAFSN

SEQ ID NO: 29 - RNA, 9 nt, Artificial Sequence
  Synthetic
  misc_feature (3)..(9): may be Adenine or Guanine
ccrccaugg

SEQ ID NO: 30 - RNA, 11 nt, Artificial Sequence
  Synthetic
gggauccuacc

SEQ ID NO: 31 - RNA, 9 nt, Artificial Sequence
  Synthetic
  misc_feature (8)..(9): N may be Adenine or Uracil
uuauuuann

SEQ ID NO: 32 - RNA, 1696 nt, Artificial Sequence; identical to SEQ ID NO: 6
  Synthetic
  misc_feature (1)..(1696): is modified by N1-methylpseudoruidine

SEQ ID NO: 33 - RNA, 47 nt, Artificial Sequence; identical to SEQ ID NO: 2
  Synthetic
  misc_feature (1)..(47): is modified by N1-methylpseudoruidine

SEQ ID NO: 34 - RNA, 1530 nt, Artificial Sequence; identical to SEQ ID NO: 7
  Synthetic
  misc_feature (1)..(1530): is modified by N1-methylpseudoruidine

SEQ ID NO: 35 - RNA, 119 nt, Artificial Sequence; identical to SEQ ID NO: 4
  Synthetic
  misc_feature (1)..(119): is modified by N1-methylpseudoruidine

SEQ ID NO: 36 - RNA, 1786 nt, Artificial Sequence; identical to SEQ ID NO: 9
  Synthetic
  misc_feature (1)..(1786): is modified by N1-methylpseudoruidine

SEQ ID NO: 37 - RNA, 1620 nt, Artificial Sequence; identical to SEQ ID NO: 10
  Synthetic
  misc_feature (1)..(1620): is modified by N1-methylpseudoruidine

SEQ ID NO: 38 - RNA, 1783 nt, Artificial Sequence; identical to SEQ ID NO: 12
  Synthetic
  misc_feature (1)..(1783): is modified by N1-methylpseudoruidine

SEQ ID NO: 39 - RNA, 1617 nt, Artificial Sequence; identical to SEQ ID NO: 13
  Synthetic
  misc_feature (1)..(1617): is modified by N1-methylpseudoruidine

SEQ ID NO: 40 - RNA, 57 nt, Artificial Sequence; identical to SEQ ID NO: 3
  Synthetic
  misc_feature (1)..(57): is modified by N1-methylpseudoruidine

SEQ ID NO: 41 - RNA, 119 nt, Artificial Sequence; identical to SEQ ID NO: 5
  Synthetic
  misc_feature (1)..(119): is modified by N1-methylpseudoruidine

(19) United States
(12) Patent Application Publication
(10) Pub. No.: US 2023/0338506 A1
(43) Pub. Date: Oct. 26, 2023

(54) RESPIRATORY VIRUS IMMUNIZING COMPOSITIONS

(71) Applicant: ModernaTX, Inc., Cambridge, MA (US)

(72) Inventors: Christine Shaw, Cambridge, MA (US); Guillaume Stewart-Jones, Cambridge, MA (US); Elisabeth Narayanan, Cambridge, MA (US); Vladimir Presnyak, Manchester, NH (US); Sayda Mahgoub Elbashir, Cambridge, MA (US)

(73) Assignee: ModernaTX, Inc., Cambridge, MA (US)

(21) Appl. No.: 17/796,401

(22) PCT Filed: Jan. 29, 2021
(86) PCT No.: PCT/US2021/015840

§ 371 Date: Jul. 29, 2022

Related U.S. Application Data
(60) Provisional application No. 62/967,888, filed on Jan. 30, 2020.

(51) Int. Cl. A61K 39/275; A61K 9/127; C12N 15/86; A61K 39/155; A61P 31/14; C07K 14/005

(52) U.S. Cl. CPC A61K 39/155; A61K 9/1271; A61K 39/275; A61P 31/14; C07K 14/005; C12N 15/86; A61K 2039/53

(57) ABSTRACT

The disclosure relates to respiratory virus ribonucleic acid (RNA) vaccines as well as methods of using the vaccines and compositions comprising the vaccines.

### RELATED APPLICATION

This application claims the benefit under 35 U.S.C. 119(e) of the filing date of U.S. Provisional Application Serial Number 62/967,888, filed Jan. 30, 2020, the entire contents of which is incorporated herein by reference.

### BACKGROUND

Respiratory disease is a medical term that encompasses pathological conditions affecting the organs and tissues that make gas exchange possible in higher organisms, and includes conditions of the upper respiratory tract, trachea, bronchi, bronchioles, alveoli, pleura and pleural cavity, and the nerves and muscles of breathing. Respiratory diseases range from mild and self-limiting, such as the common cold, to life-threatening entities like bacterial pneumonia, pulmonary embolism, acute asthma and lung cancer. Respiratory disease is a common and significant cause of illness and death around the world. In the US, approximately 1 billion “common colds” occur each year. Respiratory conditions are among the most frequent reasons for hospital stays among children.

Human respiratory syncytial virus (hRSV) is a negative-sense, single-stranded ribonucleic acid (RNA) virus of the genus Pneumovirinae. The virus is present in at least two antigenic subgroups, known as Group A and Group B, primarily resulting from differences in the surface G glycoproteins. Two hRSV surface glycoproteins - G and F - mediate attachment with and attachment to cells of the respiratory epithelium. F surface glycoproteins mediate coalescence of neighboring cells. This results in the formation of syncytial cells. hRSV is the most common cause of bronchiolitis. Most infected adults develop mild cold-like symptoms such as congestion, low-grade fever, and wheezing. Infants and small children may suffer more severe symptoms such as bronchiolitis and pneumonia. The disease may be transmitted among humans via contact with respiratory secretions.

Human metapneumovirus (hMPV) is a negative-sense, single-stranded RNA virus of the genus Pneumovirinae and of the family Paramyxoviridae and is closely related to the avian metapneumovirus (AMPV) subgroup C. It was isolated for the first time in 2001 in the Netherlands by using the RAP-PCR (RNA arbitrarily primed PCR) technique for identification of unknown viruses growing in cultured cells. hMPV is second only to hRSV as an important cause of viral lower respiratory tract illness (LRI) in young children. The seasonal epidemiology of hMPV appears to be similar to that of hRSV, but the incidence of infection and illness appears to be substantially lower.

Parainfluenza virus type 3 (PIV3), like hMPV, is also a negative-sense, single-stranded sense RNA virus of the genus Pneumovirinae and of the family Paramyxoviridae and is a major cause of ubiquitous acute respiratory infections of infancy and early childhood. Its incidence peaks around 4-12 months of age, and the virus is responsible for 3-10% of hospitalizations, mainly for bronchiolitis and pneumonia. PIV3 can be fatal, and in some instances is associated with neurologic diseases, such as febrile seizures. It can also result in airway remodeling, a significant cause of morbidity. In developing regions of the world, infants and young children are at the highest risk of mortality, either from primary PIV3 viral infection or from secondary consequences, such as bacterial infections. Human parainfluenza viruses (hPIV) types 1, 2 and 3 (hPIV1, hPIV2 and hPIV3, respectively), also like hMPV, are second only to hRSV as important causes of viral LRI in young children.

The continuing health problems associated with hMPV, hPIV3 and hRSV are of concern internationally, reinforcing the importance of developing effective and safe vaccine candidates against these viruses.

### SUMMARY

Provided herein, in some embodiments, are immunizing compositions (e.g., RNA vaccines and other immunogenic compositions) that comprise an RNA that encodes highly immunogenic antigens capable of eliciting potent neutralizing antibodies responses against respiratory virus antigens, such as human respiratory syncytial virus (hRSV) antigens, human metapneumovirus (hMPV) antigens, and/or human parainfluenza virus 3 (hPIV3) antigens. Surprisingly, the data provided herein show that immunizing compositions that comprise an hRSV RNA encoding a stabilized prefusion form of an hRSV F glycoprotein that lacks a cytoplasmic tail, when administered to animals, induces a highly neutralizing antibody response against hRSV F glycoprotein, even at doses that are approximately 5-fold lower than control compositions.

Some aspects of the present disclosure provide a composition (e.g., immunizing, immunogenic, and/or vaccine composition) comprising a human respiratory syncytial virus (hRSV) ribonucleic acid (RNA) encoding a stabilized prefusion form of an hRSV F glycoprotein variant that lacks a cytoplasmic tail and has at least 90% (e.g., at least 91%, at least 92%, at least 93%, at least 94%, at least 95%, at least 96%, at least 97%, at least 98%, at least 99%, or 100%) identity to a wild-type hRSV F glycoprotein.

Other aspects of the present disclosure provide a human respiratory syncytial virus (hRSV) ribonucleic acid (RNA) encoding a stabilized prefusion form of an RSV F glycoprotein variant that lacks a cytoplasmic tail, wherein the RSV F glycoprotein variant has at least 85% identity to a full-length wild-type RSV F glycoprotein; a human metapneumovirus (hMPV) RNA encoding an hMPV F glycoprotein; and a human parainfluenza virus 3 (hPIV3) RNA encoding an hPIV3 F glycoprotein.

Yet other aspects of the present disclosure provide a messenger ribonucleic acid (mRNA) comprising an open reading frame that comprises a sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the sequence of SEQ ID NO: 7. Further aspects of the present disclosure provide a messenger ribonucleic acid (mRNA) comprising a sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the sequence of SEQ ID NO: 15. In some embodiments, the mRNA encodes a stabilized prefusion form of an human respiratory syncytial virus (hRSV) ribonucleic F glycoprotein variant that lacks a cytoplasmic tail, wherein the RSV F glycoprotein variant has at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a full-length wild-type RSV F glycoprotein. In some embodiments, the open reading from comprises the sequence of SEQ ID NO: 7. In some embodiments, the mRNA is formulated in a lipid nanoparticle.

In some embodiments, the cytoplasmic tail comprises the C-terminal 20-30, 20-25, 15-30, 15-25, 15-20, 10-30, 10-25, 10-20, 10-15, 5-30, 5-25, 5-20, or 5-15 amino acids of the of the hRSV F glycoprotein variant. In some embodiments, the cytoplasmic tail comprises the C-terminal 25 amino acids, 20 amino acids, 15 amino acids, or 10 amino acids of the hRSV F glycoprotein variant. In some embodiments, the cytoplasmic tail comprises of the following C-terminal amino acids of the hRSV F glycoprotein variant: CKARSTPVTLSKDQLSGINNIAFSN (SEQ ID NO: 25); TPVTLSKDQLSGINNIAFSN (SEQ ID NO: 26); SKDQLSGINNIAFSN (SEQ ID NO: 27); or SGINNIAFSN (SEQ ID NO: 28).

In some embodiments, the hRSV F glycoprotein variant further comprises a modification, relative to the wild-type hRSV F glycoprotein, selected from the group consisting of: a P102X substitution, a substitution of amino acids 104-144 with a linker molecule, an A149X substitution, an S155X substitution, an S190X substitution, a V207X substitution, an S290X substitution, a L373X substitution, an I379X substitution, an M447X substitution, and a Y458X substitution, wherein X is any amino acid.

In some embodiments, the hRSV F glycoprotein variant further comprises a modification, relative to the wild-type hRSV F glycoprotein, selected from the group consisting of: a P102A substitution, a substitution of amino acids 104-144 with a linker molecule, an A149C substitution, an S155C substitution, an S190F substitution, a V207L substitution, an S290C substitution, a L373R substitution, an I379V substitution, an M447V substitution, and a Y458C substitution.

In some embodiments, the hRSV F glycoprotein variant further comprises the following modifications, relative to the wild-type hRSV F glycoprotein: a P102A substitution, a substitution of amino acids 104-144 with a linker molecule, an A149C substitution, an S155C substitution, an S190F substitution, a V207L substitution, an S290C substitution, a L373R substitution, an I379V substitution, an M447V substitution, and a Y458C substitution.

In some embodiments, the wild-type hRSV F glycoprotein comprises the sequence of SEQ ID NO: 1.

In some embodiments, the hRSV F glycoprotein variant comprises a sequence that has at least 95% or at least 98% identity to the sequence of SEQ ID NO: 8. In some embodiments, the hRSV F glycoprotein variant comprises the sequence of SEQ ID NO: 8.

In some embodiments, the hRSV RNA comprises an open reading frame (ORF) that comprises a sequence that has at least 90%, at least 95%, or at least 98% identity to the sequence of SEQ ID NO: 7. In some embodiments, the hRSV RNA comprises an ORF that comprises the sequence of SEQ ID NO: 7.

In some embodiments, the hRSV RNA comprises a 5′ untranslated region (UTR) that comprises the sequence of SEQ ID NO: 2. In some embodiments, the hRSV RNA comprises a 3′ UTR that comprises the sequence of SEQ ID NO: 4.

In some embodiments, the hRSV RNA comprises a sequence that has at least 90%, at least 91%, at least 92%, at least 93%, at least 94%, at least 95%, at least 96%, at least 97%, at least 98%, at least 99%, or 100% identity to the sequence of SEQ ID NO: 15. In some embodiments, the hRSV RNA comprises the sequence of SEQ ID NO: 15.

In some embodiments, the hMPV F glycoprotein comprises a sequence that has at least 90%, at least 91%, at least 92%, at least 93%, at least 94%, at least 95%, at least 96%, at least 97%, at least 98%, at least 99%, or 100% identity to the sequence of SEQ ID NO: 11. In some embodiments, the hMPV F glycoprotein comprises the sequence of SEQ ID NO: 11.

In some embodiments, the hMPV RNA comprises an ORF that comprises a sequence that has at least 90%, at least 91%, at least 92%, at least 93%, at least 94%, at least 95%, at least 96%, at least 97%, at least 98%, at least 99%, or 100% identity to the sequence of SEQ ID NO: 10. In some embodiments, the hMPV RNA comprises an ORF that comprises the sequence of SEQ ID NO: 10.

In some embodiments, the hMPV RNA comprises a 5′ UTR that comprises the sequence of SEQ ID NO: 2. In some embodiments, the hMPV RNA comprises a 3′ UTR that comprises the sequence of SEQ ID NO: 4.

In some embodiments, the hMPV RNA comprises a sequence that has at least 90%, at least 91%, at least 92%, at least 93%, at least 94%, at least 95%, at least 96%, at least 97%, at least 98%, at least 99%, or 100% identity to the sequence of SEQ ID NO: 16. In some embodiments, the hMPV RNA comprises the sequence of SEQ ID NO: 16.

In some embodiments, the hPIV3 F glycoprotein comprises a sequence that has at least 90%, at least 91%, at least 92%, at least 93%, at least 94%, at least 95%, at least 96%, at least 97%, at least 98%, at least 99%, or 100% identity to the sequence of SEQ ID NO: 14. In some embodiments, the hPIV3 F glycoprotein comprises the sequence of SEQ ID NO: 14.

n some embodiments, the hPIV3 RNA comprises an ORF that comprises a sequence that has at least 90%, at least 91%, at least 92%, at least 93%, at least 94%, at least 95%, at least 96%, at least 97%, at least 98%, at least 99%, or 100% identity to the sequence of SEQ ID NO: 13. In some embodiments, the hPIV3 RNA comprises an ORF that comprises the sequence of SEQ ID NO: 13.

In some embodiments, the hPIV3 RNA comprises a 5′ UTR that comprises the sequence of SEQ ID NO: 2. In some embodiments, the hPIV3 RNA comprises a 3′ UTR that comprises the sequence of SEQ ID NO: 4.

In some embodiments, the hPIV3 RNA comprises a sequence that has at least 90%, at least 91%, at least 92%, at least 93%, at least 94%, at least 95%, at least 96%, at least 97%, at least 98%, at least 99%, or 100% identity to the sequence of SEQ ID NO: 17. In some embodiments, the hPIV3 RNA comprises the sequence of SEQ ID NO: 17.

In some embodiments, the hRSV RNA, the hMPV RNA, and/or the hPIV3 RNA further comprises a 7mG(5′)ppp(5′)NlmpNp cap.

In some embodiments, the hRSV RNA, the hMPV RNA, and/or the hPIV3 RNA further comprises a poly(A) tail, optionally having a length of 50 to 150 nucleotides.

In some embodiments, the hRSV RNA, the hMPV RNA, and/or the hPIV3 RNA comprises a chemical modification. In some embodiments, the chemical modification is 1-methylpseudouridine.

In some embodiments, a composition comprises 25 µg - 200 µg of the hRSV RNA, the hMPV RNA, and/or the hPIV3 RNA.

In some embodiments, a composition further comprises a mixture of lipids that comprises a PEG-modified lipid, a non-cationic lipid, a sterol, an ionizable cationic lipid, or any combination thereof.

In some embodiments, a mixture of lipids comprises 0.5-15% PEG-modified lipid; 5-25% non-cationic lipid; 25-55% sterol; and 20-60% ionizable cationic lipid.

In some embodiments, the PEG-modified lipid is 1,2 dimyristoyl-sn-glycerol, methoxypolyethyleneglycol (PEG2000 DMG), the non-cationic lipid is 1,2 distearoyl-sn-glycero-3-phosphocholine (DSPC), the sterol is cholesterol; and the ionizable cationic lipid has the structure of Compound 1:

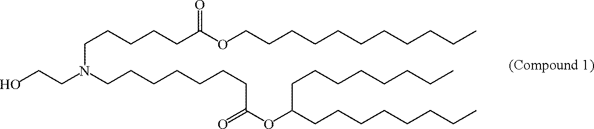 [SMILES: CCCCCCCCCCCOC(=O)CCCCCN(CCO)CCCCCCCC(=O)OC(CCCCCCCC)CCCCCCCC]

In some embodiments, a mixture of lipids forms lipid nanoparticles.

In some embodiments, the hRSV RNA, the hMPV RNA, and the hPIV3 RNA are formulated in the lipid nanoparticles.

Some aspects of the present disclosure provide a method comprising administering to a subject the composition of any one of the preceding claims in an amount effective to induce a neutralizing antibody response against hRSV, hMPV, and/or hPIV3 in the subject.

In some embodiments, the subject is immunocompromised. In some embodiments, the subject has a pulmonary disease.

In some embodiments, the subject is 5 years of age or younger. In other embodiments, the subject is 65 years of age or older.

In some embodiments, a method comprises administering to the subject at least two doses of the composition.

The entire contents of International Application No. PCT/US2016/058327 (Publication No. WO2017/07062) and International Application No. PCT/US2017/065408 (Publication No. WO2018/107088) are incorporated herein by reference.

### BRIEF DESCRIPTION OF THE DRAWINGS

FIG. 1 shows schematic representations of the RSV F glycoprotein variant (encoded by mRNA-1345) and wild-type RSV F glycoprotein.

FIGS. 2A-2B show in vitro screening of mRNAs having different features in HEK293T cells at two time points. AM14, a monoclonal antibody specific for the prefusion form of RSV F glycoprotein, was used for the flow cytometric analysis. In FIG. 2A, the top leads are shown at 24 hours and 48 hours post-transfection. Feature 1 refers to an optimized 5′ UTR and Feature 2 refers to 6 amino acid point mutations within the F1 region of wild-type RSV F glycoprotein (FIG. 1). FIG. 2B depicts expression levels after three different doses of the candidate constructs. “dCT” represents an mRNA encoding an RSV F glycoprotein having a truncated cytoplasmic tail.

FIGS. 3A-3B show in vivo screening of mRNAs having different features in mice. Following two doses of the mRNAs, post-fusion form RSV F protein levels (FIG. 3A) and the RSV-A neutralization titer (FIG. 3B) were measured.

FIGS. 4A-4B show the in vitro expression levels of the prefusion form RSV F glycoprotein using a codon-optimized mRNA encoding an RSV F glycoprotein having a truncated cytoplasmic tail. FIG. 4A shows the results at three different time points. FIG. 4B compares the combination of both features (codon optimization and cytoplasmic tail truncation) to mRNAs having each feature individually. AM14 and D25 are two prefusion RSV F glycoprotein-specific antibodies. Motavizumab detects both pre- and post-fusion RSV F glycoprotein.

FIGS. 5A-5B illustrate the in vitro expression levels of the prefusion form of RSV F glycoprotein using a codon-optimized mRNA encoding an RSV F glycoprotein with a truncated cytoplasmic tail in HEK293T cells (FIG. 5A) and THP-1 cells (FIG. 5B).

FIGS. 6A-6B show in vivo data for a codon-optimized mRNA encoding a prefusion form of RSV F glycoprotein with a truncated cytoplasmic tail. The post-fusion form of RSV F glycoprotein IgG titer levels resulting from administration of the mRNA are depicted after the first dose (FIG. 6A) and after the second dose (FIG. 6B).

FIG. 7 shows two graphs depicting the frequency of prefusion form RSV F glycoprotein-positive CD14+ monocytes following 24 hours (left) and 48 hours (right) incubation with the mRNA indicated (control (an mRNA encoding an alternative RSV F glycoprotein), RSV F variant, or no mRNA).

FIG. 8 is as graph depicting the frequency of prefusion form of RSV F glycoprotein-positive CD14+ monocytes following 24 hours (left) and 48 hours (right) incubation with the mRNA and concentration indicated (control (an mRNA encoding an alternative RSV F glycoprotein), a codon-optimized RSV F glycoprotein mRNA, an mRNA encoding an RSV F glycoprotein having a truncated cytoplasmic tail, an mRNA encoding the combination RSV F glycoprotein (codon-optimized with the cytoplasmic tail truncation), or no mRNA).

FIG. 9 is a graph showing the mean intensity per cell (averaged) following microscopy experiments. HeLa cells were incubated for 24 or 48 hours with 200 ng of the mRNAs indicated (control (an alternative mRNA encoding a RSV F glycoprotein), a codon-optimized mRNA encoding a RSV F glycoprotein, an mRNA encoding a RSV F glycoprotein having a truncated cytoplasmic tail, an mRNA encoding the combination RSV F glycoprotein (codon-optimized with the cytoplasmic tail truncation), or no mRNA) and the mean intensity was measured.

FIGS. 10A-10B show RSV antibody titers after vaccination on Day 56. FIG. 10A shows the RSV neutralizing titer and FIG. 10B shows the RSV prefusion F protein IgG titer. The groups marked with an asterisk (*) are those that only received one dose of the composition indicated. “Lot100” refers to the 1:100 formalin-inactivated RSV group (FI-RSV).

FIGS. 11A-11B show lung viral load (FIG. 11A) and nose viral load (FIG. 11B) after the RSV challenge (see Example 4). The groups marked with an asterisk (*) are those that only received one dose of the composition indicated. “Lot100” refers to the 1:100 formalin-inactivated RSV group (FI-RSV).

### DETAILED DESCRIPTION

The present disclosure provides immunizing compositions (e.g., RNA vaccines) that elicit potent neutralizing antibodies against respiratory virus antigens. The term “respiratory virus antigens” herein encompasses hRSV antigens (e.g., hRSV F glycoproteins), hMPV antigens (e.g., hMPV F glycoproteins), hPIV3 antigens (e.g., hPIV3 F glycoproteins), and any combination thereof (e.g., hRSV and hMPV, hRSV and hPIV3, hMPV and hPIV3, or hRSV, hMPV, and hPIV3) encoded by the RNA of the present disclosure. It should be understood that the terms “RNA” and “RNA construct” may be used interchangeably herein.

In some embodiments, an immunizing composition includes RNA (e.g., messenger RNA (mRNA)) encoding a prefusion form of hRSV F glycoprotein. In other embodiments, an immunizing composition further comprises RNA (e.g., mRNA) encoding a human metapneumovirus (hMPV) F glycoprotein. In still other embodiments, an immunizing composition further comprises RNA (e.g., mRNA) encoding a human parainfluenza virus 3 (hPIV3) F glycoprotein. In yet other embodiments, an immunizing composition includes an RNA (e.g., mRNA) encoding a prefusion form of hRSV F glycoprotein, RNA (e.g., mRNA) encoding an hMPV F glycoprotein, and RNA (e.g., mRNA) encoding an hPIV3 F glycoprotein. The prefusion form of hRSV F glycoprotein, the hMPV F glycoprotein, and the hPIV3 F glycoprotein, in some embodiments, are encoded by the same (a single) RNA, while in other embodiments, they are encoded independently by multiple RNAs (one encoding prefusion hRSV F, one encoding hMPV F, and one encoding hPIV3 F). In some embodiments, one RNA (e.g., having a 5′ UTR, ORF, 3′ UTR, and poly(A) tail) encodes the prefusion hRSV F glycoprotein and another RNA encodes both the hMPV F glycoprotein and the hPIV3 F glycoprotein.

The envelope of hRSV contains three surface glycoproteins: F, G, and SH. The G and F proteins are protective antigens and targets of neutralizing antibodies. The F protein, however, is more conserved across hRSV strains and types (A and B). hRSV F protein is a type I fusion glycoprotein that is well conserved between clinical isolates, including between the hRSV-A and hRSV-B antigenic subgroups. The F protein transitions between prefusion and more stable postfusion states, thereby facilitating entry into target cells. hRSV F glycoprotein is initially synthesized as an F0 precursor protein. hRSV F0 folds into a trimer, which is activated by furin cleavage into the mature prefusion protein comprising F1 and F2 subunits (Bolt, et al., Virus Res., 68:25, 2000). Although targets for neutralizing monoclonal antibodies exist on the postfusion conformation of F protein, the neutralizing Ab response primarily targets the F protein prefusion conformation in people naturally infected with hRSV (Magro M et al., Proc Natl Acad Sci USA 2012; 109(8): 3089-94; Ngwuta JO et al., Sci Transl Med 2015; 7(309): 309ra162). Consistent with this, hRSV F protein stabilized in the prefusion conformation produces a greater neutralizing immune response in animal models than that observed with hRSV F protein stabilized in the post fusion conformation (McLellan et al., Science, 342: 592-598, 2013). Thus, stabilized prefusion hRSV F proteins are good candidates for inclusion in an hRSV vaccine.

As used herein, stabilized prefusion RSV F proteins, which exist in a labile, high-energy state, are those that comprise mutations (e.g., stabilizing mutations) to prevent the transition of the protein into its post-fusion conformation. For example, in some embodiments, the stabilized prefusion RSV F protein comprises proline residue (e.g., an S215P substitution) and/or isoleucine (e.g., N67I substitution) substitutions. As an example, the DS-Cav1 variant, a stabilized prefusion RSV F protein, contains an additional disulfide bond (S155C/S290C) as well as two cavity-filling mutations (S190F/V207L). Another stabilized prefusion RSV F protein is PR-DM, which comprises one proline substitution (S215P) and one mutation in the F₂ subunit (N67I).

The hRSV RNA vaccines described herein are superior to current vaccines in several ways. For example, the lipid nanoparticle (LNP) delivery system used herein increases the efficacy of RNA vaccines in comparison to other formulations, including a protamine-based approach described in the literature. The use of this LNP delivery system enables the effective delivery of chemically-modified RNA vaccines or unmodified RNA vaccines, without requiring additional adjuvant to produce a therapeutic result (e.g., production neutralizing antibody titer). In some embodiments, the hRSV RNA vaccines disclosed herein are superior to conventional vaccines by a factor of at least 10 fold, 20, fold, 40, fold, 50 fold, 100 fold, 500 fold, or 1,000 fold when administered intramuscularly (IM) or intradermally (ID). These results can be achieved even when significantly lower doses of the RNA (e.g., mRNA) are administered in comparison with RNA doses used in other classes of lipid based formulations.

Further, unlike self-replicating RNA vaccines, which rely on viral replication pathways to deliver enough RNA to a cell to produce an immunogenic response, the compositions of the present disclosure do not require viral replication to produce enough protein to result in a strong immune response. Thus, the compositions of the present disclosure do not include self-replicating RNA and do not include components necessary for viral replication.

The RNAs provided herein are not limited by a particular strain of virus (e.g., hRSV, hMPV, and/or hPIV3). The strain of virus on which the mRNAs are based may be any strain of virus.

It should be understood that the immunizing compositions (e.g., RNA vaccines) of the present disclosure are not naturally-occurring. That is, the RNA polynucleotides encoding respiratory virus antigens, as provided herein, do not occur in nature. It should also be understood that the RNA polynucleotides described herein are isolated from viral proteins and viral lipids as they exist in nature. Thus, as provided herein, an immunizing composition comprising an RNA formulated in a lipid nanoparticle, for example, excludes viruses (i.e., the compositions are not, nor do they contain, viruses).

### Antigens

Antigens are proteins capable of inducing an immune response (e.g., causing an immune system to produce antibodies against the antigens). Herein, use of the term “antigen” encompasses immunogenic proteins and immunogenic fragments (an immunogenic fragment that induces (or is capable of inducing) an immune response to a (at least one) respiratory virus, e.g., hRSV, or hRSV, hMPV, and hPIV3), unless otherwise stated. It should be understood that the term “protein’ encompasses peptides and the term “antigen” encompasses antigenic fragments.

Exemplary sequences of the respiratory virus antigens and the RNA encoding the respiratory virus antigens of the compositions of the present disclosure are provided in Table 1.

In some embodiments, a composition comprises an RNA that encodes a prefusion form of an hRSV F glycoprotein that comprises the sequence of SEQ ID NO: 8. In some embodiments, a composition comprises an RNA that encodes a prefusion form of an hMPV F glycoprotein that comprises the sequence of SEQ ID NO: 11. In some embodiments, a composition comprises an RNA that encodes a prefusion form of an hPIV3 F glycoprotein that comprises the sequence of SEQ ID NO: 14.

It should be understood that any one of the antigens encoded by the RNA described herein may or may not comprise a signal sequence.

### Nucleic Acids

The compositions of the present disclosure comprise a (at least one) RNA having an open reading frame (ORF) encoding a respiratory virus antigen. In some embodiments, the RNA is a messenger RNA (mRNA). In some embodiments, the RNA (e.g., mRNA) further comprises a 5′ UTR, 3′ UTR, a poly(A) tail and/or a 5′ cap analog.

It should also be understood that the hMPV/hPIV3 mRNA vaccine of the present disclosure may include any 5′ untranslated region (UTR) and/or any 3′ UTR. Exemplary UTR sequences are provided in the Sequence Listing (e.g., SEQ ID NOs: 2-5); however, other UTR sequences may be used or exchanged for any of the UTR sequences described herein. UTRs may also be omitted from the RNA polynucleotides provided herein.

Nucleic acids comprise a polymer of nucleotides (nucleotide monomers). Thus, nucleic acids are also referred to as polynucleotides. Nucleic acids may be or may include, for example, deoxyribonucleic acids (DNAs), ribonucleic acids (RNAs), threose nucleic acids (TNAs), glycol nucleic acids (GNAs), peptide nucleic acids (PNAs), locked nucleic acids (LNAs, including LNA having a β-D-ribo configuration, α-LNA having an α-L-ribo configuration (a diastereomer of LNA), 2′-amino-LNA having a 2′-amino functionalization, and 2′-amino- α-LNA having a 2′-amino functionalization), ethylene nucleic acids (ENA), cyclohexenyl nucleic acids (CeNA) and/or chimeras and/or combinations thereof.

Messenger RNA (mRNA) is any RNA that encodes a (at least one) protein (a naturally-occurring, non-naturally-occurring, or modified polymer of amino acids) and can be translated to produce the encoded protein in vitro, in vivo, in situ, or ex vivo. The skilled artisan will appreciate that, except where otherwise noted, nucleic acid sequences set forth in the instant application may recite “T”s in a representative DNA sequence but where the sequence represents RNA (e.g., mRNA), the “T”s would be substituted for “U”s. Thus, any of the DNAs disclosed and identified by a particular sequence identification number herein also disclose the corresponding RNA (e.g., mRNA) sequence complementary to the DNA, where each “T” of the DNA sequence is substituted with “U.”

An open reading frame (ORF) is a continuous stretch of DNA or RNA beginning with a start codon (e.g., methionine (ATG or AUG)) and ending with a stop codon (e.g., TAA, TAG or TGA, or UAA, UAG or UGA). An ORF typically encodes a protein. It will be understood that the sequences disclosed herein may further comprise additional elements, e.g., 5′ and 3′ UTRs, but that those elements, unlike the ORF, need not necessarily be present in an RNA polynucleotide of the present disclosure.

### Variants

In some embodiments, the compositions of the present disclosure include RNA that encodes a respiratory virus antigen variant. Antigen variants or other polypeptide variants refers to molecules that differ in their amino acid sequence from a wild-type, native, or reference sequence. The antigen/polypeptide variants may possess substitutions, deletions, and/or insertions at certain positions within the amino acid sequence, as compared to a native or reference sequence. Ordinarily, variants possess at least 50% identity to a wild-type, native or reference sequence. In some embodiments, variants share at least 80%, or at least 90% identity with a wild-type, native, or reference sequence.

Variant antigens/polypeptides encoded by nucleic acids of the disclosure may contain amino acid changes that confer any of a number of desirable properties, e.g., that enhance their immunogenicity, enhance their expression, and/or improve their stability or PK/PD properties in a subject. Variant antigens/polypeptides can be made using routine mutagenesis techniques and assayed as appropriate to determine whether they possess the desired property. Assays to determine expression levels and immunogenicity are well known in the art and exemplary such assays are set forth in the Examples section. Similarly, PK/PD properties of a protein variant can be measured using art recognized techniques, e.g., by determining expression of antigens in a vaccinated subject over time and/or by looking at the durability of the induced immune response. The stability of protein(s) encoded by a variant nucleic acid may be measured by assaying thermal stability or stability upon urea denaturation or may be measured using in silico prediction. Methods for such experiments and in silico determinations are known in the art.

In some embodiments, a composition comprises an RNA or an RNA ORF that comprises a nucleotide sequence of any one of the sequences provided herein (see, e.g., Sequence Listing and Table 1), or comprises a nucleotide sequence at least 80%, at least 85%, at least 90%, at least 95%, at least 96%, at least 97%, at least 98%, or at least 99% identical to a nucleotide sequence of any one of the sequences provided herein.

The term “identity” refers to a relationship between the sequences of two or more polypeptides (e.g. antigens) or polynucleotides (nucleic acids), as determined by comparing the sequences. Identity also refers to the degree of sequence relatedness between or among sequences as determined by the number of matches between strings of two or more amino acid residues or nucleic acid residues. Identity measures the percent of identical matches between the smaller of two or more sequences with gap alignments (if any) addressed by a particular mathematical model or computer program (e.g., “algorithms”). Identity of related antigens or nucleic acids can be readily calculated by known methods. “Percent (%) identity” as it applies to polypeptide or polynucleotide sequences is defined as the percentage of residues (amino acid residues or nucleic acid residues) in the candidate amino acid or nucleic acid sequence that are identical with the residues in the amino acid sequence or nucleic acid sequence of a second sequence after aligning the sequences and introducing gaps, if necessary, to achieve the maximum percent identity. Methods and computer programs for the alignment are well known in the art. It is understood that identity depends on a calculation of percent identity but may differ in value due to gaps and penalties introduced in the calculation. Generally, variants of a particular polynucleotide or polypeptide (e.g., antigen) have at least 40%, 45%, 50%, 55%, 60%, 65%, 70%, 75%, 80%, 85%, 90%, 91%, 92%, 93%, 94%, 95%, 96%, 97%, 98%, 99% but less than 100% sequence identity to that particular reference polynucleotide or polypeptide as determined by sequence alignment programs and parameters described herein and known to those skilled in the art. Such tools for alignment include those of the BLAST suite (Stephen F. Altschul, et al (1997), “Gapped BLAST and PSI-BLAST: a new generation of protein database search programs”, Nucleic Acids Res. 25:3389-3402). Another popular local alignment technique is based on the Smith-Waterman algorithm (Smith, T.F. & Waterman, M.S. (1981) “Identification of common molecular subsequences.” J. Mol. Biol. 147:195-197). A general global alignment technique based on dynamic programming is the Needleman-Wunsch algorithm (Needleman, S.B. & Wunsch, C.D. (1970) “A general method applicable to the search for similarities in the amino acid sequences of two proteins.” J. Mol. Biol. 48:443-453). More recently a Fast Optimal Global Sequence Alignment Algorithm (FOGSAA) has been developed that purportedly produces global alignment of nucleotide and protein sequences faster than other optimal global alignment methods, including the Needleman-Wunsch algorithm.

As such, polynucleotides encoding peptides or polypeptides containing substitutions, insertions and/or additions, deletions and covalent modifications with respect to reference sequences, in particular the polypeptide (e.g., antigen) sequences disclosed herein, are included within the scope of this disclosure. For example, sequence tags or amino acids, such as one or more lysines, can be added to peptide sequences (e.g., at the N-terminal or C-terminal ends). Sequence tags can be used for peptide detection, purification or localization. Lysines can be used to increase peptide solubility or to allow for biotinylation. Alternatively, amino acid residues located at the carboxy and amino terminal regions of the amino acid sequence of a peptide or protein may optionally be deleted providing for truncated sequences. Certain amino acids (e.g., C-terminal or N-terminal residues) may alternatively be deleted depending on the use of the sequence, as for example, expression of the sequence as part of a larger sequence which is soluble, or linked to a solid support. In some embodiments, sequences for (or encoding) signal sequences, termination sequences, transmembrane domains, linkers, multimerization domains (such as, e.g., foldon regions) and the like may be substituted with alternative sequences that achieve the same or a similar function. In some embodiments, cavities in the core of proteins can be filled to improve stability, e.g., by introducing larger amino acids. In other embodiments, buried hydrogen bond networks may be replaced with hydrophobic resides to improve stability. In yet other embodiments, glycosylation sites may be removed and replaced with appropriate residues. Such sequences are readily identifiable to one of skill in the art. It should also be understood that some of the sequences provided herein contain sequence tags or terminal peptide sequences (e.g., at the N-terminal or C-terminal ends) that may be deleted, for example, prior to use in the preparation of an RNA (e.g., mRNA) vaccine.

As recognized by those skilled in the art, protein fragments, functional protein domains, and homologous proteins are also considered to be within the scope of respiratory virus antigens of interest. For example, provided herein is any protein fragment (meaning a polypeptide sequence at least one amino acid residue shorter than a reference antigen sequence but otherwise identical) of a reference protein, provided that the fragment is immunogenic and confers a protective immune response to the respiratory virus. In addition to variants that are identical to the reference protein but are truncated, in some embodiments, an antigen includes 2, 3, 4, 5, 6, 7, 8, 9, 10, or more mutations, as shown in any of the sequences provided or referenced herein. Antigens/antigenic polypeptides can range in length from about 4, 6, or 8 amino acids to full length proteins.

### hRSV Antigen Variants

In some embodiments, a composition comprises an RNA encoding a stabilized prefusion form of a hRSV F glycoprotein variant that lacks a cytoplasmic tail. In some embodiments, the cytoplasmic tail comprises the C-terminal 20-30, 20-25, 15-30, 15-25, 15-20, 10-30, 10-25, 10-20, 10-15, 5-30, 5-25, 5-20, or 5-15 amino acids of the of the hRSV F glycoprotein variant. In some embodiments, the cytoplasmic tail comprises the C-terminal 25 amino acids (e.g., CKARSTPVTLSKDQLSGINNIAFSN (SEQ ID NO: 25)) of the hRSV F glycoprotein. In some embodiments, the cytoplasmic tail comprises the C-terminal 20 amino acids (e.g., TPVTLSKDQLSGINNIAFSN (SEQ ID NO: 26)) of the hRSV F glycoprotein. In some embodiments, the cytoplasmic tail comprises the C-terminal 15 amino acids (e.g., SKDQLSGINNIAFSN (SEQ ID NO: 27)) of the hRSV F glycoprotein. In some embodiments, the cytoplasmic tail comprises the C-terminal 10 amino acids (e.g., SGINNIAFSN (SEQ ID NO: 28)) of the hRSV F glycoprotein.

In some embodiments, a composition comprises an RNA encoding a stabilized prefusion form of a hRSV F glycoprotein variant that lacks a cytoplasmic tail, wherein the RSV F glycoprotein variant has at least 80%, at least 85%, at least 90%, at least 95% identity to a wild-type hRSV F glycoprotein (e.g., a wild-type hRSV F glycoprotein comprising the sequence of SEQ ID NO: 1) or a wild-type hRSV F glycoprotein that lacks a cytoplasmic tail. In some embodiments, a composition comprises an RNA encoding a stabilized prefusion form of a hRSV F glycoprotein variant that lacks a cytoplasmic tail, wherein the RSV F glycoprotein variant has at least 80%, at least 85%, at least 90%, at least 95% identity to the sequence of SEQ ID NO: 8. In some embodiments, a composition comprises an RNA encoding a stabilized prefusion form of a hRSV F glycoprotein variant that comprises the sequence of SEQ ID NO: 8.

In some embodiments, a composition comprises an RNA encoding a stabilized prefusion form of a hRSV F glycoprotein variant that that lacks a cytoplasmic tail, wherein the RNA comprises an ORF sequence that has at least 80%, at least 85%, at least 90%, at least 95% identity to the sequence of SEQ ID NO: 7. In some embodiments, a composition comprises an RNA encoding a stabilized prefusion form of a hRSV F glycoprotein variant that lacks a cytoplasmic tail, wherein the RNA comprises a sequence that has at least 80%, at least 85%, at least 90%, at least 95% identity to the sequence of SEQ ID NO: 15.

In some embodiments, an hRSV F glycoprotein variant that lacks a cytoplasmic tail further comprises a modification, relative to the wild-type hRSV F glycoprotein (e.g., SEQ ID NO: 1), selected from the group consisting of: a P102X substitution, a substitution of amino acids 104-144 with a linker molecule, an A149X substitution, an S155X substitution, an S190X substitution, a V207X substitution, an S290X substitution, a L373X substitution, an I379X substitution, an M447X substitution, and a Y458X substitution, wherein X is any amino acid (e.g., A, R, N, D, C, E, Q, G, H, I, L, K, M, F, P, S, T, W, Y, or V).

In some embodiments, an hRSV F glycoprotein variant that lacks a cytoplasmic tail further comprises a modification, relative to the wild-type hRSV F glycoprotein, selected from the group consisting of: a P102A substitution, a substitution of amino acids 104-144 with a linker molecule, an A149C substitution, an S155C substitution, an S190F substitution, a V207L substitution, an S290C substitution, a L373R substitution, an I379V substitution, an M447V substitution, and a Y458C substitution. In some embodiments, an hRSV F glycoprotein variant that lacks a cytoplasmic tail further comprises a P102A substitution. In some embodiments, an hRSV F glycoprotein variant that lacks a cytoplasmic tail further comprises a substitution of amino acids 104-144 with a linker molecule. In some embodiments, an hRSV F glycoprotein variant that lacks a cytoplasmic tail further comprises an A149C substitution. In some embodiments, an hRSV F glycoprotein variant that lacks a cytoplasmic tail further comprises an S155C substitution. In some embodiments, an hRSV F glycoprotein variant that lacks a cytoplasmic tail further comprises an S190F substitution. In some embodiments, an hRSV F glycoprotein variant that lacks a cytoplasmic tail further comprises a V207L substitution. In some embodiments, an hRSV F glycoprotein variant that lacks a cytoplasmic tail further comprises an S290C substitution. In some embodiments, an hRSV F glycoprotein variant that lacks a cytoplasmic tail further comprises an L373R substitution. In some embodiments, an hRSV F glycoprotein variant that lacks a cytoplasmic tail further comprises an I379V substitution. In some embodiments, an hRSV F glycoprotein variant that lacks a cytoplasmic tail further comprises an M447V substitution. In some embodiments, an hRSV F glycoprotein variant that lacks a cytoplasmic tail further comprises a Y458C substitution.

In some embodiments, an hRSV F glycoprotein variant that lacks a cytoplasmic tail further comprises the following modifications, relative to the wild-type hRSV F glycoprotein: a P102A substitution, a substitution of amino acids 104-144 with a linker molecule, an A149C substitution, an S155C substitution, an S190F substitution, a V207L substitution, an S290C substitution, a L373R substitution, an I379V substitution, an M447V substitution, and a Y458C substitution.

### hMPV Antigen Variants

In some embodiments, a composition comprises an RNA encoding an hMPV F glycoprotein variant that has at least 80%, at least 85%, at least 90%, at least 95% identity to a wild-type hMPV F glycoprotein. In some embodiments, a composition comprises an RNA encoding an hMPV F glycoprotein variant has at least 80%, at least 85%, at least 90%, at least 95% identity to the sequence of SEQ ID NO: 11. In some embodiments, a composition comprises an RNA encoding an hMPV F glycoprotein variant that comprises the sequence of SEQ ID NO: 11.

In some embodiments, a composition comprises an RNA encoding an hMPV F glycoprotein variant, wherein the RNA comprises an ORF sequence that has at least 80%, at least 85%, at least 90%, at least 95% identity to the sequence of SEQ ID NO: 10. In some embodiments, a composition comprises an RNA encoding an hMPV F glycoprotein variant, wherein the RNA comprises a sequence that has at least 80%, at least 85%, at least 90%, at least 95% identity to the sequence of SEQ ID NO: 16.

### hPIV3 Antigen Variants

In some embodiments, a composition comprises an RNA encoding an hPIV3 F glycoprotein variant that has at least 80%, at least 85%, at least 90%, at least 95% identity to a wild-type hPIV3 F glycoprotein. In some embodiments, a composition comprises an RNA encoding an hPIV3 F glycoprotein variant has at least 80%, at least 85%, at least 90%, at least 95% identity to the sequence of SEQ ID NO: 14. In some embodiments, a composition comprises an RNA encoding an hPIV3 F glycoprotein variant that comprises the sequence of SEQ ID NO: 14.

In some embodiments, a composition comprises an RNA encoding an hPIV3 F glycoprotein variant, wherein the RNA comprises an ORF sequence that has at least 80%, at least 85%, at least 90%, at least 95% identity to the sequence of SEQ ID NO: 13. In some embodiments, a composition comprises an RNA encoding an hPIV3 F glycoprotein variant, wherein the RNA comprises a sequence that has at least 80%, at least 85%, at least 90%, at least 95% identity to the sequence of SEQ ID NO: 17.

### Stabilizing Elements

Naturally-occurring eukaryotic mRNA molecules can contain stabilizing elements, including, but not limited to untranslated regions (UTR) at their 5′-end (5′ UTR) and/or at their 3′-end (3′ UTR), in addition to other structural features, such as a 5′-cap structure or a 3′-poly(A) tail. Both the 5′ UTR and the 3′ UTR are typically transcribed from the genomic DNA and are elements of the premature mRNA. Characteristic structural features of mature mRNA, such as the 5′-cap and the 3′-poly(A) tail are usually added to the transcribed (premature) mRNA during mRNA processing.

In some embodiments, a composition includes an RNA polynucleotide having an open reading frame encoding at least one antigenic polypeptide having at least one modification, at least one 5′ terminal cap, and is formulated within a lipid nanoparticle. 5′-capping of polynucleotides may be completed concomitantly during the in vitro-transcription reaction using the following chemical RNA cap analogs to generate the 5′-guanosine cap structure according to manufacturer protocols: 3′-O-Me-m7G(5′)ppp(5′) G [the ARCA cap];G(5′)ppp(5′)A; G(5′)ppp(5′)G; m7G(5′)ppp(5′)A; m7G(5′)ppp(5′)G (New England BioLabs, Ipswich, MA). 5′-capping of modified RNA may be completed post-transcriptionally using a Vaccinia Virus Capping Enzyme to generate the “Cap 0” structure: m7G(5′)ppp(5′)G (New England BioLabs, Ipswich, MA). Cap 1 structure may be generated using both Vaccinia Virus Capping Enzyme and a 2′-O methyl-transferase to generate: m7G(5′)ppp(5′)G-2′-O-methyl. Cap 2 structure may be generated from the Cap 1 structure followed by the 2′-O-methylation of the 5′-antepenultimate nucleotide using a 2′-O methyl-transferase. Cap 3 structure may be generated from the Cap 2 structure followed by the 2′-O-methylation of the 5′-preantepenultimate nucleotide using a 2′-O methyl-transferase. Enzymes may be derived from a recombinant source.

The 3′-poly(A) tail is typically a stretch of adenine nucleotides added to the 3′-end of the transcribed mRNA. It can, in some instances, comprise up to about 400 adenine nucleotides. In some embodiments, the length of the 3′-poly(A) tail may be an essential element with respect to the stability of the individual mRNA.

In some embodiments, a composition includes a stabilizing element. Stabilizing elements may include for instance a histone stem-loop. A stem-loop binding protein (SLBP), a 32 kDa protein has been identified. It is associated with the histone stem-loop at the 3′-end of the histone messages in both the nucleus and the cytoplasm. Its expression level is regulated by the cell cycle; it peaks during the S-phase, when histone mRNA levels are also elevated. The protein has been shown to be essential for efficient 3′-end processing of histone pre-mRNA by the U7 snRNP. SLBP continues to be associated with the stem-loop after processing, and then stimulates the translation of mature histone mRNAs into histone proteins in the cytoplasm. The RNA binding domain of SLBP is conserved through metazoa and protozoa; its binding to the histone stem-loop depends on the structure of the loop. The minimum binding site includes at least three nucleotides 5′ and two nucleotides 3′ relative to the stem-loop.

In some embodiments, an RNA (e.g., mRNA) includes a coding region, at least one histone stem-loop, and optionally, a poly(A) sequence or polyadenylation signal. The poly(A) sequence or polyadenylation signal generally should enhance the expression level of the encoded protein. The encoded protein, in some embodiments, is not a histone protein, a reporter protein (e.g. Luciferase, GFP, EGFP, β-Galactosidase, EGFP), or a marker or selection protein (e.g. alpha-Globin, Galactokinase and Xanthine:guanine phosphoribosyl transferase (GPT)).

In some embodiments, an RNA (e.g., mRNA) includes the combination of a poly(A) sequence or polyadenylation signal and at least one histone stem-loop, even though both represent alternative mechanisms in nature, acts synergistically to increase the protein expression beyond the level observed with either of the individual elements. The synergistic effect of the combination of poly(A) and at least one histone stem-loop does not depend on the order of the elements or the length of the poly(A) sequence.

In some embodiments, an RNA (e.g., mRNA) does not include a histone downstream element (HDE). “Histone downstream element” (HDE) includes a purine-rich polynucleotide stretch of approximately 15 to 20 nucleotides 3′ of naturally occurring stem-loops, representing the binding site for the U7 snRNA, which is involved in processing of histone pre-mRNA into mature histone mRNA. In some embodiments, the nucleic acid does not include an intron.

An RNA (e.g., mRNA) may or may not contain an enhancer and/or promoter sequence, which may be modified or unmodified or which may be activated or inactivated. In some embodiments, the histone stem-loop is generally derived from histone genes, and includes an intramolecular base pairing of two neighbored partially or entirely reverse complementary sequences separated by a spacer, consisting of a short sequence, which forms the loop of the structure. The unpaired loop region is typically unable to base pair with either of the stem loop elements. It occurs more often in RNA, as is a key component of many RNA secondary structures, but may be present in single-stranded DNA as well. Stability of the stem-loop structure generally depends on the length, number of mismatches or bulges, and base composition of the paired region. In some embodiments, wobble base pairing (non-Watson-Crick base pairing) may result. In some embodiments, the at least one histone stem-loop sequence comprises a length of 15 to 45 nucleotides.

In some embodiments, an RNA (e.g., mRNA) has one or more AU-rich sequences removed. These sequences, sometimes referred to as AURES are destabilizing sequences found in the 3′UTR. The AURES may be removed from the RNA vaccines. Alternatively the AURES may remain in the RNA vaccine.

### Signal Peptides

In some embodiments, a composition comprises an RNA (e.g., mRNA) having an ORF that encodes a signal peptide fused to the respiratory virus antigen. Signal peptides, comprising the N-terminal 15-60 amino acids of proteins, are typically needed for the translocation across the membrane on the secretory pathway and, thus, universally control the entry of most proteins both in eukaryotes and prokaryotes to the secretory pathway. In eukaryotes, the signal peptide of a nascent precursor protein (pre-protein) directs the ribosome to the rough endoplasmic reticulum (ER) membrane and initiates the transport of the growing peptide chain across it for processing. ER processing produces mature proteins, wherein the signal peptide is cleaved from precursor proteins, typically by a ER-resident signal peptidase of the host cell, or they remain uncleaved and function as a membrane anchor. A signal peptide may also facilitate the targeting of the protein to the cell membrane.

A signal peptide may have a length of 15-60 amino acids. For example, a signal peptide may have a length of 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 amino acids. In some embodiments, a signal peptide has a length of 20-60, 25-60, 30-60, 35-60, 40-60, 45- 60, 50-60, 55-60, 15-55, 20-55, 25-55, 30-55, 35-55, 40-55, 45-55, 50-55, 15-50, 20-50, 25-50, 30-50, 35-50, 40-50, 45-50, 15-45, 20-45, 25-45, 30-45, 35-45, 40-45, 15-40, 20-40, 25-40, 30-40, 35-40, 15-35, 20-35, 25-35, 30-35, 15-30, 20-30, 25-30, 15-25, 20-25, or 15-20 amino acids.

Signal peptides from heterologous genes (which regulate expression of genes other than respiratory virus antigens in nature) are known in the art and can be tested for desired properties and then incorporated into a nucleic acid of the disclosure. In some embodiments, the signal peptide may comprise one of the following sequences: MDSKGSSQKGSRLLLLLVVSNLLLPQGVVG (SEQ ID NO: 18), MDWTWILFLVAAATRVHS (SEQ ID NO: 19); METPAQLLFLLLLWLPDTTG (SEQ ID NO: 20); MLGSNSGQRVVFTILLLLVAPAYS (SEQ ID NO: 21); MKCLLYLAFLFIGVNCA (SEQ ID NO: 22); MWLVSLAIVTACAGA (SEQ ID NO: 23).

### Fusion Proteins

In some embodiments, a composition of the present disclosure includes an RNA (e.g., mRNA) encoding an antigenic fusion protein. Thus, the encoded antigen or antigens may include two or more proteins (e.g., protein and/or protein fragment) joined together. In some embodiments, the RNA encodes a hMPV F glycoprotein fused to a hPIV3 F glycoprotein. Alternatively, the protein to which a protein antigen is fused does not promote a strong immune response to itself, but rather to the respiratory virus antigen. Antigenic fusion proteins, in some embodiments, retain the functional property from each original protein.

### Scaffold Moieties

The RNA (e.g., mRNA) vaccines as provided herein, in some embodiments, encode fusion proteins that comprise respiratory virus antigens linked to scaffold moieties. In some embodiments, such scaffold moieties impart desired properties to an antigen encoded by a nucleic acid of the disclosure. For example scaffold proteins may improve the immunogenicity of an antigen, e.g., by altering the structure of the antigen, altering the uptake and processing of the antigen, and/or causing the antigen to bind to a binding partner.

In some embodiments, the scaffold moiety is protein that can self-assemble into protein nanoparticles that are highly symmetric, stable, and structurally organized, with diameters of 10-150 nm, a highly suitable size range for optimal interactions with various cells of the immune system. In some embodiments, viral proteins or virus-like particles can be used to form stable nanoparticle structures. Examples of such viral proteins are known in the art. For example, in some embodiments, the scaffold moiety is a hepatitis B surface antigen (HBsAg). HBsAg forms spherical particles with an average diameter of ~22 nm and which lacked nucleic acid and hence are non-infectious (Lopez-Sagaseta, J. et al. Computational and Structural Biotechnology Journal 14 (2016) 58-68). In some embodiments, the scaffold moiety is a hepatitis B core antigen (HBcAg) self-assembles into particles of 24-31 nm diameter, which resembled the viral cores obtained from HBV-infected human liver. HBcAg produced in self-assembles into two classes of differently sized nanoparticles of 300 Å and 360 Å diameter, corresponding to 180 or 240 protomers. In some embodiments, the respiratory virus antigen is fused to HBsAG or HBcAG to facilitate self-assembly of nanoparticles displaying the respiratory virus antigen.

In some embodiments, bacterial protein platforms may be used. Non-limiting examples of these self-assembling proteins include ferritin, lumazine and encapsulin.

Ferritin is a protein whose main function is intracellular iron storage. Ferritin is made of 24 subunits, each composed of a four-alpha-helix bundle, that self-assemble in a quaternary structure with octahedral symmetry (Cho K.J. et al. J Mol Biol. 2009;390:83-98). Several high-resolution structures of ferritin have been determined, confirming that Helicobacter pylori ferritin is made of 24 identical protomers, whereas in animals, there are ferritin light and heavy chains that can assemble alone or combine with different ratios into particles of 24 subunits (Granier T. et al. J Biol Inorg Chem. 2003;8:105-111; Lawson D.M. et al. Nature. 1991;349:541-544). Ferritin self-assembles into nanoparticles with robust thermal and chemical stability. Thus, the ferritin nanoparticle is well-suited to carry and expose antigens.

Lumazine synthase (LS) is also well-suited as a nanoparticle platform for antigen display. LS, which is responsible for the penultimate catalytic step in the biosynthesis of riboflavin, is an enzyme present in a broad variety of organisms, including archaea, bacteria, fungi, plants, and eubacteria (Weber S.E. Flavins and Flavoproteins. Methods and Protocols, Series: Methods in Molecular Biology. 2014). The LS monomer is 150 amino acids long, and consists of beta-sheets along with tandem alpha-helices flanking its sides. A number of different quaternary structures have been reported for LS, illustrating its morphological versatility: from homopentamers up to symmetrical assemblies of 12 pentamers forming capsids of 150 Å diameter. Even LS cages of more than 100 subunits have been described (Zhang X. et al. J Mol Biol. 2006;362:753-770).

Encapsulin, a novel protein cage nanoparticle isolated from thermophile Thermotoga maritima, may also be used as a platform to present antigens on the surface of self-assembling nanoparticles. Encapsulin is assembled from 60 copies of identical 31 kDa monomers having a thin and icosahedral T = 1 symmetric cage structure with interior and exterior diameters of 20 and 24 nm, respectively (Sutter M. et al. Nat Struct Mol Biol. 2008, 15: 939-947). Although the exact function of encapsulin in T. maritima is not clearly understood yet, its crystal structure has been recently solved and its function was postulated as a cellular compartment that encapsulates proteins such as DyP (Dye decolorizing peroxidase) and Flp (Ferritin like protein), which are involved in oxidative stress responses (Rahmanpour R. et al. FEBS J. 2013, 280: 2097-2104).

### Linkers and Cleavable Peptides

In some embodiments, the mRNAs of the disclosure encode more than one polypeptide, referred to herein as fusion proteins. In some embodiments, the mRNA further encodes a linker located between at least one or each domain of the fusion protein. The linker can be, for example, a cleavable linker or protease-sensitive linker. In some embodiments, the linker is selected from the group consisting of F2A linker, P2A linker, T2A linker, E2A linker, and combinations thereof. This family of self-cleaving peptide linkers, referred to as 2A peptides, has been described in the art (see for example, Kim, J.H. et al. (2011) PLoS ONE 6:e18556). In some embodiments, the linker is an F2A linker. In some embodiments, the linker is a GGGS linker. In some embodiments, the fusion protein contains three domains with intervening linkers, having the structure: domain-linker-domain-linker-domain.

Cleavable linkers known in the art may be used in connection with the disclosure. Exemplary such linkers include: F2A linkers, T2A linkers, P2A linkers, E2A linkers (See, e.g., WO2017127750). The skilled artisan will appreciate that other art-recognized linkers may be suitable for use in the RNAs disclosure (e.g., encoded by the nucleic acids of the disclosure). The skilled artisan will likewise appreciate that other polycistronic RNA (e.g., mRNA encoding more than one antigen/polypeptide separately within the same molecule) may be suitable for use as provided herein.

### Sequence Optimization

In some embodiments, an ORF encoding an antigen of the disclosure is codon optimized. Codon optimization methods are known in the art. For example, an ORF of any one or more of the sequences provided herein may be codon optimized. Codon optimization, in some embodiments, may be used to match codon frequencies in target and host organisms to ensure proper folding; bias GC content to increase mRNA stability or reduce secondary structures; minimize tandem repeat codons or base runs that may impair gene construction or expression; customize transcriptional and translational control regions; insert or remove protein trafficking sequences; remove/add post translation modification sites in encoded protein (e.g., glycosylation sites); add, remove or shuffle protein domains; insert or delete restriction sites; modify ribosome binding sites and mRNA degradation sites; adjust translational rates to allow the various domains of the protein to fold properly; or reduce or eliminate problem secondary structures within the polynucleotide. Codon optimization tools, algorithms and services are known in the art - non-limiting examples include services from GeneArt (Life Technologies), DNA2.0 (Menlo Park CA) and/or proprietary methods. In some embodiments, the open reading frame (ORF) sequence is optimized using optimization algorithms.

In some embodiments, a codon optimized sequence shares less than 95% sequence identity to a naturally-occurring or wild-type sequence ORF (e.g., a naturally-occurring or wild-type mRNA sequence encoding a respiratory virus antigen). In some embodiments, a codon optimized sequence shares less than 90% sequence identity to a naturally-occurring or wild-type sequence (e.g., a naturally-occurring or wild-type mRNA sequence encoding a respiratory virus antigen). In some embodiments, a codon optimized sequence shares less than 85% sequence identity to a naturally-occurring or wild-type sequence (e.g., a naturally-occurring or wild-type mRNA sequence encoding a respiratory virus antigen). In some embodiments, a codon optimized sequence shares less than 80% sequence identity to a naturally-occurring or wild-type sequence (e.g., a naturally-occurring or wild-type mRNA sequence encoding a respiratory virus antigen). In some embodiments, a codon optimized sequence shares less than 75% sequence identity to a naturally-occurring or wild-type sequence (e.g., a naturally-occurring or wild-type mRNA sequence encoding a respiratory virus antigen).

In some embodiments, a codon optimized sequence shares between 65% and 85% (e.g., between about 67% and about 85% or between about 67% and about 80%) sequence identity to a naturally-occurring or wild-type sequence (e.g., a naturally-occurring or wild-type mRNA sequence encoding a respiratory virus antigen). In some embodiments, a codon optimized sequence shares between 65% and 75% or about 80% sequence identity to a naturally-occurring or wild-type sequence (e.g., a naturally-occurring or wild-type mRNA sequence encoding a respiratory virus antigen).

In some embodiments, a codon-optimized sequence encodes an antigen that is as immunogenic as, or more immunogenic than (e.g., at least 10%, at least 20%, at least 30%, at least 40%, at least 50%, at least 100%, or at least 200% more), than a respiratory virus antigen encoded by a non-codon-optimized sequence.

When transfected into mammalian host cells, the modified mRNAs have a stability of between 12-18 hours, or greater than 18 hours, e.g., 24, 36, 48, 60, 72, or greater than 72 hours and are capable of being expressed by the mammalian host cells.

In some embodiments, a codon optimized RNA may be one in which the levels of G/C are enhanced. The G/C-content of nucleic acid molecules (e.g., mRNA) may influence the stability of the RNA. RNA having an increased amount of guanine (G) and/or cytosine (C) residues may be functionally more stable than RNA containing a large amount of adenine (A) and thymine (T) or uracil (U) nucleotides. As an example, WO02/098443 discloses a pharmaceutical composition containing an mRNA stabilized by sequence modifications in the translated region. Due to the degeneracy of the genetic code, the modifications work by substituting existing codons for those that promote greater RNA stability without changing the resulting amino acid. The approach is limited to coding regions of the RNA.

### Chemically Unmodified Nucleotides

In some embodiments, an RNA (e.g., mRNA) is not chemically modified and comprises the standard ribonucleotides consisting of adenosine, guanosine, cytosine and uridine. In some embodiments, nucleotides and nucleosides of the present disclosure comprise standard nucleoside residues such as those present in transcribed RNA (e.g. A, G, C, or U). In some embodiments, nucleotides and nucleosides of the present disclosure comprise standard deoxyribonucleosides such as those present in DNA (e.g. dA, dG, dC, or dT).

### Chemical Modifications

The compositions of the present disclosure comprise, in some embodiments, an RNA having an open reading frame encoding a respiratory virus antigen, wherein the nucleic acid comprises nucleotides and/or nucleosides that can be standard (unmodified) or modified as is known in the art. In some embodiments, nucleotides and nucleosides of the present disclosure comprise modified nucleotides or nucleosides. Such modified nucleotides and nucleosides can be naturally-occurring modified nucleotides and nucleosides or non-naturally occurring modified nucleotides and nucleosides. Such modifications can include those at the sugar, backbone, or nucleobase portion of the nucleotide and/or nucleoside as are recognized in the art.

In some embodiments, a naturally-occurring modified nucleotide or nucleotide of the disclosure is one as is generally known or recognized in the art. Non-limiting examples of such naturally occurring modified nucleotides and nucleotides can be found, inter alia, in the widely recognized MODOMICS database.

In some embodiments, a non-naturally occurring modified nucleotide or nucleoside of the disclosure is one as is generally known or recognized in the art. Non-limiting examples of such non-naturally occurring modified nucleotides and nucleosides can be found, inter alia, in published U.S. Application Nos. PCT/US2012/058519; PCT/US2013/075177; PCT/US2014/058897; PCT/US2014/058891; PCT/US2014/070413; PCT/US2015/36773; PCT/US2015/36759; PCT/US2015/36771; or PCT/IB2017/051367 all of which are incorporated by reference herein.

Hence, nucleic acids of the disclosure (e.g., DNA nucleic acids and RNA nucleic acids, such as mRNA nucleic acids) can comprise standard nucleotides and nucleosides, naturally-occurring nucleotides and nucleosides, non-naturally-occurring nucleotides and nucleosides, or any combination thereof.

Nucleic acids of the disclosure (e.g., DNA nucleic acids and RNA nucleic acids, such as mRNA nucleic acids), in some embodiments, comprise various (more than one) different types of standard and/or modified nucleotides and nucleosides. In some embodiments, a particular region of a nucleic acid contains one, two or more (optionally different) types of standard and/or modified nucleotides and nucleosides.

In some embodiments, a modified RNA nucleic acid (e.g., a modified mRNA nucleic acid), introduced to a cell or organism, exhibits reduced degradation in the cell or organism, respectively, relative to an unmodified nucleic acid comprising standard nucleotides and nucleosides.

In some embodiments, a modified RNA nucleic acid (e.g., a modified mRNA nucleic acid), introduced into a cell or organism, may exhibit reduced immunogenicity in the cell or organism, respectively (e.g., a reduced innate response) relative to an unmodified nucleic acid comprising standard nucleotides and nucleosides.

Nucleic acids (e.g., RNA nucleic acids, such as mRNA nucleic acids), in some embodiments, comprise non-natural modified nucleotides that are introduced during synthesis or post-synthesis of the nucleic acids to achieve desired functions or properties. The modifications may be present on internucleotide linkages, purine or pyrimidine bases, or sugars. The modification may be introduced with chemical synthesis or with a polymerase enzyme at the terminal of a chain or anywhere else in the chain. Any of the regions of a nucleic acid may be chemically modified.

The present disclosure provides for modified nucleosides and nucleotides of a nucleic acid (e.g., RNA nucleic acids, such as mRNA nucleic acids). A “nucleoside” refers to a compound containing a sugar molecule (e.g., a pentose or ribose) or a derivative thereof in combination with an organic base (e.g., a purine or pyrimidine) or a derivative thereof (also referred to herein as “nucleobase”). A “nucleotide” refers to a nucleoside, including a phosphate group. Modified nucleotides may by synthesized by any useful method, such as, for example, chemically, enzymatically, or recombinantly, to include one or more modified or non-natural nucleosides. Nucleic acids can comprise a region or regions of linked nucleosides. Such regions may have variable backbone linkages. The linkages can be standard phosphodiester linkages, in which case the nucleic acids would comprise regions of nucleotides.

Modified nucleotide base pairing encompasses not only the standard adenosine-thymine, adenosine-uracil, or guanosine-cytosine base pairs, but also base pairs formed between nucleotides and/or modified nucleotides comprising non-standard or modified bases, wherein the arrangement of hydrogen bond donors and hydrogen bond acceptors permits hydrogen bonding between a non-standard base and a standard base or between two complementary non-standard base structures, such as, for example, in those nucleic acids having at least one chemical modification. One example of such non-standard base pairing is the base pairing between the modified nucleotide inosine and adenine, cytosine or uracil. Any combination of base/sugar or linker may be incorporated into nucleic acids of the present disclosure.

In some embodiments, modified nucleobases in nucleic acids (e.g., RNA nucleic acids, such as mRNA nucleic acids) comprise 1-methyl-pseudouridine (m1ψ), 1-ethyl-pseudouridine (e1ψ), 5-methoxy-uridine (mo5U), 5-methyl-cytidine (m5C), and/or pseudouridine (ψ). In some embodiments, modified nucleobases in nucleic acids (e.g., RNA nucleic acids, such as mRNA nucleic acids) comprise 5-methoxymethyl uridine, 5-methylthio uridine, 1-methoxymethyl pseudouridine, 5-methyl cytidine, and/or 5-methoxy cytidine. In some embodiments, the polyribonucleotide includes a combination of at least two (e.g., 2, 3, 4 or more) of any of the aforementioned modified nucleobases, including but not limited to chemical modifications.

In some embodiments, a mRNA of the disclosure comprises 1-methyl-pseudouridine (m1ψ) substitutions at one or more or all uridine positions of the nucleic acid.

In some embodiments, a mRNA of the disclosure comprises 1-methyl-pseudouridine (m1ψ) substitutions at one or more or all uridine positions of the nucleic acid and 5-methyl cytidine substitutions at one or more or all cytidine positions of the nucleic acid.

In some embodiments, a mRNA of the disclosure comprises pseudouridine (ψ) substitutions at one or more or all uridine positions of the nucleic acid.

In some embodiments, a mRNA of the disclosure comprises pseudouridine (ψ) substitutions at one or more or all uridine positions of the nucleic acid and 5-methyl cytidine substitutions at one or more or all cytidine positions of the nucleic acid..

In some embodiments, a mRNA of the disclosure comprises uridine at one or more or all uridine positions of the nucleic acid.

In some embodiments, mRNAs are uniformly modified (e.g., fully modified, modified throughout the entire sequence) for a particular modification. For example, a nucleic acid can be uniformly modified with 1-methyl-pseudouridine, meaning that all uridine residues in the mRNA sequence are replaced with 1-methyl-pseudouridine. Similarly, a nucleic acid can be uniformly modified for any type of nucleoside residue present in the sequence by replacement with a modified residue such as those set forth above.

The nucleic acids of the present disclosure may be partially or fully modified along the entire length of the molecule. For example, one or more or all or a given type of nucleotide (e.g., purine or pyrimidine, or any one or more or all of A, G, U, C) may be uniformly modified in a nucleic acid of the disclosure, or in a predetermined sequence region thereof (e.g., in the mRNA including or excluding the poly(A) tail). In some embodiments, all nucleotides X in a nucleic acid of the present disclosure (or in a sequence region thereof) are modified nucleotides, wherein X may be any one of nucleotides A, G, U, C, or any one of the combinations A+G, A+U, A+C, G+U, G+C, U+C, A+G+U, A+G+C, G+U+C or A+G+C.

The nucleic acid may contain from about 1% to about 100% modified nucleotides (either in relation to overall nucleotide content, or in relation to one or more types of nucleotide, i.e., any one or more of A, G, U or C) or any intervening percentage (e.g., from 1% to 20%, from 1% to 25%, from 1% to 50%, from 1% to 60%, from 1% to 70%, from 1% to 80%, from 1% to 90%, from 1% to 95%, from 10% to 20%, from 10% to 25%, from 10% to 50%, from 10% to 60%, from 10% to 70%, from 10% to 80%, from 10% to 90%, from 10% to 95%, from 10% to 100%, from 20% to 25%, from 20% to 50%, from 20% to 60%, from 20% to 70%, from 20% to 80%, from 20% to 90%, from 20% to 95%, from 20% to 100%, from 50% to 60%, from 50% to 70%, from 50% to 80%, from 50% to 90%, from 50% to 95%, from 50% to 100%, from 70% to 80%, from 70% to 90%, from 70% to 95%, from 70% to 100%, from 80% to 90%, from 80% to 95%, from 80% to 100%, from 90% to 95%, from 90% to 100%, and from 95% to 100%). It will be understood that any remaining percentage is accounted for by the presence of unmodified A, G, U, or C.

The mRNAs may contain at a minimum 1% and at maximum 100% modified nucleotides, or any intervening percentage, such as at least 5% modified nucleotides, at least 10% modified nucleotides, at least 25% modified nucleotides, at least 50% modified nucleotides, at least 80% modified nucleotides, or at least 90% modified nucleotides. For example, the nucleic acids may contain a modified pyrimidine such as a modified uracil or cytosine. In some embodiments, at least 5%, at least 10%, at least 25%, at least 50%, at least 80%, at least 90% or 100% of the uracil in the nucleic acid is replaced with a modified uracil (e.g., a 5-substituted uracil). The modified uracil can be replaced by a compound having a single unique structure, or can be replaced by a plurality of compounds having different structures (e.g., 2, 3, 4 or more unique structures). In some embodiments, at least 5%, at least 10%, at least 25%, at least 50%, at least 80%, at least 90% or 100% of the cytosine in the nucleic acid is replaced with a modified cytosine (e.g., a 5-substituted cytosine). The modified cytosine can be replaced by a compound having a single unique structure, or can be replaced by a plurality of compounds having different structures (e.g., 2, 3, 4 or more unique structures).

### Untranslated Regions (UTRs)

The mRNAs of the present disclosure may comprise one or more regions or parts which act or function as an untranslated region. Where mRNAs are designed to encode at least one antigen of interest, the nucleic may comprise one or more of these untranslated regions (UTRs). Wild-type untranslated regions of a nucleic acid are transcribed but not translated. In mRNA, the 5′ UTR starts at the transcription start site and continues to the start codon but does not include the start codon; whereas, the 3′ UTR starts immediately following the stop codon and continues until the transcriptional termination signal. There is growing body of evidence about the regulatory roles played by the UTRs in terms of stability of the nucleic acid molecule and translation. The regulatory features of a UTR can be incorporated into the polynucleotides of the present disclosure to, among other things, enhance the stability of the molecule. The specific features can also be incorporated to ensure controlled down-regulation of the transcript in case they are misdirected to undesired organs sites. A variety of 5′ UTR and 3′ UTR sequences are known and available in the art.

A 5′ UTR is region of an mRNA that is directly upstream (5′) from the start codon (the first codon of an mRNA transcript translated by a ribosome). A 5′ UTR does not encode a protein (is non-coding). Natural 5′ UTRs have features that play roles in translation initiation. They harbor signatures like Kozak sequences which are commonly known to be involved in the process by which the ribosome initiates translation of many genes. Kozak sequences have the consensus CCR(A/G)CCAUGG (SEQ ID NO: 29), where R is a purine (adenine or guanine) three bases upstream of the start codon (AUG), which is followed by another ‘G’. 5′ UTRs also have been known to form secondary structures which are involved in elongation factor binding.

In some embodiments of the disclosure, a 5′ UTR is a heterologous UTR, i.e., is a UTR found in nature associated with a different ORF. In another embodiment, a 5′ UTR is a synthetic UTR, i.e., does not occur in nature. Synthetic UTRs include UTRs that have been mutated to improve their properties, e.g., which increase gene expression as well as those which are completely synthetic. Exemplary 5′ UTRs include Xenopus or human derived a-globin or b-globin (8278063; 9012219), human cytochrome b-245 a polypeptide, and hydroxysteroid (17b) dehydrogenase, and Tobacco etch virus (US8278063, 9012219). CMV immediate-early 1 (IE1) gene (US20140206753, WO2013/185069), the sequence GGGAUCCUACC (SEQ ID NO: 30) (WO2014144196) may also be used. In another embodiment, 5′ UTR of a TOP gene is a 5′ UTR of a TOP gene lacking the 5′ TOP motif (the oligopyrimidine tract) (e.g., WO/2015101414, WO2015101415, WO/2015/062738, WO2015024667, WO2015024667; 5′ UTR element derived from ribosomal protein Large 32 (L32) gene (WO/2015101414, WO2015101415, WO/2015/062738), 5′ UTR element derived from the 5′UTR of an hydroxysteroid (17-β) dehydrogenase 4 gene (HSD17B4) (WO2015024667), or a 5′ UTR element derived from the 5′ UTR of ATP5A1 (WO2015024667) can be used. In some embodiments, an internal ribosome entry site (IRES) is used instead of a 5′ UTR.

In some embodiments, a 5′ UTR of the present disclosure comprises a sequence selected from SEQ ID NO: 3 and SEQ ID NO: 4.

A 3′ UTR is region of an mRNA that is directly downstream (3′) from the stop codon (the codon of an mRNA transcript that signals a termination of translation). A 3′ UTR does not encode a protein (is non-coding). Natural or wild type 3′ UTRs are known to have stretches of adenosines and uridines embedded in them. These AU rich signatures are particularly prevalent in genes with high rates of turnover. Based on their sequence features and functional properties, the AU rich elements (AREs) can be separated into three classes (Chen et al, 1995): Class I AREs contain several dispersed copies of an AUUUA motif within U-rich regions. C-Myc and MyoD contain class I AREs. Class II AREs possess two or more overlapping UUAUUUA(U/A)(U/A) (SEQ ID NO: 31) nonamers. Molecules containing this type of AREs include GM-CSF and TNF-a. Class III ARES are less well defined. These U rich regions do not contain an AUUUA motif. c-Jun and Myogenin are two well-studied examples of this class. Most proteins binding to the AREs are known to destabilize the messenger, whereas members of the ELAV family, most notably HuR, have been documented to increase the stability of mRNA. HuR binds to AREs of all the three classes. Engineering the HuR specific binding sites into the 3′ UTR of nucleic acid molecules will lead to HuR binding and thus, stabilization of the message in vivo.

Introduction, removal or modification of 3′ UTR AU rich elements (AREs) can be used to modulate the stability of nucleic acids (e.g., RNA) of the disclosure. When engineering specific nucleic acids, one or more copies of an ARE can be introduced to make nucleic acids of the disclosure less stable and thereby curtail translation and decrease production of the resultant protein. Likewise, AREs can be identified and removed or mutated to increase the intracellular stability and thus increase translation and production of the resultant protein. Transfection experiments can be conducted in relevant cell lines, using nucleic acids of the disclosure and protein production can be assayed at various time points post-transfection. For example, cells can be transfected with different ARE-engineering molecules and by using an ELISA kit to the relevant protein and assaying protein produced at 6 hour, 12 hour, 24 hour, 48 hour, and 7 days post-transfection.

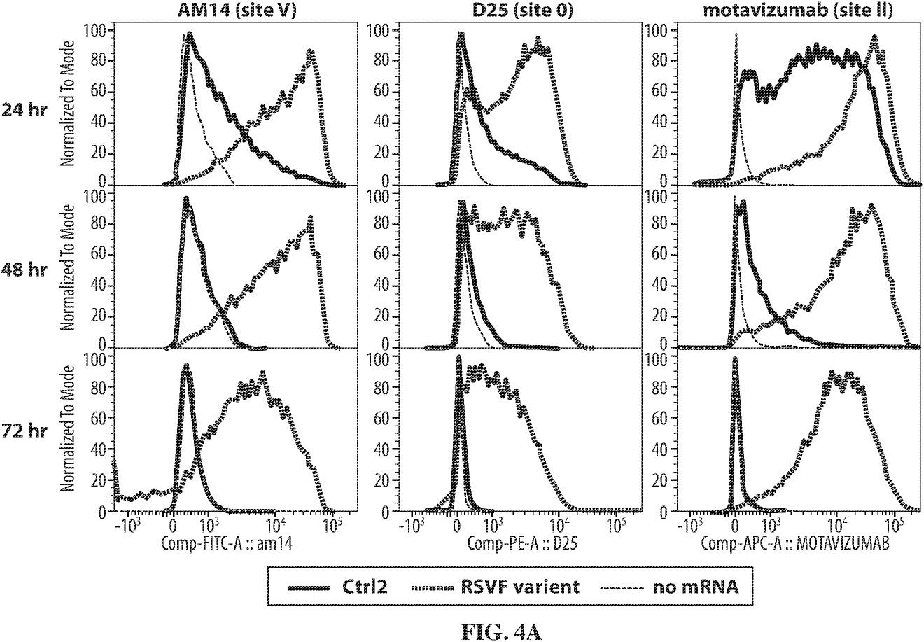

3′ UTRs may be heterologous or synthetic. With respect to 3′ UTRs, globin UTRs, including Xenopus β-globin UTRs and human β-globin UTRs are known in the art (8278063, 9012219, US20110086907). A nucleic acid (e.g., mRNA) encoding a modified β-globin with enhanced stability in some cell types by cloning two sequential human β-globin 3′ UTRs head to tail has been developed and is well known in the art (US2012/0195936, WO2014/071963). In addition, a2-globin, a1-globin, UTRs and mutants thereof are also known in the art (WO2015101415, WO2015024667). Other 3′ UTRs described in the mRNA in the non-patent literature include CYBA (Ferizi et al., 2015) and albumin (Thess et al., 2015). Other exemplary 3′ UTRs include that of bovine or human growth hormone (wild type or modified) (WO2013/185069, US20140206753, WO2014152774), rabbit β globin and hepatitis B virus (HBV), α-globin 3′ UTR and Viral VEEV 3′ UTR sequences are also known in the art. In some embodiments, the sequence UUUGAAUU (WO2014144196) is used. In some embodiments, 3′ UTRs of human and mouse ribosomal protein are used. Other examples include rps9 3′ UTR (WO2015101414), FIG. 4 (WO2015101415), and human albumin 7 (WO2015101415).

In some embodiments, a 3′ UTR of the present disclosure comprises a sequence selected from SEQ ID NO: 5 and SEQ ID NO: 6.

Those of ordinary skill in the art will understand that 5′ UTRs that are heterologous or synthetic may be used with any desired 3′ UTR sequence. For example, a heterologous 5′ UTR may be used with a synthetic 3′ UTR with a heterologous 3′ UTR.

Non-UTR sequences may also be used as regions or subregions within a nucleic acid. For example, introns or portions of introns sequences may be incorporated into regions of nucleic acid of the disclosure. Incorporation of intronic sequences may increase protein production as well as nucleic acid levels.

Combinations of features may be included in flanking regions and may be contained within other features. For example, the ORF may be flanked by a 5′ UTR which may contain a strong Kozak translational initiation signal and/or a 3′ UTR which may include an oligo(dT) sequence for templated addition of a poly-A tail. 5′ UTR may comprise a first polynucleotide fragment and a second polynucleotide fragment from the same and/or different genes such as the 5′ UTRs described in U.S. Pat. Application Publication No.20100293625 and PCT/US2014/069155, herein incorporated by reference in its entirety.

It should be understood that any UTR from any gene may be incorporated into the regions of a nucleic acid. Furthermore, multiple wild-type UTRs of any known gene may be utilized. It is also within the scope of the present disclosure to provide artificial UTRs which are not variants of wild type regions. These UTRs or portions thereof may be placed in the same orientation as in the transcript from which they were selected or may be altered in orientation or location. Hence a 5′ or 3′ UTR may be inverted, shortened, lengthened, made with one or more other 5′ UTRs or 3′ UTRs. As used herein, the term “altered” as it relates to a UTR sequence, means that the UTR has been changed in some way in relation to a reference sequence. For example, a 3′ UTR or 5′ UTR may be altered relative to a wild-type or native UTR by the change in orientation or location as taught above or may be altered by the inclusion of additional nucleotides, deletion of nucleotides, swapping or transposition of nucleotides. Any of these changes producing an “altered” UTR (whether 3′ or 5′) comprise a variant UTR.

In some embodiments, a double, triple or quadruple UTR such as a 5′ UTR or 3′ UTR may be used. As used herein, a “double” UTR is one in which two copies of the same UTR are encoded either in series or substantially in series. For example, a double beta-globin 3′ UTR may be used as described in U.S. Pat. Publication 20100129877, the contents of which are incorporated herein by reference in its entirety.

It is also within the scope of the present disclosure to have patterned UTRs. As used herein “patterned UTRs” are those UTRs which reflect a repeating or alternating pattern, such as ABABAB or AABBAABBAABB or ABCABCABC or variants thereof repeated once, twice, or more than 3 times. In these patterns, each letter, A, B, or C represent a different UTR at the nucleotide level.

In some embodiments, flanking regions are selected from a family of transcripts whose proteins share a common function, structure, feature or property. For example, polypeptides of interest may belong to a family of proteins which are expressed in a particular cell, tissue or at some time during development. The UTRs from any of these genes may be swapped for any other UTR of the same or different family of proteins to create a new polynucleotide. As used herein, a “family of proteins” is used in the broadest sense to refer to a group of two or more polypeptides of interest which share at least one function, structure, feature, localization, origin, or expression pattern.

The untranslated region may also include translation enhancer elements (TEE). As a non-limiting example, the TEE may include those described in U.S. Application No.20090226470, herein incorporated by reference in its entirety, and those known in the art.

### In Vitro Transcription of RNA

cDNA encoding the polynucleotides described herein may be transcribed using an in vitro transcription (IVT) system. In vitro transcription of RNA is known in the art and is described in International Publication WO/2014/152027, which is incorporated by reference herein in its entirety.

In some embodiments, the RNA transcript is generated using a non-amplified, linearized DNA template in an in vitro transcription reaction to generate the RNA transcript. In some embodiments, the template DNA is isolated DNA. In some embodiments, the template DNA is cDNA. In some embodiments, the cDNA is formed by reverse transcription of a RNA polynucleotide, for example, but not limited to respiratory virus mRNA. In some embodiments, cells, e.g., bacterial cells, e.g., E. coli, e.g., DH-1 cells are transfected with the plasmid DNA template. In some embodiments, the transfected cells are cultured to replicate the plasmid DNA which is then isolated and purified. In some embodiments, the DNA template includes a RNA polymerase promoter, e.g., a T7 promoter located 5′ to and operably linked to the gene of interest.

In some embodiments, an in vitro transcription template encodes a 5′ untranslated (UTR) region, contains an open reading frame, and encodes a 3′ UTR and a poly(A) tail. The particular nucleic acid sequence composition and length of an in vitro transcription template will depend on the mRNA encoded by the template.

A “5′ untranslated region” (UTR) refers to a region of an mRNA that is directly upstream (i.e., 5′) from the start codon (i.e., the first codon of an mRNA transcript translated by a ribosome) that does not encode a polypeptide. When RNA transcripts are being generated, the 5′ UTR may comprise a promoter sequence. Such promoter sequences are known in the art. It should be understood that such promoter sequences will not be present in a vaccine of the disclosure.

A “3′ untranslated region” (UTR) refers to a region of an mRNA that is directly downstream (i.e., 3′) from the stop codon (i.e., the codon of an mRNA transcript that signals a termination of translation) that does not encode a polypeptide.

An “open reading frame” is a continuous stretch of DNA beginning with a start codon (e.g., methionine (ATG)), and ending with a stop codon (e.g., TAA, TAG or TGA) and encodes a polypeptide.

A “poly(A) tail” is a region of mRNA that is downstream, e.g., directly downstream (i.e., 3′), from the 3′ UTR that contains multiple, consecutive adenosine monophosphates. A poly(A) tail may contain 10 to 300 adenosine monophosphates. For example, a poly(A) tail may contain 10, 20, 30, 40, 50, 60, 70, 80, 90, 100, 110, 120, 130, 140, 150, 160, 170, 180, 190, 200, 210, 220, 230, 240, 250, 260, 270, 280, 290 or 300 adenosine monophosphates. In some embodiments, a poly(A) tail contains 50 to 250 adenosine monophosphates. In a relevant biological setting (e.g., in cells, in vivo) the poly(A) tail functions to protect mRNA from enzymatic degradation, e.g., in the cytoplasm, and aids in transcription termination, and/or export of the mRNA from the nucleus and translation.

In some embodiments, a nucleic acid includes 200 to 3,000 nucleotides. For example, a nucleic acid may include 200 to 500, 200 to 1000, 200 to 1500, 200 to 3000, 500 to 1000, 500 to 1500, 500 to 2000, 500 to 3000, 1000 to 1500, 1000 to 2000, 1000 to 3000, 1500 to 3000, or 2000 to 3000 nucleotides).

An in vitro transcription system typically comprises a transcription buffer, nucleotide triphosphates (NTPs), an RNase inhibitor and a polymerase.

The NTPs may be manufactured in house, may be selected from a supplier, or may be synthesized as described herein. The NTPs may be selected from, but are not limited to, those described herein including natural and unnatural (modified) NTPs.

Any number of RNA polymerases or variants may be used in the method of the present disclosure. The polymerase may be selected from, but is not limited to, a phage RNA polymerase, e.g., a T7 RNA polymerase, a T3 RNA polymerase, a SP6 RNA polymerase, and/or mutant polymerases such as, but not limited to, polymerases able to incorporate modified nucleic acids and/or modified nucleotides, including chemically modified nucleic acids and/or nucleotides. Some embodiments exclude the use of DNase.

In some embodiments, the RNA transcript is capped via enzymatic capping. In some embodiments, the RNA comprises 5′ terminal cap, for example, 7mG(5′)ppp(5′)NlmpNp.

### Chemical Synthesis

Solid-phase chemical synthesis. Nucleic acids the present disclosure may be manufactured in whole or in part using solid phase techniques. Solid-phase chemical synthesis of nucleic acids is an automated method wherein molecules are immobilized on a solid support and synthesized step by step in a reactant solution. Solid-phase synthesis is useful in site-specific introduction of chemical modifications in the nucleic acid sequences.

Liquid Phase Chemical Synthesis. The synthesis of nucleic acids of the present disclosure by the sequential addition of monomer building blocks may be carried out in a liquid phase.

Combination of Synthetic Methods. The synthetic methods discussed above each has its own advantages and limitations. Attempts have been conducted to combine these methods to overcome the limitations. Such combinations of methods are within the scope of the present disclosure. The use of solid-phase or liquid-phase chemical synthesis in combination with enzymatic ligation provides an efficient way to generate long chain nucleic acids that cannot be obtained by chemical synthesis alone.

### Ligation of Nucleic Acid Regions or Subregions

Assembling nucleic acids by a ligase may also be used. DNA or RNA ligases promote intermolecular ligation of the 5′ and 3′ ends of polynucleotide chains through the formation of a phosphodiester bond. Nucleic acids such as chimeric polynucleotides and/or circular nucleic acids may be prepared by ligation of one or more regions or subregions. DNA fragments can be joined by a ligase catalyzed reaction to create recombinant DNA with different functions. Two oligodeoxynucleotides, one with a 5′ phosphoryl group and another with a free 3′ hydroxyl group, serve as substrates for a DNA ligase.

### Purification

Purification of the nucleic acids described herein may include, but is not limited to, nucleic acid clean-up, quality assurance and quality control. Clean-up may be performed by methods known in the arts such as, but not limited to, AGENCOURT® beads (Beckman Coulter Genomics, Danvers, MA), poly-T beads, LNA™ oligo-T capture probes (EXIQON® Inc, Vedbaek, Denmark) or HPLC based purification methods such as, but not limited to, strong anion exchange HPLC, weak anion exchange HPLC, reverse phase HPLC (RP-HPLC), and hydrophobic interaction HPLC (HIC-HPLC). The term “purified” when used in relation to a nucleic acid such as a “purified nucleic acid” refers to one that is separated from at least one contaminant. A “contaminant” is any substance that makes another unfit, impure or inferior. Thus, a purified nucleic acid (e.g., DNA and RNA) is present in a form or setting different from that in which it is found in nature, or a form or setting different from that which existed prior to subjecting it to a treatment or purification method.

A quality assurance and/or quality control check may be conducted using methods such as, but not limited to, gel electrophoresis, UV absorbance, or analytical HPLC.

In some embodiments, the nucleic acids may be sequenced by methods including, but not limited to reverse-transcriptase-PCR.

### Quantification

In some embodiments, the nucleic acids of the present disclosure may be quantified in exosomes or when derived from one or more bodily fluid. Bodily fluids include peripheral blood, serum, plasma, ascites, urine, cerebrospinal fluid (CSF), sputum, saliva, bone marrow, synovial fluid, aqueous humor, amniotic fluid, cerumen, breast milk, broncheoalveolar lavage fluid, semen, prostatic fluid, cowper’s fluid or pre-ejaculatory fluid, sweat, fecal matter, hair, tears, cyst fluid, pleural and peritoneal fluid, pericardial fluid, lymph, chyme, chyle, bile, interstitial fluid, menses, pus, sebum, vomit, vaginal secretions, mucosal secretion, stool water, pancreatic juice, lavage fluids from sinus cavities, bronchopulmonary aspirates, blastocyl cavity fluid, and umbilical cord blood. Alternatively, exosomes may be retrieved from an organ selected from the group consisting of lung, heart, pancreas, stomach, intestine, bladder, kidney, ovary, testis, skin, colon, breast, prostate, brain, esophagus, liver, and placenta.

Assays may be performed using antigen-specific probes, cytometry, qRT-PCR, real-time PCR, PCR, flow cytometry, electrophoresis, mass spectrometry, or combinations thereof while the exosomes may be isolated using immunohistochemical methods such as enzyme linked immunosorbent assay (ELISA) methods. Exosomes may also be isolated by size exclusion chromatography, density gradient centrifugation, differential centrifugation, nanomembrane ultrafiltration, immunosorbent capture, affinity purification, microfluidic separation, or combinations thereof.

These methods afford the investigator the ability to monitor, in real time, the level of nucleic acids remaining or delivered. This is possible because the nucleic acids of the present disclosure, in some embodiments, differ from the endogenous forms due to the structural or chemical modifications.

In some embodiments, the nucleic acid may be quantified using methods such as, but not limited to, ultraviolet visible spectroscopy (UV/Vis). A non-limiting example of a UV/Vis spectrometer is a NANODROP® spectrometer (ThermoFisher, Waltham, MA). The quantified nucleic acid may be analyzed in order to determine if the nucleic acid may be of proper size, check that no degradation of the nucleic acid has occurred. Degradation of the nucleic acid may be checked by methods such as, but not limited to, agarose gel electrophoresis, HPLC based purification methods such as, but not limited to, strong anion exchange HPLC, weak anion exchange HPLC, reverse phase HPLC (RP-HPLC), and hydrophobic interaction HPLC (HIC-HPLC), liquid chromatography-mass spectrometry (LCMS), capillary electrophoresis (CE) and capillary gel electrophoresis (CGE).

### Lipid Nanoparticles (LNPs)

In some embodiments, the RNA (e.g., mRNA) of the disclosure is formulated in a lipid nanoparticle (LNP). Lipid nanoparticles typically comprise ionizable cationic lipid, non-cationic lipid, sterol and PEG lipid components along with the nucleic acid cargo of interest. The lipid nanoparticles of the disclosure can be generated using components, compositions, and methods as are generally known in the art, see for example PCT/US2016/052352; PCT/US2016/068300; PCT/US2017/037551; PCT/US2015/027400; PCT/US2016/047406; PCT/US2016000129; PCT/US2016/014280; PCT/US2016/014280; PCT/US2017/038426; PCT/US2014/027077; PCT/US2014/055394; PCT/US2016/52117; PCT/US2012/069610; PCT/US2017/027492; PCT/US2016/059575 and PCT/US2016/069491 all of which are incorporated by reference herein in their entirety.

Vaccines of the present disclosure are typically formulated in lipid nanoparticle. In some embodiments, the lipid nanoparticle comprises at least one ionizable cationic lipid, at least one non-cationic lipid, at least one sterol, and/or at least one polyethylene glycol (PEG)-modified lipid.

In some embodiments, the lipid nanoparticle comprises a molar ratio of 20-60% ionizable cationic lipid. For example, the lipid nanoparticle may comprise a molar ratio of 20-50%, 20-40%, 20-30%, 30-60%, 30-50%, 30-40%, 40-60%, 40-50%, or 50-60% ionizable cationic lipid. In some embodiments, the lipid nanoparticle comprises a molar ratio of 20%, 30%, 40%, 50, or 60% ionizable cationic lipid.

In some embodiments, the lipid nanoparticle comprises a molar ratio of 5-25% non-cationic lipid. For example, the lipid nanoparticle may comprise a molar ratio of 5-20%, 5-15%, 5-10%, 10-25%, 10-20%, 10-25%, 15-25%, 15-20%, or 20-25% non-cationic lipid. In some embodiments, the lipid nanoparticle comprises a molar ratio of 5%, 10%, 15%, 20%, or25% non-cationic lipid.

In some embodiments, the lipid nanoparticle comprises a molar ratio of 25-55% sterol. For example, the lipid nanoparticle may comprise a molar ratio of 25-50%, 25-45%, 25-40%, 25-35%, 25-30%, 30-55%, 30-50%, 30-45%, 30-40%, 30-35%, 35-55%, 35-50%, 35-45%, 35-40%, 40-55%, 40-50%, 40-45%, 45-55%, 45-50%, or 50-55% sterol. In some embodiments, the lipid nanoparticle comprises a molar ratio of 25%, 30%, 35%, 40%, 45%, 50%, or 55% sterol.

In some embodiments, the lipid nanoparticle comprises a molar ratio of 0.5-15% PEG-modified lipid. For example, the lipid nanoparticle may comprise a molar ratio of 0.5-10%, 0.5-5%, 1-15%, 1-10%, 1-5%, 2-15%, 2-10%, 2-5%, 5-15%, 5-10%, or 10-15%. In some embodiments, the lipid nanoparticle comprises a molar ratio of 0.5%, 1%, 2%, 3%, 4%, 5%, 6%, 7%, 8%, 9%, 10%, 11%, 12%, 13%, 14%, or 15% PEG-modified lipid.

In some embodiments, the lipid nanoparticle comprises a molar ratio of 20-60% ionizable cationic lipid, 5-25% non-cationic lipid, 25-55% sterol, and 0.5-15% PEG-modified lipid.

In some embodiments, an ionizable cationic lipid of the disclosure comprises a compound of Formula (I):

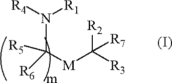

or a salt or isomer thereof, wherein:
R₁ is selected from the group consisting of C₅₋₃₀ alkyl, C₅₋₂₀ alkenyl, -R*YR”, -YR”, and -R″M′R′;
R₂ and R₃ are independently selected from the group consisting of H, C₁₋₁₄ alkyl, C₂₋₁₄ alkenyl, -R*YR”, -YR”, and -R*OR”, or R₂ and R₃, together with the atom to which they are attached, form a heterocycle or carbocycle;
R₄ is selected from the group consisting of a C₃₋₆ carbocycle, —(CH₂)ₙQ, -(CH₂)ₙCHQR, -CHQR, -CQ(R)₂, and unsubstituted C₁₋₆ alkyl, where Q is selected from a carbocycle, heterocycle, -OR, -O(CH₂)ₙN(R)₂, —C(O)OR, —OC(O)R, -CX₃, -CX₂H, -CXH₂, —CN, -N(R)₂, -C(O)N(R)₂, -N(R)C(O)R, -N(R)S(O)₂R, -N(R)C(O)N(R)₂, -N(R)C(S)N(R)₂, -N(R)R₈, —O(CH₂)ₙOR, -N(R)C(=NR₉)N(R)₂, -N(R)C(=CHR₉)N(R)₂, -OC(O)N(R)₂, -N(R)C(O)OR, —N(OR)C(O)R, —N(OR)S(O)₂R, —N(OR)C(O)OR, -N(OR)C(O)N(R)₂, -N(OR)C(S)N(R)₂, —N(OR)C(═NR₉)N(R)₂, -N(OR)C(=CHR₉)N(R)₂, —C(═NR₉)N(R)₂, —C(═NR₉)R, -C(O)N(R)OR, and -C(R)N(R)₂C(O)OR, and each n is independently selected from 1, 2, 3, 4, and 5;
each R₅ is independently selected from the group consisting of C₁₋₃ alkyl, C₂₋₃ alkenyl, and H;
each R₆ is independently selected from the group consisting of C₁₋₃ alkyl, C₂₋₃ alkenyl, and H;
M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group;
R₇ is selected from the group consisting of C₁₋₃ alkyl, C₂₋₃ alkenyl, and H;
R₈ is selected from the group consisting of C₃₋₆ carbocycle and heterocycle;
R₉ is selected from the group consisting of H, CN, NO₂, C₁₋₆ alkyl, -OR, —S(O)₂R, -S(O)₂N(R)₂, C₂₋₆ alkenyl, C₃₋₆ carbocycle and heterocycle;
each R is independently selected from the group consisting of C₁₋₃ alkyl, C₂₋₃ alkenyl, and H;
each R′ is independently selected from the group consisting of C₁₋₁₈ alkyl, C₂₋₁₈ alkenyl, -R*YR”, -YR”, and H;
each R″ is independently selected from the group consisting of C₃₋₁₄ alkyl and C₃₋₁₄ alkenyl;
each R* is independently selected from the group consisting of C₁₋₁₂ alkyl and C₂₋₁₂ alkenyl;
each Y is independently a C₃₋₆ carbocycle;
each X is independently selected from the group consisting of F, Cl, Br, and I; and
m is selected from 5, 6, 7, 8, 9, 10, 11, 12, and 13.

In some embodiments, a subset of compounds of Formula (I) includes those in which when R₄ is —(CH₂)ₙQ, -(CH₂)ₙCHQR, -CHQR, or -CQ(R)₂, then (i) Q is not -N(R)₂ when n is 1, 2, 3, 4 or 5, or (ii) Q is not 5, 6, or 7-membered heterocycloalkyl when n is 1 or 2.

In some embodiments, another subset of compounds of Formula (I) includes those in which
R₁ is selected from the group consisting of C₅₋₃₀ alkyl, C₅₋₂₀ alkenyl, -R*YR”, -YR”, and -R″M′R′;
R₂ and R₃ are independently selected from the group consisting of H, C₁₋₁₄ alkyl, C₂₋₁₄ alkenyl, -R*YR”, -YR”, and -R*OR”, or R₂ and R₃, together with the atom to which they are attached, form a heterocycle or carbocycle;
R₄ is selected from the group consisting of a C₃₋₆ carbocycle, —(CH₂)ₙQ, —(CH₂)ₙCHQR, —CHQR, —CQ(R)₂, and unsubstituted C₁₋₆ alkyl, where Q is selected from a C₃₋₆ carbocycle, a 5-to 14-membered heteroaryl having one or more heteroatoms selected from N, O, and S, -OR, -O(CH₂)ₙN(R)₂, —C(O)OR, —OC(O)R, -CX₃, -CX₂H, -CXH₂, —CN, —C(O)N(R)₂, —N(R)C(O)R, —N(R)S(O)₂R, —N(R)C(O)N(R)₂, —N(R)C(S)N(R)₂, —CRN(R)₂C(O)OR, -N(R)R₈, —O(CH₂)ₙOR, -N(R)C(=NR₉)N(R)₂, —N(R)C(═CHR₉)N(R)₂, —OC(O)N(R)₂, -N(R)C(O)OR, —N(OR)C(O)R, —N(OR)S(O)₂R, —N(OR)C(O)OR, -N(OR)C(O)N(R)₂, -N(OR)C(S)N(R)₂, —N(OR)C(═NR₉)N(R)₂, -N(OR)C(=CHR₉)N(R)₂, —C(═NR₉)N(R)₂, —C(═NR₉)R, -C(O)N(R)OR, and a 5- to 14-membered heterocycloalkyl having one or more heteroatoms selected from N, O, and S which is substituted with one or more substituents selected from oxo (═O), OH, amino, mono- or di-alkylamino, and C₁₋₃ alkyl, and each n is independently selected from 1, 2, 3, 4, and 5;
each R₅ is independently selected from the group consisting of C₁₋₃ alkyl, C₂₋₃ alkenyl, and H;
each R₆ is independently selected from the group consisting of C₁₋₃ alkyl, C₂₋₃ alkenyl, and H;
M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group;
R₇ is selected from the group consisting of C₁₋₃ alkyl, C₂₋₃ alkenyl, and H;
R₈ is selected from the group consisting of C₃₋₆ carbocycle and heterocycle;
R₉ is selected from the group consisting of H, CN, NO₂, C₁₋₆ alkyl, -OR, —S(O)₂R, -S(O)₂N(R)₂, C₂₋₆ alkenyl, C₃₋₆ carbocycle and heterocycle;
each R is independently selected from the group consisting of C₁₋₃ alkyl, C₂₋₃ alkenyl, and H;
each R′ is independently selected from the group consisting of C₁₋₁₈ alkyl, C₂₋₁₈ alkenyl, -R*YR”, -YR”, and H;
each R″ is independently selected from the group consisting of C₃₋₁₄ alkyl and C₃₋₁₄ alkenyl;
each R* is independently selected from the group consisting of C₁₋₁₂ alkyl and C₂₋₁₂ alkenyl;
each Y is independently a C₃₋₆ carbocycle;
each X is independently selected from the group consisting of F, Cl, Br, and I; and
m is selected from 5, 6, 7, 8, 9, 10, 11, 12, and 13,
or salts or isomers thereof.

In some embodiments, another subset of compounds of Formula (I) includes those in which
R₁ is selected from the group consisting of C₅₋₃₀ alkyl, C₅₋₂₀ alkenyl, -R*YR”, -YR”, and -R″M′R′;
R₂ and R₃ are independently selected from the group consisting of H, C₁₋₁₄ alkyl, C₂₋₁₄ alkenyl, -R*YR”, -YR”, and -R*OR”, or R₂ and R₃, together with the atom to which they are attached, form a heterocycle or carbocycle;
R₄ is selected from the group consisting of a C₃₋₆ carbocycle, —(CH₂)ₙQ, -(CH₂)ₙCHQR, -CHQR, -CQ(R)₂, and unsubstituted C₁₋₆ alkyl, where Q is selected from a C₃₋₆ carbocycle, a 5-to 14-membered heterocycle having one or more heteroatoms selected from N, O, and S, -OR, -O(CH₂)ₙN(R)₂, —C(O)OR, —OC(O)R, -CX₃, -CX₂H, -CXH₂, —CN, -C(O)N(R)₂, -N(R)C(O)R, -N(R)S(O)₂R, -N(R)C(O)N(R)₂, -N(R)C(S)N(R)₂, -CRN(R)₂C(O)OR, -N(R)R₈, —O(CH₂)ₙOR, -N(R)C(=NR₉)N(R)₂, -N(R)C(=CHR₉)N(R)₂, -OC(O)N(R)₂, -N(R)C(O)OR, —N(OR)C(O)R, —N(OR)S(O)₂R, —N(OR)C(O)OR, -N(OR)C(O)N(R)₂, -N(OR)C(S)N(R)₂, —N(OR)C(═NR₉)N(R)₂, -N(OR)C(=CHR₉)N(R)₂, —C(═NR₉)R, -C(O)N(R)OR, and —C(═NR₉)N(R)₂, and each n is independently selected from 1, 2, 3, 4, and 5; and when Q is a 5- to 14-membered heterocycle and (i) R₄ is —(CH₂)ₙQ in which n is 1 or 2, or (ii) R₄ is -(CH₂)ₙCHQR in which n is 1, or (iii) R₄ is -CHQR, and -CQ(R)₂, then Q is either a 5- to 14-membered heteroaryl or 8- to 14-membered heterocycloalkyl;
each R₅ is independently selected from the group consisting of C₁₋₃ alkyl, C₂₋₃ alkenyl, and H;
each R₆ is independently selected from the group consisting of C₁₋₃ alkyl, C₂₋₃ alkenyl, and H;
M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group;
R₇ is selected from the group consisting of C₁₋₃ alkyl, C₂₋₃ alkenyl, and H;
R₈ is selected from the group consisting of C₃₋₆ carbocycle and heterocycle;
R₉ is selected from the group consisting of H, CN, NO₂, C₁₋₆ alkyl, -OR, —S(O)₂R, -S(O)₂N(R)₂, C₂₋₆ alkenyl, C₃₋₆ carbocycle and heterocycle;
each R is independently selected from the group consisting of C₁₋₃ alkyl, C₂₋₃ alkenyl, and H;
each R′ is independently selected from the group consisting of C₁₋₁₈ alkyl, C₂₋₁₈ alkenyl, -R*YR”, -YR”, and H;
each R″ is independently selected from the group consisting of C₃₋₁₄ alkyl and C₃₋₁₄ alkenyl;
each R* is independently selected from the group consisting of C₁₋₁₂ alkyl and C₂₋₁₂ alkenyl;
each Y is independently a C₃₋₆ carbocycle;
each X is independently selected from the group consisting of F, Cl, Br, and I; and
m is selected from 5, 6, 7, 8, 9, 10, 11, 12, and 13,
or salts or isomers thereof.

In some embodiments, another subset of compounds of Formula (I) includes those in which
R₁ is selected from the group consisting of C₅₋₃₀ alkyl, C₅₋₂₀ alkenyl, -R*YR”, -YR”, and -R″M′R′;
R₂ and R₃ are independently selected from the group consisting of H, C₁₋₁₄ alkyl, C₂₋₁₄ alkenyl, -R*YR”, -YR″, and -R*OR”, or R₂ and R₃, together with the atom to which they are attached, form a heterocycle or carbocycle;
R₄ is selected from the group consisting of a C₃₋₆ carbocycle, —(CH₂)ₙQ, -(CH₂)ₙCHQR, -CHQR, -CQ(R)₂, and unsubstituted C₁₋₆ alkyl, where Q is selected from a C₃₋₆ carbocycle, a 5-to 14-membered heteroaryl having one or more heteroatoms selected from N, O, and S, -OR, -O(CH₂)ₙN(R)₂, —C(O)OR, —OC(O)R, -CX₃, -CX₂H, -CXH₂, —CN, -C(O)N(R)₂, -N(R)C(O)R, -N(R)S(O)₂R, -N(R)C(O)N(R)₂, -N(R)C(S)N(R)₂, -CRN(R)₂C(O)OR, -N(R)R₈, —O(CH₂)ₙOR, -N(R)C(=NR₉)N(R)₂, -N(R)C(=CHR₉)N(R)₂, -OC(O)N(R)₂, -N(R)C(O)OR, —N(OR)C(O)R, —N(OR)S(O)₂R, —N(OR)C(O)OR, -N(OR)C(O)N(R)₂, -N(OR)C(S)N(R)₂, —N(OR)C(═NR₉)N(R)₂, -N(OR)C(=CHR₉)N(R)₂, —C(═NR₉)R, -C(O)N(R)OR, and —C(═NR₉)N(R)₂, and each n is independently selected from 1, 2, 3, 4, and 5;
each R₅ is independently selected from the group consisting of C₁₋₃ alkyl, C₂₋₃ alkenyl, and H;
each R₆ is independently selected from the group consisting of C₁₋₃ alkyl, C₂₋₃ alkenyl, and H;
M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group;
R₇ is selected from the group consisting of C₁₋₃ alkyl, C₂₋₃ alkenyl, and H;
R₈ is selected from the group consisting of C₃₋₆ carbocycle and heterocycle;
R₉ is selected from the group consisting of H, CN, NO₂, C₁₋₆ alkyl, -OR, —S(O)₂R, -S(O)₂N(R)₂, C₂₋₆ alkenyl, C₃₋₆ carbocycle and heterocycle;
each R is independently selected from the group consisting of C₁₋₃ alkyl, C₂₋₃ alkenyl, and H;
each R′ is independently selected from the group consisting of C₁₋₁₈ alkyl, C₂₋₁₈ alkenyl, -R*YR”, -YR”, and H;
each R″ is independently selected from the group consisting of C₃₋₁₄ alkyl and C₃₋₁₄ alkenyl;
each R* is independently selected from the group consisting of C₁₋₁₂ alkyl and C₂₋₁₂ alkenyl;
each Y is independently a C₃₋₆ carbocycle;
each X is independently selected from the group consisting of F, Cl, Br, and I; and
m is selected from 5, 6, 7, 8, 9, 10, 11, 12, and 13,
or salts or isomers thereof.

In some embodiments, another subset of compounds of Formula (I) includes those in which
R₁ is selected from the group consisting of C₅₋₃₀ alkyl, C₅₋₂₀ alkenyl, -R*YR”, -YR”, and -R″M′R′;
R₂ and R₃ are independently selected from the group consisting of H, C₂₋₁₄ alkyl, C₂₋₁₄ alkenyl, -R*YR”, -YR”, and -R*OR”, or R₂ and R₃, together with the atom to which they are attached, form a heterocycle or carbocycle;
R₄ is —(CH₂)ₙQ or -(CH₂)ₙCHQR, where Q is -N(R)₂, and n is selected from 3, 4, and 5;
each R₅ is independently selected from the group consisting of C₁₋₃ alkyl, C₂₋₃ alkenyl, and H;
each R₆ is independently selected from the group consisting of C₁₋₃ alkyl, C₂₋₃ alkenyl, and H;
M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group;
R₇ is selected from the group consisting of C₁₋₃ alkyl, C₂₋₃ alkenyl, and H;
each R is independently selected from the group consisting of C₁₋₃ alkyl, C₂₋₃ alkenyl, and H;
each R′ is independently selected from the group consisting of C₁₋₁₈ alkyl, C₂₋₁₈ alkenyl, -R*YR”, -YR”, and H;
each R″ is independently selected from the group consisting of C₃₋₁₄ alkyl and C₃₋₁₄ alkenyl;
each R* is independently selected from the group consisting of C₁₋₁₂ alkyl and C₁₋₁₂ alkenyl;
each Y is independently a C₃₋₆ carbocycle;
each X is independently selected from the group consisting of F, Cl, Br, and I; and
m is selected from 5, 6, 7, 8, 9, 10, 11, 12, and 13,
or salts or isomers thereof.

In some embodiments, another subset of compounds of Formula (I) includes those in which
R₁ is selected from the group consisting of C₅₋₃₀ alkyl, C₅₋₂₀ alkenyl, -R*YR”, -YR”, and -R″M′R′;
R₂ and R₃ are independently selected from the group consisting of C₁₋₁₄ alkyl, C₂₋₁₄ alkenyl, -R*YR”, -YR”, and -R*OR”, or R₂ and R₃, together with the atom to which they are attached, form a heterocycle or carbocycle;
R₄ is selected from the group consisting of —(CH₂)ₙQ, -(CH₂)ₙCHQR, -CHQR, and -CQ(R)₂, where Q is -N(R)₂, and n is selected from 1, 2, 3, 4, and 5;
each R₅ is independently selected from the group consisting of C₁₋₃ alkyl, C₂₋₃ alkenyl, and H;
each R₆ is independently selected from the group consisting of C₁₋₃ alkyl, C₂₋₃ alkenyl, and H;
M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group;
R₇ is selected from the group consisting of C₁₋₃ alkyl, C₂₋₃ alkenyl, and H;
each R is independently selected from the group consisting of C₁₋₃ alkyl, C₂₋₃ alkenyl, and H;
each R′ is independently selected from the group consisting of C₁₋₁₈ alkyl, C₂₋₁₈ alkenyl, -R*YR”, -YR”, and H;
each R″ is independently selected from the group consisting of C₃₋₁₄ alkyl and C₃₋₁₄ alkenyl;
each R* is independently selected from the group consisting of C₁₋₁₂ alkyl and C₁₋₁₂ alkenyl;
each Y is independently a C₃₋₆ carbocycle;
each X is independently selected from the group consisting of F, Cl, Br, and I; and
m is selected from 5, 6, 7, 8, 9, 10, 11, 12, and 13,
or salts or isomers thereof.

In some embodiments, a subset of compounds of Formula (I) includes those of Formula (IA):

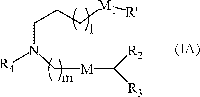

or a salt or isomer thereof, wherein 1 is selected from 1, 2, 3, 4, and 5; m is selected from 5, 6, 7, 8, and 9; M₁ is a bond or M′; R₄ is unsubstituted C₁₋₃ alkyl, or —(CH₂)_{nQ}, in which Q is OH, -NHC(S)N(R)₂, -NHC(O)N(R)₂, -N(R)C(O)R, -N(R)S(O)₂R, -N(R)R₈, -NHC(=NR₉)N(R)₂, -NHC(=CHR₉)N(R)₂, -OC(O)N(R)₂, -N(R)C(O)OR, heteroaryl or heterocycloalkyl; M and M′ are independently selected from —C(O)O—, —OC(O)—, —C(O)N(R′)—, —P(O)(OR′)O—, —S—S—, an aryl group, and a heteroaryl group; and R₂ and R₃ are independently selected from the group consisting of H, C₁₋₁₄ alkyl, and C₂₋₁₄ alkenyl.

In some embodiments, a subset of compounds of Formula (I) includes those of Formula (II):

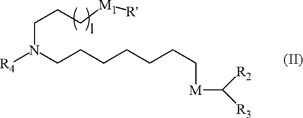

or a salt or isomer thereof, wherein 1 is selected from 1, 2, 3, 4, and 5; M₁ is a bond or M′; R₄ is unsubstituted C₁₋₃ alkyl, or —(CH₂)_{nQ}, in which n is 2, 3, or 4, and Q is OH, -NHC(S)N(R)₂, -NHC(O)N(R)₂, -N(R)C(O)R, -N(R)S(O)₂R, -N(R)R₈, -NHC(=NR₉)N(R)₂, -NHC(=CHR₉)N(R)₂, -OC(O)N(R)₂, -N(R)C(O)OR, heteroaryl or heterocycloalkyl; M and M′ are independently selected from —C(O)O—, —OC(O)—, —C(O)N(R′)—, —P(O)(OR′)O—, —S—S—, an aryl group, and a heteroaryl group; and R₂ and R₃ are independently selected from the group consisting of H, C₁₋₁₄ alkyl, and C₂₋₁₄ alkenyl.

In some embodiments, a subset of compounds of Formula (I) includes those of Formula (IIa), (IIb), (IIc), or (IIe):

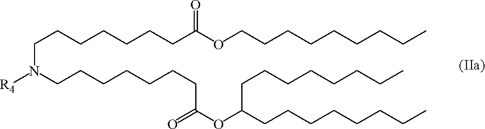

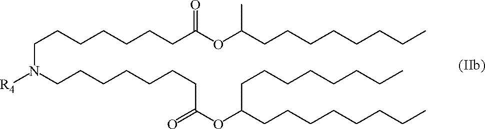

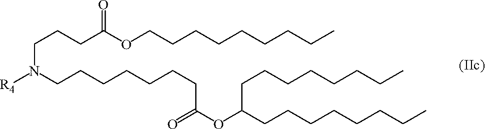

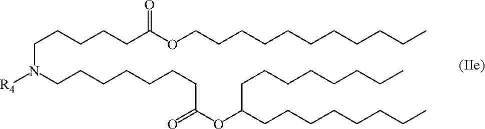

or a salt or isomer thereof, wherein R₄ is as described herein.

In some embodiments, a subset of compounds of Formula (I) includes those of Formula (IId):

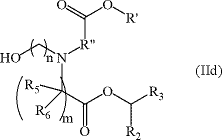

or a salt or isomer thereof, wherein n is 2, 3, or 4; and m, R′, R″, and R₂ through R₆ are as described herein. For example, each of R₂ and R₃ may be independently selected from the group consisting of C₅₋₁₄ alkyl and C₅₋₁₄ alkenyl.

In some embodiments, an ionizable cationic lipid of the disclosure comprises a compound having structure:

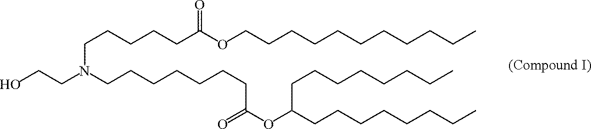 [SMILES: CCCCCCCCCCCOC(=O)CCCCCN(CCO)CCCCCCCC(=O)OC(CCCCCCCC)CCCCCCCC]

In some embodiments, an ionizable cationic lipid of the disclosure comprises a compound having structure:

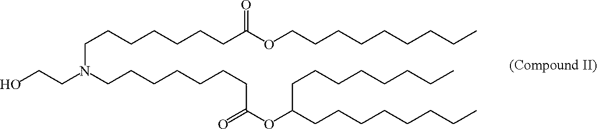 [SMILES: CCCCCCCCCOC(=O)CCCCCCCN(CCO)CCCCCCCC(=O)OC(CCCCCCCC)CCCCCCCC]

In some embodiments, a non-cationic lipid of the disclosure comprises 1,2-distearoyl-sn-glycero-3-phosphocholine (DSPC), 1,2-dioleoyl-sn-glycero-3-phosphoethanolamine (DOPE), 1,2-dilinoleoyl-sn-glycero-3-phosphocholine (DLPC), 1,2-dimyristoyl-sn-gly cero-phosphocholine (DMPC), 1,2-dioleoyl-sn-glycero-3-phosphocholine (DOPC), 1,2-dipalmitoyl-sn-glycero-3-phosphocholine (DPPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2 cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine,1,2-diarachidonoyl-sn-glycero-3-phosphocholine, 1,2-didocosahexaenoyl-sn-glycero-3-phosphocholine, 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), sphingomyelin, and mixtures thereof.

In some embodiments, a PEG modified lipid of the disclosure comprises a PEG-modified phosphatidylethanolamine, a PEG-modified phosphatidic acid, a PEG-modified ceramide, a PEG-modified dialkylamine, a PEG-modified diacylglycerol, a PEG-modified dialkylglycerol, and mixtures thereof. In some embodiments, the PEG-modified lipid is DMG-PEG, PEG-c-DOMG (also referred to as PEG-DOMG), PEG-DSG and/or PEG-DPG.

In some embodiments, a sterol of the disclosure comprises cholesterol, fecosterol, sitosterol, ergosterol, campesterol, stigmasterol, brassicasterol, tomatidine, ursolic acid, alpha-tocopherol, and mixtures thereof.

In some embodiments, a LNP of the disclosure comprises an ionizable cationic lipid of Compound 1, wherein the non-cationic lipid is DSPC, the structural lipid that is cholesterol, and the PEG lipid is DMG-PEG.

In some embodiments, the lipid nanoparticle comprises 45 – 55 mole percent ionizable cationic lipid. For example, lipid nanoparticle may comprise 45, 46, 47, 48, 49, 50, 51, 52, 53, 54, or 55 mole percent ionizable cationic lipid.

In some embodiments, the lipid nanoparticle comprises 5 – 15 mole percent DSPC. For example, the lipid nanoparticle may comprise 5, 6, 7, 8, 9, 10, 11, 12, 13, 14, or 15 mole percent DSPC.

In some embodiments, the lipid nanoparticle comprises 35 – 40 mole percent cholesterol. For example, the lipid nanoparticle may comprise 35, 36, 37, 38, 39, or 40 mole percent cholesterol.

In some embodiments, the lipid nanoparticle comprises 1 – 2 mole percent DMG-PEG. For example, the lipid nanoparticle may comprise 1, 1.5, or 2 mole percent DMG-PEG.

In some embodiments, the lipid nanoparticle comprises 50 mole percent ionizable cationic lipid, 10 mole percent DSPC, 38.5 mole percent cholesterol, and 1.5 mole percent DMG-PEG.

In some embodiments, a LNP of the disclosure comprises an N:P ratio of from about 2:1 to about 30:1.

In some embodiments, a LNP of the disclosure comprises an N:P ratio of about 6:1.

In some embodiments, a LNP of the disclosure comprises an N:P ratio of about 3:1.

In some embodiments, a LNP of the disclosure comprises a wt/wt ratio of the ionizable cationic lipid component to the RNA of from about 10:1 to about 100:1.

In some embodiments, a LNP of the disclosure comprises a wt/wt ratio of the ionizable cationic lipid component to the RNA of about 20:1.

In some embodiments, a LNP of the disclosure comprises a wt/wt ratio of the ionizable cationic lipid component to the RNA of about 10:1.

In some embodiments, a LNP of the disclosure has a mean diameter from about 50 nm to about 150 nm.

In some embodiments, a LNP of the disclosure has a mean diameter from about 70 nm to about 120 nm.

### Multivalent Vaccines

The compositions, as provided herein, may include RNA or multiple RNAs encoding two or more antigens of the same or different species. In some embodiments, composition includes an RNA or multiple RNAs encoding two or more respiratory virus antigens. In some embodiments, the RNA may encode 1, 2, 3, 4, 5, 6, 7, 8, 9, 10, 11, 12, or more respiratory virus antigens.

In some embodiments, composition comprises an RNA encoding a hRSV F glycoprotein, an RNA encoding a hMPV F glycoprotein, and a hPIV3 F glycoprotein antigen.

In some embodiments, two or more different RNA (e.g., mRNA) encoding antigens may be formulated in the same lipid nanoparticle. In other embodiments, two or more different RNA encoding antigens may be formulated in separate lipid nanoparticles (each RNA formulated in a single lipid nanoparticle). The lipid nanoparticles may then be combined and administered as a single vaccine composition (e.g., comprising multiple RNA encoding multiple antigens) or may be administered separately.

### Combination Vaccines

The compositions, as provided herein, may include an RNA or multiple RNAs encoding two or more antigens of the same or different viral strains. Also provided herein are combination vaccines that include RNA encoding one or more hRSV antigen(s) and one or more antigen(s) of a different organism, such as hMPV and/or hPIV3. Thus, the vaccines of the present disclosure may be combination vaccines that target one or more antigens of the same strain/species, or one or more antigens of different strains/species, e.g., antigens which induce immunity to organisms which are found in the same geographic areas where the risk of respiratory virus infection is high or organisms to which an individual is likely to be exposed to when exposed to a respiratory virus.

### Pharmaceutical Formulations

Provided herein are compositions (e.g., pharmaceutical compositions), methods, kits and reagents for prevention or treatment of respiratory viruses in humans and other mammals, for example. The compositions provided herein can be used as therapeutic or prophylactic agents. They may be used in medicine to prevent and/or treat a respiratory virus infection.

In some embodiments, the respiratory virus vaccine containing RNA as described herein can be administered to a subject (e.g., a mammalian subject, such as a human subject), and the RNA polynucleotides are translated in vivo to produce an antigenic polypeptide (antigen).

An “effective amount” of a composition (e.g., comprising RNA) is based, at least in part, on the target tissue, target cell type, means of administration, physical characteristics of the RNA (e.g., length, nucleotide composition, and/or extent of modified nucleosides), other components of the vaccine, and other determinants, such as age, body weight, height, sex and general health of the subject. Typically, an effective amount of a composition provides an induced or boosted immune response as a function of antigen production in the cells of the subject. In some embodiments, an effective amount of the composition containing RNA polynucleotides having at least one chemical modifications are more efficient than a composition containing a corresponding unmodified polynucleotide encoding the same antigen or a peptide antigen. Increased antigen production may be demonstrated by increased cell transfection (the percentage of cells transfected with the RNA vaccine), increased protein translation and/or expression from the polynucleotide, decreased nucleic acid degradation (as demonstrated, for example, by increased duration of protein translation from a modified polynucleotide), or altered antigen specific immune response of the host cell.

The term “pharmaceutical composition” refers to the combination of an active agent with a carrier, inert or active, making the composition especially suitable for diagnostic or therapeutic use in vivo or ex vivo. A “pharmaceutically acceptable carrier,” after administered to or upon a subject, does not cause undesirable physiological effects. The carrier in the pharmaceutical composition must be “acceptable” also in the sense that it is compatible with the active ingredient and can be capable of stabilizing it. One or more solubilizing agents can be utilized as pharmaceutical carriers for delivery of an active agent. Examples of a pharmaceutically acceptable carrier include, but are not limited to, biocompatible vehicles, adjuvants, additives, and diluents to achieve a composition usable as a dosage form. Examples of other carriers include colloidal silicon oxide, magnesium stearate, cellulose, and sodium lauryl sulfate. Additional suitable pharmaceutical carriers and diluents, as well as pharmaceutical necessities for their use, are described in Remington’s Pharmaceutical Sciences.

In some embodiments, the compositions (comprising polynucleotides and their encoded polypeptides) in accordance with the present disclosure may be used for treatment or prevention of a respiratory virus infection. A composition may be administered prophylactically or therapeutically as part of an active immunization scheme to healthy individuals or early in infection during the incubation phase or during active infection after onset of symptoms. In some embodiments, the amount of RNA provided to a cell, a tissue or a subject may be an amount effective for immune prophylaxis.

A composition may be administered with other prophylactic or therapeutic compounds. As a non-limiting example, a prophylactic or therapeutic compound may be an adjuvant or a booster. As used herein, when referring to a prophylactic composition, such as a vaccine, the term “booster” refers to an extra administration of the prophylactic (vaccine) composition. A booster (or booster vaccine) may be given after an earlier administration of the prophylactic composition. The time of administration between the initial administration of the prophylactic composition and the booster may be, but is not limited to, 1 minute, 2 minutes, 3 minutes, 4 minutes, 5 minutes, 6 minutes, 7 minutes, 8 minutes, 9 minutes, 10 minutes, 15 minutes, 20 minutes 35 minutes, 40 minutes, 45 minutes, 50 minutes, 55 minutes, 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 1 day, 36 hours, 2 days, 3 days, 4 days, 5 days, 6 days, 1 week, 10 days, 2 weeks, 3 weeks, 1 month, 2 months, 3 months, 4 months, 5 months, 6 months, 7 months, 8 months, 9 months, 10 months, 11 months, 1 year, 18 months, 2 years, 3 years, 4 years, 5 years, 6 years, 7 years, 8 years, 9 years, 10 years, 11 years, 12 years, 13 years, 14 years, 15 years, 16 years, 17 years, 18 years, 19 years, 20 years, 25 years, 30 years, 35 years, 40 years, 45 years, 50 years, 55 years, 60 years, 65 years, 70 years, 75 years, 80 years, 85 years, 90 years, 95 years or more than 99 years. In exemplary embodiments, the time of administration between the initial administration of the prophylactic composition and the booster may be, but is not limited to, 1 week, 2 weeks, 3 weeks, 1 month, 2 months, 3 months, 6 months or 1 year.

In some embodiments, a composition may be administered intramuscularly, intranasally or intradermally, similarly to the administration of inactivated vaccines known in the art.

A composition may be utilized in various settings depending on the prevalence of the infection or the degree or level of unmet medical need. As a non-limiting example, the RNA vaccines may be utilized to treat and/or prevent a variety of infectious disease. RNA vaccines have superior properties in that they produce much larger antibody titers, better neutralizing immunity, produce more durable immune responses, and/or produce responses earlier than commercially available vaccines.

Provided herein are pharmaceutical compositions including RNA and/or complexes optionally in combination with one or more pharmaceutically acceptable excipients.

The RNA may be formulated or administered alone or in conjunction with one or more other components. For example, an immunizing composition may comprise other components including, but not limited to, adjuvants.

In some embodiments, an immunizing composition does not include an adjuvant (they are adjuvant free).

An RNA may be formulated or administered in combination with one or more pharmaceutically-acceptable excipients. In some embodiments, vaccine compositions comprise at least one additional active substances, such as, for example, a therapeutically-active substance, a prophylactically-active substance, or a combination of both. Vaccine compositions may be sterile, pyrogen-free or both sterile and pyrogen-free. General considerations in the formulation and/or manufacture of pharmaceutical agents, such as vaccine compositions, may be found, for example, in Remington: The Science and Practice of Pharmacy 21st ed., Lippincott Williams & Wilkins, 2005 (incorporated herein by reference in its entirety).

In some embodiments, an immunizing composition is administered to humans, human patients or subjects. For the purposes of the present disclosure, the phrase “active ingredient” generally refers to the RNA vaccines or the polynucleotides contained therein, for example, RNA polynucleotides (e.g., mRNA polynucleotides) encoding antigens.

Formulations of the vaccine compositions described herein may be prepared by any method known or hereafter developed in the art of pharmacology. In general, such preparatory methods include the step of bringing the active ingredient (e.g., mRNA polynucleotide) into association with an excipient and/or one or more other accessory ingredients, and then, if necessary and/or desirable, dividing, shaping and/or packaging the product into a desired singleor multi-dose unit.

Relative amounts of the active ingredient, the pharmaceutically acceptable excipient, and/or any additional ingredients in a pharmaceutical composition in accordance with the disclosure will vary, depending upon the identity, size, and/or condition of the subject treated and further depending upon the route by which the composition is to be administered. By way of example, the composition may comprise between 0.1% and 100%, e.g., between 0.5 and 50%, between 1-30%, between 5-80%, at least 80% (w/w) active ingredient.

In some embodiments, an RNA is formulated using one or more excipients to: (1) increase stability; (2) increase cell transfection; (3) permit the sustained or delayed release (e.g., from a depot formulation); (4) alter the biodistribution (e.g., target to specific tissues or cell types); (5) increase the translation of encoded protein in vivo; and/or (6) alter the release profile of encoded protein (antigen) in vivo. In addition to traditional excipients such as any and all solvents, dispersion media, diluents, or other liquid vehicles, dispersion or suspension aids, surface active agents, isotonic agents, thickening or emulsifying agents, preservatives, excipients can include, without limitation, lipidoids, liposomes, lipid nanoparticles, polymers, lipoplexes, core-shell nanoparticles, peptides, proteins, cells transfected with the RNA (e.g., for transplantation into a subject), hyaluronidase, nanoparticle mimics and combinations thereof.

### Dosing/Administration

Provided herein are immunizing compositions (e.g., RNA vaccines), methods, kits and reagents for prevention and/or treatment of respiratory virus infection in humans and other mammals. Immunizing compositions can be used as therapeutic or prophylactic agents. In some embodiments, immunizing compositions are used to provide prophylactic protection from respiratory virus infection. In some embodiments, immunizing compositions are used to treat a respiratory virus infection. In some embodiments, embodiments, immunizing compositions are used in the priming of immune effector cells, for example, to activate peripheral blood mononuclear cells (PBMCs) ex vivo, which are then infused (re-infused) into a subject.

A subject may be any mammal, including non-human primate and human subjects. Typically, a subject is a human subject.

In some embodiments, an immunizing composition (e.g., RNA a vaccine) is administered to a subject (e.g., a mammalian subject, such as a human subject) in an effective amount to induce an antigen-specific immune response. The RNA encoding the respiratory virus antigen is expressed and translated in vivo to produce the antigen, which then stimulates an immune response in the subject.

Prophylactic protection from a respiratory virus can be achieved following administration of an immunizing composition (e.g., an RNA vaccine) of the present disclosure. Immunizing compositions can be administered once, twice, three times, four times or more but it is likely sufficient to administer the vaccine once (optionally followed by a single booster). It is possible, although less desirable, to administer an immunizing compositions to an infected individual to achieve a therapeutic response. Dosing may need to be adjusted accordingly.

A method of eliciting an immune response in a subject against a respiratory virus antigen (or multiple antigens) is provided in aspects of the present disclosure. In some embodiments, a method involves administering to the subject an immunizing composition comprising a RNA (e.g., mRNA) having an open reading frame encoding a respiratory virus antigen (e.g., hRSV F glycoprotein, hMPV F glycoprotein, and/or hPIV3 F glycoprotein), thereby inducing in the subject an immune response specific to the respiratory virus antigen, wherein anti-antigen antibody titer in the subject is increased following vaccination relative to anti-antigen antibody titer in a subject vaccinated with a prophylactically effective dose of a traditional vaccine against the antigen. An “anti-antigen antibody” is a serum antibody the binds specifically to the antigen.

A prophylactically effective dose is an effective dose that prevents infection with the virus at a clinically acceptable level. In some embodiments, the effective dose is a dose listed in a package insert for the vaccine. A traditional vaccine, as used herein, refers to a vaccine other than the mRNA vaccines of the present disclosure. For instance, a traditional vaccine includes, but is not limited, to live microorganism vaccines, killed microorganism vaccines, subunit vaccines, protein antigen vaccines, DNA vaccines, virus like particle (VLP) vaccines, etc. In exemplary embodiments, a traditional vaccine is a vaccine that has achieved regulatory approval and/or is registered by a national drug regulatory body, for example the Food and Drug Administration (FDA) in the United States or the European Medicines Agency (EMA).

In some embodiments, the anti-antigen antibody titer in the subject is increased 1 log to 10 log following vaccination relative to anti-antigen antibody titer in a subject vaccinated with a prophylactically effective dose of a traditional vaccine against the respiratory virus or an unvaccinated subject. In some embodiments, the anti-antigen antibody titer in the subject is increased 1 log, 2 log, 3 log, 4 log, 5 log, or 10 log following vaccination relative to anti-antigen antibody titer in a subject vaccinated with a prophylactically effective dose of a traditional vaccine against the respiratory virus or an unvaccinated subject.

A method of eliciting an immune response in a subject against a respiratory virus is provided in other aspects of the disclosure. The method involves administering to the subject an immunizing composition (e.g., an RNA vaccine) comprising a RNA polynucleotide comprising an open reading frame encoding a respiratory virus antigen, thereby inducing in the subject an immune response specific to the respiratory virus, wherein the immune response in the subject is equivalent to an immune response in a subject vaccinated with a traditional vaccine against the respiratory virus at 2 times to 100 times the dosage level relative to the immunizing composition.

In some embodiments, the immune response in the subject is equivalent to an immune response in a subject vaccinated with a traditional vaccine at twice the dosage level relative to an immunizing composition of the present disclosure. In some embodiments, the immune response in the subject is equivalent to an immune response in a subject vaccinated with a traditional vaccine at three times the dosage level relative to an immunizing composition of the present disclosure. In some embodiments, the immune response in the subject is equivalent to an immune response in a subject vaccinated with a traditional vaccine at 4 times, 5 times, 10 times, 50 times, or 100 times the dosage level relative to an immunizing composition of the present disclosure. In some embodiments, the immune response in the subject is equivalent to an immune response in a subject vaccinated with a traditional vaccine at 10 times to 1000 times the dosage level relative to an immunizing composition of the present disclosure. In some embodiments, the immune response in the subject is equivalent to an immune response in a subject vaccinated with a traditional vaccine at 100 times to 1000 times the dosage level relative to an immunizing composition of the present disclosure.

In other embodiments, the immune response is assessed by determining [protein] antibody titer in the subject. In other embodiments, the ability of serum or antibody from an immunized subject is tested for its ability to neutralize viral uptake or reduce respiratory virus transformation of human B lymphocytes. In other embodiments, the ability to promote a robust T cell response(s) is measured using art recognized techniques.

Other aspects the disclosure provide methods of eliciting an immune response in a subject against a respiratory virus by administering to the subject an immunizing composition (e.g., an RNA vaccine) comprising an RNA having an open reading frame encoding a respiratory virus antigen, thereby inducing in the subject an immune response specific to the respiratory virus antigen, wherein the immune response in the subject is induced 2 days to 10 weeks earlier relative to an immune response induced in a subject vaccinated with a prophylactically effective dose of a traditional vaccine against the respiratory virus. In some embodiments, the immune response in the subject is induced in a subject vaccinated with a prophylactically effective dose of a traditional vaccine at 2 times to 100 times the dosage level relative to an immunizing composition of the present disclosure.

In some embodiments, the immune response in the subject is induced 2 days, 3 days, 1 week, 2 weeks, 3 weeks, 5 weeks, or 10 weeks earlier relative to an immune response induced in a subject vaccinated with a prophylactically effective dose of a traditional vaccine.

Also provided herein are methods of eliciting an immune response in a subject against a respiratory virus by administering to the subject an RNA having an open reading frame encoding a first antigen, wherein the RNA does not include a stabilization element, and wherein an adjuvant is not co-formulated or co-administered with the vaccine.

An immunizing composition (e.g., an RNA vaccine) may be administered by any route that results in a therapeutically effective outcome. These include, but are not limited, to intradermal, intramuscular, intranasal, and/or subcutaneous administration. The present disclosure provides methods comprising administering RNA vaccines to a subject in need thereof. The exact amount required will vary from subject to subject, depending on the species, age, and general condition of the subject, the severity of the disease, the particular composition, its mode of administration, its mode of activity, and the like. The RNA is typically formulated in dosage unit form for ease of administration and uniformity of dosage. It will be understood, however, that the total daily usage of the RNA may be decided by the attending physician within the scope of sound medical judgment. The specific therapeutically effective, prophylactically effective, or appropriate imaging dose level for any particular patient will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts.

The effective amount of the RNA, as provided herein, may be as low as 20 µg, administered for example as a single dose or as two 10 µg doses. In some embodiments, the effective amount is a total dose of 20 µg-300 µg or 25 µg-300 µg. For example, the effective amount may be a total dose of 20 µg, 25 µg, 30 µg, 35 µg, 40 µg, 45 µg, 50 µg, 55 µg, 60 µg, 65 µg, 70 µg, 75 µg, 80 µg, 85 µg, 90 µg, 95 µg, 100 µg, 110 µg, 120 µg, 130 µg, 140 µg, 150 µg, 160 µg, 170 µg, 180 µg, 190 µg, 200 µg, 250 µg, or 300 µg. In some embodiments, the effective amount is a total dose of 25 µg-300 µg. In some embodiments, the effective amount is a total dose of 20 µg. In some embodiments, the effective amount is a total dose of 25 µg. In some embodiments, the effective amount is a total dose of 75 µg. In some embodiments, the effective amount is a total dose of 150 µg. In some embodiments, the effective amount is a total dose of 300 µg.

The RNA described herein can be formulated into a dosage form described herein, such as an intranasal, intratracheal, or injectable (e.g., intravenous, intraocular, intravitreal, intramuscular, intradermal, intracardiac, intraperitoneal, and subcutaneous).

### Vaccine Efficacy

Some aspects of the present disclosure provide formulations of the immunizing compositions (e.g., RNA vaccines), wherein the RNA is formulated in an effective amount to produce an antigen specific immune response in a subject (e.g., production of antibodies specific to a respiratory virus antigen). “An effective amount” is a dose of the RNA effective to produce an antigen-specific immune response. Also provided herein are methods of inducing an antigen-specific immune response in a subject.

As used herein, an immune response to a vaccine or LNP of the present disclosure is the development in a subject of a humoral and/or a cellular immune response to a (one or more) respiratory virus protein(s) present in the vaccine. For purposes of the present disclosure, a “humoral” immune response refers to an immune response mediated by antibody molecules, including, e.g., secretory (IgA) or IgG molecules, while a “cellular” immune response is one mediated by T-lymphocytes (e.g., CD4+ helper and/or CD8+ T cells (e.g., CTLs) and/or other white blood cells. One important aspect of cellular immunity involves an antigen-specific response by cytolytic T-cells (CTLs). CTLs have specificity for peptide antigens that are presented in association with proteins encoded by the major histocompatibility complex (MHC) and expressed on the surfaces of cells. CTLs help induce and promote the destruction of intracellular microbes or the lysis of cells infected with such microbes. Another aspect of cellular immunity involves and antigen-specific response by helper T-cells. Helper T-cells act to help stimulate the function, and focus the activity nonspecific effector cells against cells displaying peptide antigens in association with MHC molecules on their surface. A cellular immune response also leads to the production of cytokines, chemokines, and other such molecules produced by activated T-cells and/or other white blood cells including those derived from CD4+ and CD8+ T-cells.

In some embodiments, the antigen-specific immune response is characterized by measuring an anti-respiratory virus antigen antibody titer produced in a subject administered an immunizing composition as provided herein. An antibody titer is a measurement of the amount of antibodies within a subject, for example, antibodies that are specific to a particular antigen (e.g., an anti-hRSV F glycoprotein) or epitope of an antigen. Antibody titer is typically expressed as the inverse of the greatest dilution that provides a positive result. Enzyme-linked immunosorbent assay (ELISA) is a common assay for determining antibody titers, for example.

In some embodiments, an antibody titer is used to assess whether a subject has had an infection or to determine whether immunizations are required. In some embodiments, an antibody titer is used to determine the strength of an autoimmune response, to determine whether a booster immunization is needed, to determine whether a previous vaccine was effective, and to identify any recent or prior infections. In accordance with the present disclosure, an antibody titer may be used to determine the strength of an immune response induced in a subject by an immunizing composition (e.g., RNA vaccine).

In some embodiments, an anti-respiratory virus antigen antibody titer produced in a subject is increased by at least 1 log relative to a control. For example, anti-respiratory virus antigen antibody titer produced in a subject may be increased by at least 1.5, at least 2, at least 2.5, or at least 3 log relative to a control. In some embodiments, the anti-respiratory virus antigen antibody titer produced in the subject is increased by 1, 1.5, 2, 2.5 or 3 log relative to a control. In some embodiments, the anti-respiratory virus antigen antibody titer produced in the subject is increased by 1-3 log relative to a control. For example, the anti-respiratory virus antigen antibody titer produced in a subject may be increased by 1-1.5, 1-2, 1-2.5, 1-3, 1.5-2, 1.5-2.5, 1.5-3, 2-2.5, 2-3, or 2.5-3 log relative to a control.

In some embodiments, the anti-respiratory virus antigen antibody titer produced in a subject is increased at least 2 times relative to a control. For example, the anti-respiratory virus antigen n antibody titer produced in a subject may be increased at least 3 times, at least 4 times, at least 5 times, at least 6 times, at least 7 times, at least 8 times, at least 9 times, or at least 10 times relative to a control. In some embodiments, the anti-respiratory virus antigen antibody titer produced in the subject is increased 2, 3, 4, 5, 6, 7, 8, 9, or 10 times relative to a control. In some embodiments, the anti-respiratory virus antigen antibody titer produced in a subject is increased 2-10 times relative to a control. For example, the anti-respiratory virus antigen antibody titer produced in a subject may be increased 2-10, 2-9, 2-8, 2-7, 2-6, 2-5, 2-4, 2-3, 3-10, 3-9, 3-8, 3-7, 3-6, 3-5, 3-4, 4-10, 4-9, 4-8, 4-7, 4-6, 4-5, 5-10, 5-9, 5-8, 5-7, 5-6, 6-10, 6-9, 6-8, 6-7, 7-10, 7-9, 7-8, 8-10, 8-9, or 9-10 times relative to a control.

In some embodiments, an antigen-specific immune response is measured as a ratio of geometric mean titer (GMT), referred to as a geometric mean ratio (GMR), of serum neutralizing antibody titers to hRSV, hMPV, and/or hPIV3. A geometric mean titer (GMT) is the average antibody titer for a group of subjects calculated by multiplying all values and taking the nth root of the number, where n is the number of subjects with available data.

A control, in some embodiments, is an anti-respiratory virus antigen antibody titer produced in a subject who has not been administered an immunizing composition (e.g., RNA vaccine). In some embodiments, a control is an anti-respiratory virus antigen antibody titer produced in a subject administered a recombinant or purified protein vaccine. Recombinant protein vaccines typically include protein antigens that either have been produced in a heterologous expression system (e.g., bacteria or yeast) or purified from large amounts of the pathogenic organism.

In some embodiments, the ability of an immunizing composition (e.g., RNA vaccine) to be effective is measured in a murine model. For example, an immunizing composition may be administered to a murine model and the murine model assayed for induction of neutralizing antibody titers. Viral challenge studies may also be used to assess the efficacy of a vaccine of the present disclosure. For example, an immunizing composition may be administered to a murine model, the murine model challenged with virus, and the murine model assayed for survival and/or immune response (e.g., neutralizing antibody response, T cell response (e.g., cytokine response)).

In some embodiments, an effective amount of an immunizing composition (e.g., RNA vaccine) is a dose that is reduced compared to the standard of care dose of a recombinant protein vaccine. A “standard of care,” as provided herein, refers to a medical or psychological treatment guideline and can be general or specific. “Standard of care” specifies appropriate treatment based on scientific evidence and collaboration between medical professionals involved in the treatment of a given condition. It is the diagnostic and treatment process that a physician/ clinician should follow for a certain type of patient, illness or clinical circumstance. A “standard of care dose,” as provided herein, refers to the dose of a recombinant or purified protein vaccine, or a live attenuated or inactivated vaccine, or a VLP vaccine, that a physician/clinician or other medical professional would administer to a subject to treat or prevent respiratory virus infection or a related condition, while following the standard of care guideline for treating or preventing respiratory virus infection or a related condition.

In some embodiments, the anti-respiratory virus antigen antibody titer produced in a subject administered an effective amount of an immunizing composition is equivalent to an anti-respiratory virus antigen antibody titer produced in a control subject administered a standard of care dose of a recombinant or purified protein vaccine, or a live attenuated or inactivated vaccine, or a VLP vaccine.

Vaccine efficacy may be assessed using standard analyses (see, e.g., Weinberg et al., J Infect Dis. 2010 Jun 1;201(11):1607-10). For example, vaccine efficacy may be measured by double-blind, randomized, clinical controlled trials. Vaccine efficacy may be expressed as a proportionate reduction in disease attack rate (AR) between the unvaccinated (ARU) and vaccinated (ARV) study cohorts and can be calculated from the relative risk (RR) of disease among the vaccinated group with use of the following formulas:

$$\text{Efficacy =} \left(\text{ARU} - \text{ARV}\right) / \text{ARU x 100; and}$$

$$\text{Efficacy =} \left(1 \text{-RR}\right) \text{x 100} \text{.}$$

Likewise, vaccine effectiveness may be assessed using standard analyses (see, e.g., Weinberg et al., J Infect Dis. 2010 Jun 1;201(11):1607-10). Vaccine effectiveness is an assessment of how a vaccine (which may have already proven to have high vaccine efficacy) reduces disease in a population. This measure can assess the net balance of benefits and adverse effects of a vaccination program, not just the vaccine itself, under natural field conditions rather than in a controlled clinical trial. Vaccine effectiveness is proportional to vaccine efficacy (potency) but is also affected by how well target groups in the population are immunized, as well as by other non-vaccine-related factors that influence the ‘real-world’ outcomes of hospitalizations, ambulatory visits, or costs. For example, a retrospective case control analysis may be used, in which the rates of vaccination among a set of infected cases and appropriate controls are compared. Vaccine effectiveness may be expressed as a rate difference, with use of the odds ratio (OR) for developing infection despite vaccination:

$$\text{Effectiveness =} \left(1 - \text{OR}\right) \text{x 100} \text{.}$$

In some embodiments, efficacy of the immunizing composition (e.g., RNA vaccine) is at least 60% relative to unvaccinated control subjects. For example, efficacy of the immunizing composition may be at least 65%, at least 70%, at least 75%, at least 80%, at least 85%, at least 95%, at least 98%, or 100% relative to unvaccinated control subjects.

Sterilizing Immunity. Sterilizing immunity refers to a unique immune status that prevents effective pathogen infection into the host. In some embodiments, the effective amount of an immunizing composition of the present disclosure is sufficient to provide sterilizing immunity in the subject for at least 1 year. For example, the effective amount of an immunizing composition of the present disclosure is sufficient to provide sterilizing immunity in the subject for at least 2 years, at least 3 years, at least 4 years, or at least 5 years. In some embodiments, the effective amount of an immunizing composition of the present disclosure is sufficient to provide sterilizing immunity in the subject at an at least 5-fold lower dose relative to control. For example, the effective amount may be sufficient to provide sterilizing immunity in the subject at an at least 10-fold lower, 15-fold, or 20-fold lower dose relative to a control.

Detectable Antigen. In some embodiments, the effective amount of an immunizing composition of the present disclosure is sufficient to produce detectable levels of respiratory virus antigen as measured in serum of the subject at 1-72 hours post administration.

Titer. An antibody titer is a measurement of the amount of antibodies within a subject, for example, antibodies that are specific to a particular antigen (e.g., an anti-respiratory virus antigen). Antibody titer is typically expressed as the inverse of the greatest dilution that provides a positive result. Enzyme-linked immunosorbent assay (ELISA) is a common assay for determining antibody titers, for example.

In some embodiments, the effective amount of an immunizing composition of the present disclosure is sufficient to produce a 1,000-10,000 neutralizing antibody titer produced by neutralizing antibody against the respiratory virus antigen as measured in serum of the subject at 1-72 hours post administration. In some embodiments, the effective amount is sufficient to produce a 1,000-5,000 neutralizing antibody titer produced by neutralizing antibody against the respiratory virus antigen as measured in serum of the subject at 1-72 hours post administration. In some embodiments, the effective amount is sufficient to produce a 5,000-10,000 neutralizing antibody titer produced by neutralizing antibody against the respiratory virus antigen as measured in serum of the subject at 1-72 hours post administration.

In some embodiments, the neutralizing antibody titer is at least 100 NT₅₀. For example, the neutralizing antibody titer may be at least 200, 300, 400, 500, 600, 700, 800, 900 or 1000 NT₅₀. In some embodiments, the neutralizing antibody titer is at least 10,000 NT₅₀.

In some embodiments, the neutralizing antibody titer is at least 100 neutralizing units per milliliter (NU/mL). For example, the neutralizing antibody titer may be at least 200, 300, 400, 500, 600, 700, 800, 900 or 1000 NU/mL. In some embodiments, the neutralizing antibody titer is at least 10,000 NU/mL.

In some embodiments, an anti-respiratory virus antigen antibody titer produced in the subject is increased by at least 1 log relative to a control. For example, an anti-respiratory virus antigen antibody titer produced in the subject may be increased by at least 2, 3, 4, 5, 6, 7, 8, 9 or 10 log relative to a control.

In some embodiments, an anti-respiratory virus antigen antibody titer produced in the subject is increased at least 2 times relative to a control. For example, an anti-respiratory virus antigen antibody titer produced in the subject is increased by at least 3, 4, 5, 6, 7, 8, 9 or 10 times relative to a control.

In some embodiments, a geometric mean, which is the nth root of the product of n numbers, is generally used to describe proportional growth. Geometric mean, in some embodiments, is used to characterize antibody titer produced in a subject.

A control may be, for example, an unvaccinated subject, or a subject administered a live attenuated viral vaccine, an inactivated viral vaccine, or a protein subunit vaccine.

### EXAMPLES

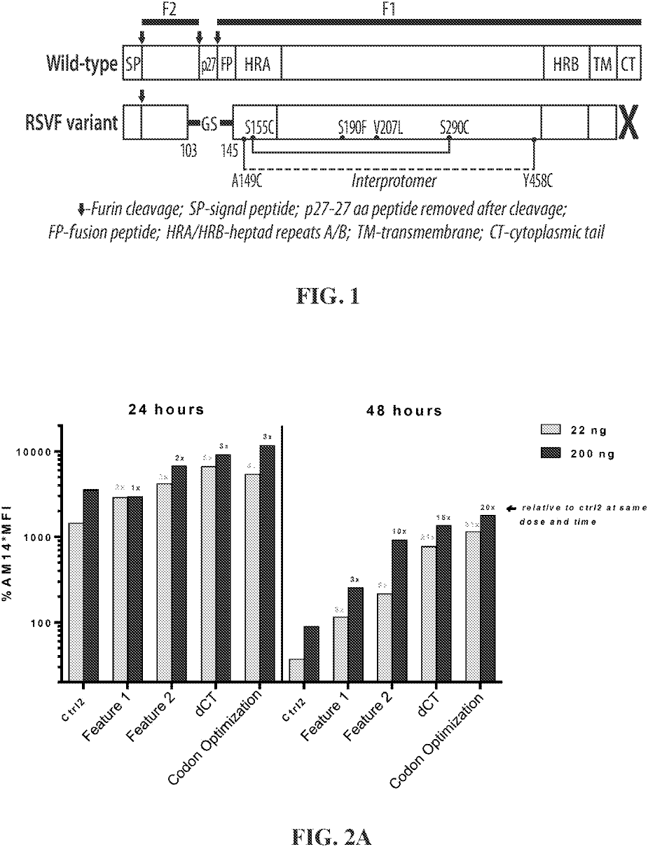

The effects of different features (e.g., modifications) to a wild-type hRSV F glycoprotein were examined. FIG. 1 is a schematic illustrating the differences between the wild-type mRNA encoding the F protein and the RSV F variant described herein. The RSV F variant is a codon-optimized, membrane-anchored, single chain mRNA that comprises interprotomer disulfide stabilizing mutations and cavity-filing mutations, in addition to lacking a cytoplasmic tail.

### Example 1 – mRNA Screening: Independent Features

As an increase in the prefusion form of RSV F glycoprotein on cells has been found to increase immunogenicity in animals. In this example, a variety of mRNAs were designed to test different features (e.g., different codon optimization strategies, mutations, specific modifications, structural changes) and their effect on resulting expression levels.

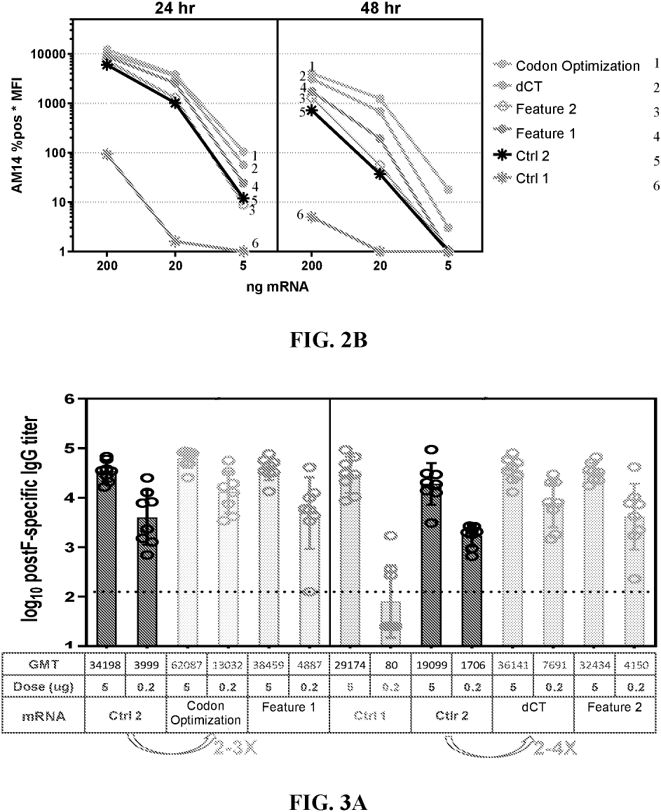

HEK293T cells were transfected with varying concentrations of the different mRNAs. Cell surface prefusion RSV F glycoprotein was detected 24 and 48 hours later by flow cytometry using an antibody specific to prefusion RSV F glycoprotein (AM14). The results, presented in FIG. 2A, demonstrate that two of the features tested, codon optimization and cytoplasmic tail truncation (“dCT”), generated the greatest increase in cell surface prefusion RSV F glycoprotein. For example, in the 48 hour group, the two features demonstrated an increase of 15-31 fold over that of the control (an mRNA encoding an RSV F glycoprotein that does not include the two features) at concentrations of 20 ng. This result was also seen at concentrations as low as 5 ng (FIG. 2B).

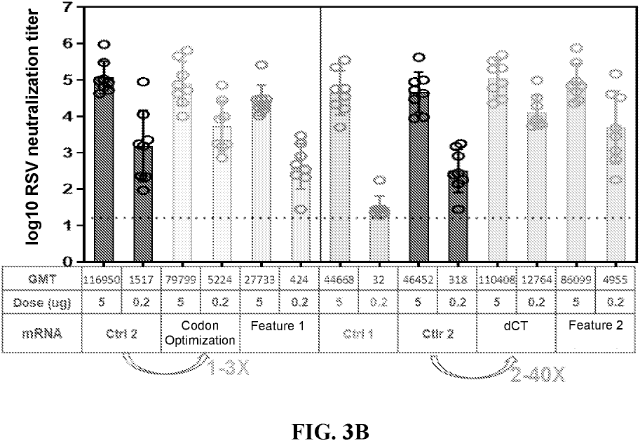

The codon-optimized mRNA and the mRNA encoding an RSV F glycoprotein having a cytoplasmic tail truncation were then screened in vivo. Eight-week-old BALB/c mice (n=8 per group) were dosed intramuscularly (IM) with the candidate mRNAs formulated in lipid nanoparticles (e.g., 0.5-15% PEG-modified lipid; 5-25% non-cationic lipid; 25-55% sterol; and 20-60% ionizable cationic lipid). The mRNAs were administered at a 3 week interval, and sera were collected after each dosing. Serum antibody titers against the F glycoprotein were determined with an ELISA. Following two doses (at post-dose 2, “PD2”), the postfusion F-specific IgG titer was measured. The codon-optimized mRNA and mRNA encoding an RSV F glycoprotein having a truncated cytoplasmic tail showed titers 2-3 fold higher and 2-4 fold higher those of the control (an mRNA encoding RSV F glycoprotein that does not include the two features), respectively (FIG. 3A).

The RSV neutralization titer was also measured after the second dose (PD2) using a microneutralization assay. Individual mouse sera were evaluated for neutralization of RSV-A (Long strain) using the following procedures:

1. All sera samples were heat inactivated by placing in dry bath incubator set at 56° C. for 30 minutes. Samples and control sera were then diluted 1:3 in virus diluent (2% FBS in EMEM) and duplicate samples were added to an assay plate and serially diluted.

2. RSV-Long stock virus was removed from the freezer and quickly thawed in 37° C. water bath. Viruses were diluted to 2000 pfu/mL in virus diluent

3. Diluted virus was added to each well of the 96-well plate, with the exception of one column of cells.

4. HEp-2 cells were trypsinized, washed, resuspended at 1.5 × 105 cells/ml in virus diluent, and 100 mL of the suspended cells were added to each well of the 96-well plate. The plates were then incubated for 72 hours at 37° C., 5% CO2.

5. Following the 72 hour incubation, the cells were washed with PBS, and fixed using 80% acetone dissolved in PBS for 10-20 minutes at 16-24° C. The fixative was removed and the plates were allowed to air-dry.

6. Plates were then washed thoroughly with PBS + 0.05% Tween. The detections monoclonal antibodies, 143-F3-1B8 and 34C9 were diluted to 2.5 plates were then washed thoroughly with PBS + 0.05% 50 plates were then washed thoroughly with PBS + 0.well of the 96-well plate. The plates were then incubated in a humid chamber at 16-24oC for 60-75 minutes on rocker

7. Following the incubation, the plates were thoroughly washed.

8. Biotinylated horse anti-mouse IgG was diluted 1:200 in assay diluent and added to each well of the 96-well plate. Plates were incubated as above and washed.

9. A cocktail of IRDye 800 CW Streptavidin (1:1000 final dilution), Sapphire 700 (1:1000 dilution) and 5 mM DRAQ5 solution (1:10,000 dilution) was prepared in assay diluent and 50 mL of the cocktail was added to each well of the 96-well plate. Plates were incubated as above in the dark, washed, and allowed to air dry.

10. Plates were then read using an Aerius Imager. Serum neutralizing titers were then calculated using a 4 parameter curve fit in Graphpad Prism.

The serum neutralizing antibody titers for the mouse immunogenicity study measured post dose 2 (PD2) are shown in FIG. 3B. The codon-optimized mRNA and the mRNA encoding an RSV F glycoprotein having a cytoplasmic tail truncation had 1-3 times and 2-40 times the titer levels of the control, indicating that the neutralizing antibody titers are robust. Therefore, it was found, both in vitro and in vivo, that the two mRNAs were able to increase expression of RSV F glycoprotein and RSV-A neutralization titer.

### Example 2 – mRNA Screening: Two Features

As the both codon-optimization and cytoplasmic tail truncation were shown to improve the expression and resulting immunogenicity of the RSV F protein, the combination of both features in the same mRNA was tested (“RSV F variant”). In an in vitro experiment, HEK293T cells were transfected with 20 ng or 200 ng of mRNAs having different combinations of the tested features. MRNAs comprising each feature individually were also screened. Cell surface prefusion RSV F glycoprotein was detected 24 and 48 hours later by flow cytometry using an antibody specific to prefusion RSV F glycoprotein (AM14). The combination of codon-optimization and cytoplasmic tail truncation was found to result in RSV F glycoprotein expression levels 5-50-fold higher relative to a control (an mRNA encoding an RSV F glycoprotein that does not include the two features). None of the other features, alone or paired in combination with one another, generated RSV F glycoproteins to the level that the selected combination did (data not shown).

The control RSV F-encoded proteins used were as follows: Ctrl1 contains 4 amino acid mutations in the F1 region relative to wild-type RSV F glycoprotein (FIG. 1), and does not contain the deletion between amino acids 103 and 145. As a result, Ctrl1 includes the wild-type furin cleavage site and retains the cytoplasmic domain. Another control variant, Ctrl2 is derived from the RSV F variant shown in FIG. 1, but does not include the C terminal deletion nor the other RNA optimizations and enhancements.

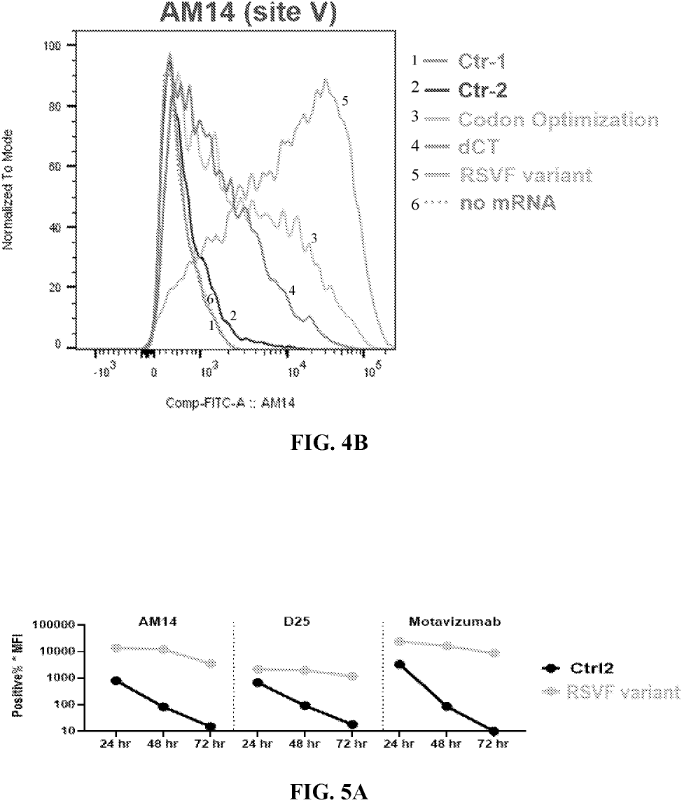

The RSV F variant was then screened further. In vitro expression of RSV F glycoprotein was measured in HEK293T cells transfected with 500 ng of the mRNA RSV F variant, a control mRNA, or no mRNA (negative control). Then, 24, 48, and 72 hours later, levels of RSV F glycoprotein were measured with flow cytometry. Three different antibodies were used to measure RSV F glycoprotein: AM14 and D25 (antibodies specific to the prefusion form of RSV F glycoprotein) and SYNAGIS@/motavizumab (directed to an epitope common to pre- and postfusion forms of RSV F glycoprotein). FIG. 4A demonstrates that the RSV F variant resulted in RSV F glycoprotein that was correctly folded in the prefusion conformation, and also in a higher and longer expression level as compared to the controls. Furthermore, FIGS. 5A and 5B demonstrate that the expression trends are not limited to HEK293T cells, and are maintained when performed in THP-1 cells (a human monocyte line).

In a further experiment, the in vitro expression of RSV F glycoprotein in HEK293T cells 48 hours after transfection with 200 ng of mRNA (RSV F variant, mRNA encoding RSV F glycoprotein having truncated cytoplasmic tail, codon-optimized mRNA encoding an RSV F glycoprotein, Ctrl1 and Ctrl2 mRNAs, or no mRNA) as determined by flow cytometry was compared. The RSV F variant showed expression levels that were at least additive, relative to a codon-optimized mRNA and an mRNA encoding an RSV F protein having a cytoplasmic tail truncation (FIG. 4B).

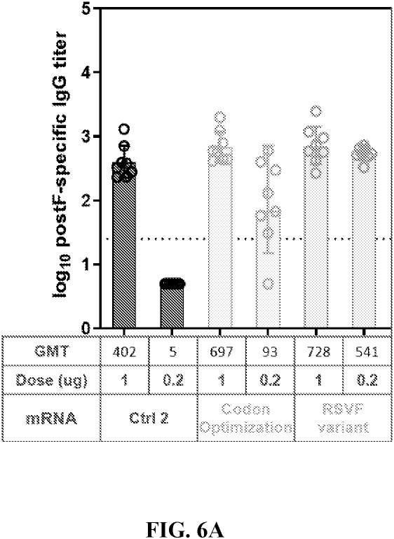

In vitro expression of RSV F glycoprotein in human peripheral blood mononuclear cells (huPBMCs) was examined. HuPBMCs were plated in a 12-well plate at a concentration of 1 × 10⁶ cells/well. 1000 ng of mRNA (either the RSV F variant or mRNAs encoding RSV F glycoprotein that do not include the two features) were then added to the wells, and the plates were incubated for 24 or 48 hours. Following incubation, the cells were stained with AM14-FITC (targeting the prefusion form of RSV F protein), D25-PE (targeting the prefusion form of RSV F protein), or motazuvimab-APC (targeting an epitope common to the pre- and post-fusion forms of RSV F protein). Higher levels of RSV F protein were observed with respect to the control mRNA and the cells that were not transfected, both at the 24 hour time point and at the 48 hour time point, regardless of the antibody used (FIG. 7).

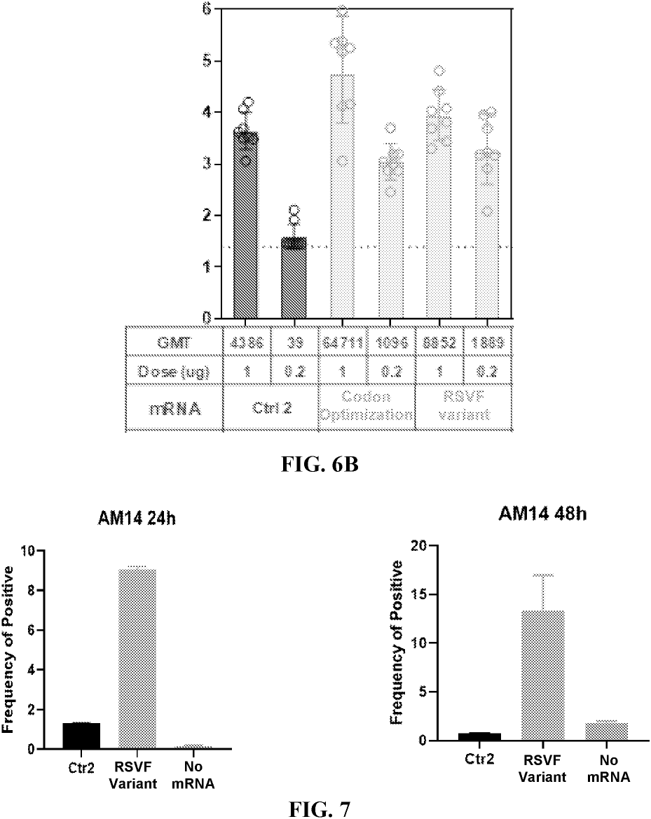

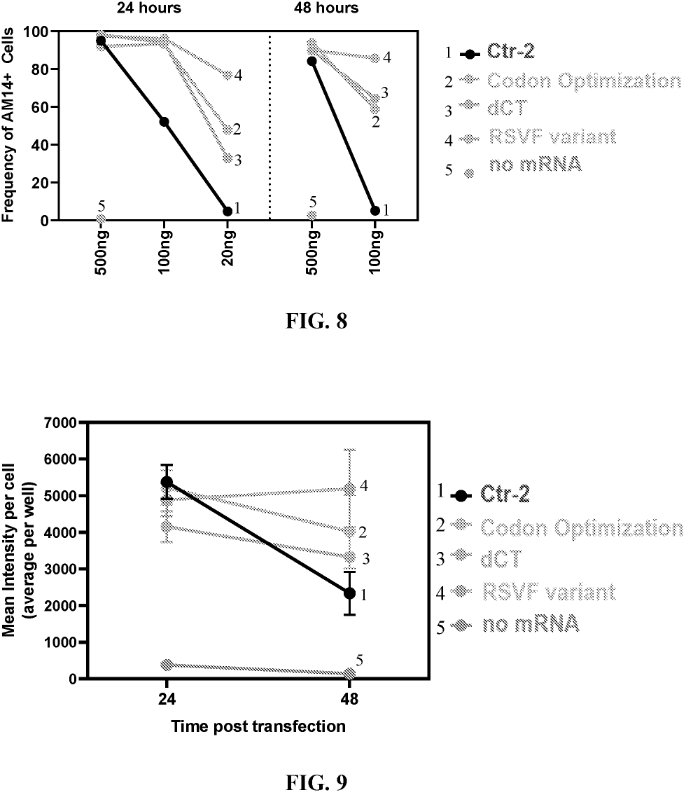

In vitro expression of the variant RSV F mRNA in human hepatoma HeP3B (HeP3B) cells was examined. HeP3B cells were plated in a 24 well plate and transfected with either 500 ng, 100 ng, or 20 ng of mRNAs. The plates were incubated for 24 or 48 hours. Following incubation, the cells were stained with AM14-FITC, targeting the prefusion form of the RSV F protein. Higher levels of RSV F protein were observed after incubation with the RSV F variant (codon optimization and cytoplasmic tail truncation) with respect to mRNAs having each individual feature (e.g., the codon-optimized mRNA and the mRNA encoding RSV F glycoprotein with a truncated cytoplasmic tail). The control mRNA (an mRNA encoding an RSV F glycoprotein that does not include the two features), and the cells that were not transfected (“no mRNA”) showed lower expression levels than the RSV F variant, in particular at 48 hours and at the lowest dose tested (FIG. 8). In microscopy experiments, it was found that the expression trends were consistent in HeLa cells. HeLa cells were plated in a 96 well plate and then transfected with 200 ng of mRNA (RSV F variant, a codon-optimized mRNA encoding an RSV F glycoprotein, an mRNA encoding an RSV F glycoprotein having a truncated cytoplasmic tail, a control mRNA that does not include the two features) or no mRNA (as a negative control)). The plates were incubated for 24 or 48 hours and then fixed in 4% PFA/PBS for 15 minutes and washed twice in PBS. Half the plate was then permeabilized in 0.5% Triton-X for 5 minutes, and then washed twice in PBS. The cells were then blocked in 1% BSA/PBS for 30 minutes at room temperature. Then, the primary antibody, an anti-RSV antibody (D25, Cambridge Bio) (diluted 1:100 in 1% BSA/PBS), was applied for one hour, followed by two washes in PBS. Then, the plates were blocked for 1% BSA for 10 minutes. A secondary antibody was applied for 30 minutes (diluted 1:2000 in BSA/PBS), and then the plates were washed twice with PBS. Then, NucBlue Fixed and CellMask Red were applied for 30 minutes, followed by washing twice with PBS. The resulting plates were then measured for protein expression using ALEXA488™. Mean fluorescent intensity was measured per cell based on cytoplasmic segmentation. As shown in FIG. 9, the RSV F variant (codon-optimized mRNA encoding a RSV F glycoprotein with cytoplasmic tail truncation) yielded the highest levels of RSV F protein. The difference between the control and RSV F variant was found to be about two-fold.

### Example 3 – In Vivo Immunogenicity Studies (Mice)

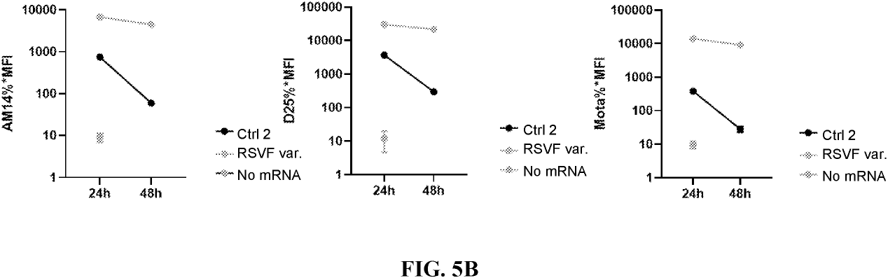

The RSV F variant (codon-optimized mRNA encoding a RSV F glycoprotein with cytoplasmic tail truncation) was then evaluated in vivo. Eight-week-old BALB/c mice (n=8 per group) were immunized intramuscularly (IM) with the RSV F variant or a control mRNA formulated in lipid nanoparticles (e.g., 0.5-15% PEG-modified lipid; 5-25% non-cationic lipid; 25-55% sterol; and 20-60% ionizable cationic lipid). The mRNAs were administered at a 3 week interval, and sera were collected after each immunization. Serum antibody titers against F glycoprotein were determined with an ELISA. Following the first and second doses, the postfusion F-specific IgG titer was measured. The RSV F variant had a titer at least 3-5 times that of the control (an alternative mRNA encoding an RSV F glycoprotein) at the low dose (200 ng) (FIGS. 6A and 6B).

An HRSV-A Virospot assay was performed to detect HRSV-specific neutralization antibodies in the sera samples. Briefly, samples were inactivated by incubating for 30 minutes at 56° C. Subsequently, serial two-fold dilutions of the samples were made in infection medium in triplicate in 96-wells plates starting with a dilution of 1:8 (first serum dilution in the test of 1:16). The sample dilutions were then incubated with a fixed amount of HRSV-A for 1 hour at 37° C. Then, the virus-antibody mixtures were transferred to plates with HEp-2 cell culture monolayers. After an incubation period of 1 day at 37° C., the monolayers were fixed and stained. The culture supernatants were removed, the monolayers were washed once with PBS, and then fixed with 50%/50% methanol/ethanol. After fixation, the plates were stained using a mouse monoclonal antibody against HRSV-A, a secondary HRP-labelled anti-mouse antibody, and TrueBlue. Stained plates were scanned using the IMMUNOSPOT® analyzer and 50% plaque reduction titers were calculated with the formula described by Zielinska et al. (Zielinska, Virology Journal 2005; 2(84): 1-5):

$$\text{X} \text{=} \left(\text{a-b}\right) \left(\text{e-c}\right) / \left(\text{c-d}\right) + \text{a}$$

where: X = neutralization result
a = log10 of dilution above the 50% reduction point
b = log 10 of dilution below the 50% reduction point
c = average SC above the 50% reduction point (corresponds with a)
d = average SC below the 50% reduction point (corresponds with b)
e = value of 50% reduction of average virus control count.

### Example 4 – In Vivo Immunogenicity Studies (Rats)

The RSV F variant (codon-optimized mRNA encoding a RSV F glycoprotein with cytoplasmic tail truncation) was then evaluated in vivo in cotton rats. The studies aimed to evaluate the immunogenicity, efficacy, and safety of the mRNA vaccine in the respiratory syncytial virus (RSV) cotton rat model, and include an evaluation of the potential for vaccineenhanced respiratory disease (ERD) over a range of dose levels, including those inducing suboptimal neutralizing antibody titers permitting detectable virus replication after challenge.

The RSV F variant or a control mRNA were formulated in lipid nanoparticles (e.g., 0.5-15% PEG-modified lipid; 5-25% non-cationic lipid; 25-55% sterol; and 20-60% ionizable cationic lipid). The components of the lipid nanoparticle comprised heptadecan-9-yl 8-((2-hydroxyethyl)(6-oxo-6(undecyloxy)hexyl) amino) octanoate (Compound 1); 1,2-dimyristoyl-racn-glycerol, methoxypolyethyleneglycol (PEG2000-DMG); 1,2-distearoyl-sn-glycero-3-phosphocholine (DSPC); and cholesterol.

Female cotton rats (6-8 weeks of age) were dived into 14 groups of 10 animals and a control group of four animals. The rats were immunized according to the schedule shown in Table 2 below. Groups 1-13 were immunized intramuscularly with 100 µL dose of the mRNA-LNP composition per animal; Group 14 was infected intranasally with 100 µL dose RSV /A2 at 10⁵ plaque forming units (PFUs) per animal. Some groups were immunized twice (Days 0 and 28), while others were immunized only on Day 0, as shown in Table 2. On Day 56, the mice were challenged with an intranasal administration of 0.1 mL of 5.0 log₁₀ RSV/A2. On day 61, the animals were sacrificed, the nasal tissue was harvested for viral titration measurements and the lungs were harvested en bloc and trisected; the left section for viral titrations, the lingular lobe for quantitative polymerase chain reaction (qPCR) analysis, and the right section was inflated and used for histopathology for enhanced RSV disease (ERD) and eosinophilia.

TABLE 2

Outline of Cotton Rat Study

| Group # | N | Treatment | Route | Dose Schedule | Challenge |
|---|---|---|---|---|---|
| 1 | 10 | 30 µg RSV mRNA-LNP | IM | Day 0, 28 | 5.0Log₁₀ PFU RSV |
| 2 | 10 | 3 µg RSV mRNA-LNP | IM | Day 0, 28 | 5.0Log₁₀ PFU RSV |
| 3 | 10 | 0.3 µg RSV mRNA-LNP | IM | Day 0, 28 | 5.0Log₁₀ PFU RSV |
| 4 | 10 | 0.03 µg RSV mRNA-LNP | IM | Day 0, 28 | 5.0Log₁₀ PFU RSV |
| 5 | 10 | 0.003 µg RSV mRNA-LNP | IM | Day 0, 28 | 5.0Log₁₀ PFU RSV |
| 6 | 10 | 0.0003 µg RSV mRNA-LNP | IM | Day 0, 28 | 5.0Log₁₀ PFU RSV |
| 7 | 10 | 0.3 µg RSV mRNA-LNP | IM | Day 0 | 5.0Log₁₀ PFU RSV |
| 8 | 10 | 0.03 µg RSV mRNA-LNP | IM | Day 0 | 5.0Log₁₀ PFU RSV |
| 9 | 10 | 1:100 formalin-inactivated RSV (FI-RSV) | IM | Day 0, 28 | 5.0Log₁₀ PFU RSV |
| 10 | 10 | 1:125 FI-RSV | IM | Day 0, 28 | 5.0Log₁₀ PFU RSV |
| 11 | 10 | 1:125 FI-mock | IM | Day 0, 28 | 5.0Log₁₀ PFU RSV |
| 12 | 10 | 30 µg mRNA/LNP control | IM | Day 0, 28 | 5.0Log₁₀ PFU RSV |
| 13 | 10 | PBS | IM | Day 0, 28 | 5.0Log₁₀ PFU RSV |
| 14 | 10 | 5.0Log₁₀ PFU RSV | IN | Day 0 | 5.0Log₁₀ PFU RSV |
| 15 | 4 | - | - | - | - |

For analysis, RSV/A2 lung and nose viral titrations were performed. Lung and nose homogenates were clarified by centrifugation and diluted in Eagle’s Minimum Essential Medium (EMEM). Confluent Hep-2 cell monolayers were infected in duplicate with diluted homogenates in 24 well plates. After a one-hour incubation at 37° C. in a 5% CO₂ incubator, the wells were overlayed with 0.75% methylcellulose medium. After 4 days of incubation, the overlays were removed, and the cells were fixed with 0.1% crystal violet for one hour and then rinsed and air dried. Plaques were counted and virus titer was expressed as plaque forming units per gram of tissue. Viral titers were calculated as geometric mean + standard error for all animals in a group.

An HRSV-A Virospot assay was performed to detect HRSV-specific neutralization antibodies in the sera samples as described in Example 3.

RSV-F enzyme-linked immunosorbent assays (ELISAs) were performed to determine the antibody titer present in the animals’ sera. Briefly, 96-well microtiter plates were coated with 1ug/mL of prefusion RSV-F protein. After an overnight incubation at 4° C. plates were washed 4 times with PBS/0.05% Tween-20 and blocked for 2 hours at 37° C. (SuperBlock- Pierce #37515). After washing, serial dilutions of cotton rat serum were added (assay diluent was PBS + 5% goat serum). Plates were incubated for 2 hours at 37° C., washed and HRP-conjugated chicken anticotton rat IgG (ICL #CCOT-25P) added at a 1:10,000 dilution in assay diluent. Plates were incubated for one hour at 37° C., and then washed. Bound antibody was detected with TMB substrate (SeraCare #5120-0077). The reaction was stopped by adding TMB stop solution (SeraCare #5150-0021) and the absorbance was measured at OD_{450 nm}. Titers were determined using a four-parameter logistic curve fit in GraphPad Prism and defined as the reciprocal dilution at approximately OD_{450 nm} = 1.0.

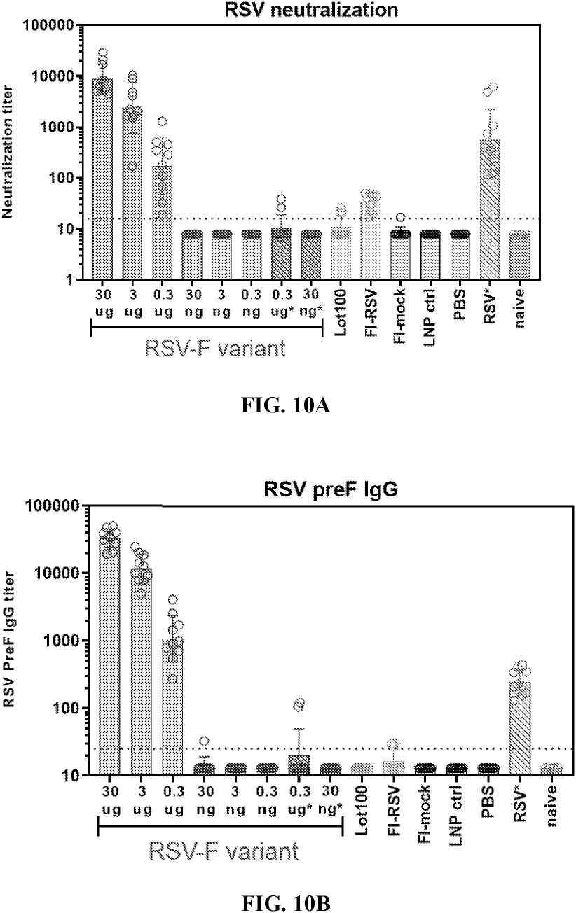

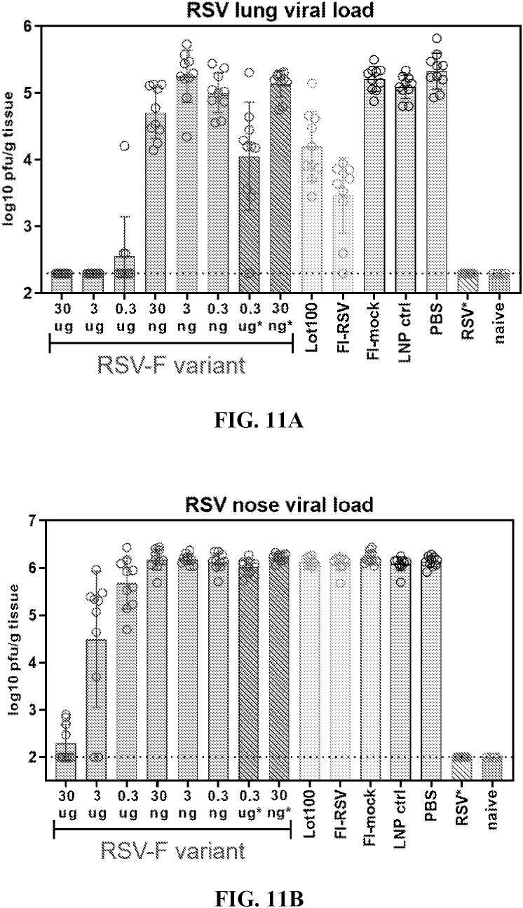

The RSV antibody titers are shown in FIGS. 11A-11B. The RSV F variant (codon-optimized and truncated cytoplasmic tail) was found to induce dose-dependent RSV neutralizing antibodies (FIG. 10A) and RSV prefusion F protein-specific IgG binding antibodies (FIG. 10B). Further, the lung (FIG. 11A) and nose (FIG. 11B) viral loads after challenge demonstrated that the RSV F variant protected the cotton rats from the challenge (in particular, at higher doses with prime and booster doses).

### SEQUENCE LISTING

### Wild-Type RSV F Glycoprotein

MELLIHRSSAIFLTLAINTLYLTSSQNITEEFYQSTCSAVSRGYFSALRT
GWYTSVITIELSNIKETKCNGTDTKVKLIKQELDKYKNAVTELQLLMQNT
PAANNRARREAPQYMNYTINTTKNLNVSISKKRKRRFLGFLLGVGSAIAS
GIAVSKVLHLEGEVNKIKNALLSTNKAVVSLSNGVSVLTSKVLDLKNYIN
NQLLPIVNQQSCRISNIETVIEFQQKNSRLLEITREFSVNAGVTTPLSTY
MLTNSELLSLINDMPITNDQKKLMSSNVQIVRQQSYSIMSIIKEEVLAYV
VQLPIYGVIDTPCWKLHTSPLCTTNIKEGSNICLTRTDRGWYCDNAGSVS
FFPQADTCKVQSNRVFCDTMNSLTLPSEVSLCNTDIFNSKYDCKIMTSKT
DISSSVITSLGAIVSCYGKTKCTASNKNRGIIKTFSNGCDYVSNKGVDTV
SVGNTLYYVNKLEGKNLYVKGEPIINYYDPLVFPSDEFDASISQVNEKIN
QSLAFIRRSDELLHNVNTGKSTTNIMITAIIIVIIVVLLSLIAIGLLLYC
KAKNTPVTLSKDQLSGINNIAFSK (SEQ ID NO: 1)

It should be understood that any of the mRNA sequences described herein may include a 5′ UTR and/or a 3′ UTR. The UTR sequences may be selected from the following sequences, or other known UTR sequences may be used. It should also be understood that any of the mRNAs described herein may further comprise a poly(A) tail and/or cap (e.g., 7mG(5′)ppp(5′)NlmpNp). Further, while many of the mRNAs and encoded antigen sequences described herein include a signal peptide and/or a peptide tag (e.g., C-terminal His tag), it should be understood that the indicated signal peptide and/or peptide tag may be substituted for a different signal peptide and/or peptide tag, or the signal peptide and/or peptide tag may be omitted.

It should be further understood that any of the mRNA sequences described herein may be fully or partially chemically modified (e.g., by N1-methylpseudouridine). In Table 1 below, the sequences numbers are given as unmodified/fully modified by N1-methylpseudouridine).

5′ UTR: GGGAAAUAAGAGAGAAAAGAAGAGUAAGAAGAAAUAUAAGAG
CCACC (SEQ ID NO: 2; fullymodified by N1-methylpse
udoruidine, SEQ ID NO: 33)

5′UTR:GGGAAAUAAGAGAGAAAAGAAGAGUAAGAAGAAAUAUAAGACCC
CGGCGCCGCCACC (SEQID NO: 3; fully modified by N1-m
ethylpseudoruidine, SEQ ID NO: 40)

3′ UTR: UGAUAAUAGGCUGGAGCCUCGGUGGCCAUGCUUCUUGCCCCU
UGGGCCUCCCCCCAGCCCCUCCUCCCCUUCCUGCACCCGUACCCCCGUGG
UCUUUGAAUAAAGUCUGAGUGGGCGGC (SEQ IDNO: 4; fully mo
dified by N1-methylpseudoruidine, SEQ ID NO: 35)

3′ UTR: UGAUAAUAGGCUGGAGCCUCGGUGGCCUAGCUUCUUGCCCCU
UGGGCCUCCCCCCAGCCCCUCCUCCCCUUCCUGCACCCGUACCCCCGUGG
UCUUUGAAUAAAGUCUGAGUGGGCGGC (SEQ IDNO: 5; fully mo
dified by N1-methylpseudoruidine, SEQ ID NO: 41)

TABLE 1

Prefusion RSV F Glycoprotein dCT Variant

| SEQ ID NO: 15 consists of from 5′ end to 3′ end: 5′ UTR SEQ ID NO: 2, mRNA ORF SEQ ID NO: 7, and 3′ UTR SEQ ID NO: 4. | | 15/32 |
|---|---|---|
| SEQ ID NO: 32 consists of from 5′ end to 3′ end: 5′ UTR SEQ ID NO: 33 (SEQ ID NO: 2, fully modified by N1-methylpseudoruidine), mRNA ORF SEQ ID NO: 34 (SEQ ID NO: 7, fully modified by N1-methylpseudoruidine), and 3′ UTR SEQ ID NO: 35 (SEQ ID NO: 4, fully modified by N1-methylpseudoruidine). | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUA AGAGCCACC | 2/33 |
| ORF of mRNA (excluding the stop codon) | AUGGAGCUGCUGAUCCUGAAGGCCAACGCCAUCACGACC AUCCUGACCGCCGUGACCUUCUGCUUCGCCAGCGGGCAGA ACAUCACCGAGGAGUUCUACCAGUCCACCUGCUCCGCCGU GAGCAAGGGCUACCUGUCUGCCCUGAGAACCGGCUGGUA CACCAGCGUGAUCACCAUCGAGCUGUCCAACAUCAAGGA GAACAAGUGCAACGGCACCGACGCCAAGGUGAAGCUGAU CAAGCAGGAGCUGGACAAGUACAAGAACGCAGUGACCGA GCUGCAGCUGCUGAUGCAGAGCACACCAGCCACCGGUAG CGGGUCCGCCAUUUGCUCCGGCGUGGCCGUGUGCAAGGU GCUGCACCUGGAGGGCGAGGUGAACAAGAUCAAGAGCGC CCUGCUCUCCACCAACAAGGCCGUGGUGAGCCUGAGCAAC GGGGUGAGCGUGCUGACCUUCAAGGUGCUGGACCUGAAG AACUACAUCGACAAGCAGCUGCUGCCUAUCCUGAACAAG CAGAGCUGCAGCAUCAGCAACAUCGAGACCGUGAUCGAG UUCCAGCAGAAGAACAACCGGCUGCUGGAGAUCACCAGG GAGUUCAGCGUGAACGCAGGGGUGACCACACCCGUGUCC ACCUACAUGCUGACCAACUCCGAGCUGCUGAGCCUGAUC AACGAUAUGCCCAUCACCAACGACCAGAAGAAGCUGAUG AGCAACAACGUGCAGAUCGUGCGGCAGCAGUCCUACUCC AUCAUGUGCAUCAUCAAGGAGGAGGUGCUGGCCUACGUG GUGCAGCUGCCCCUGUACGGCGUGAUCGACACCCCUUGCU GGAAGCUGCACACCAGCCCUCUGUGCACCACCAACACGAA GGAGGGCAGCAAUAUCUGCCUGACCCGGACCGACAGGGG CUGGUACUGCGACAACGCCGGCAGCGUGUCCUUCUUUCCC CAGGCCGAGACCUGCAAGGUGCAGUCCAACAGGGUGUUC UGCGACACCAUGAACUCUCGCACCCUGCCCAGCGAGGUGA ACCUGUGCAACGUGGACAUCUUCAACCCCAAGUACGACU GCAAGAUCAUGACCUCCAAGACCGACGUGUCCUCUAGCG UUAUCACCUCCCUGGGCGCCAUCGUGAGCUGCUACGGCA AGACCAAGUGCACCGCCAGCAACAAGAACAGGGGCAUCA UCAAGACCUUCAGCAACGGGUGCGACUACGUGUCCAACA AGGGCGUGGACACCGUGUCCGUGGGCAACACCCUGUACU GCGUGAACAAGCAGGAGGGCAAGAGCCUGUACGUGAAGG GCGAGCCCAUCAUCAACUUCUACGACCCUCUGGUGUUCCC CAGCGACGAGUUCGACGCCAGCAUCUCCCAGGUGAACGAGAAGAUCAACCAGAGCCUGGCCUUCAUCCGCAAGAGCGA CGAGCUGCUGCACAACGUGAACGCCGGCAAGAGCACCAC AAACAUCAUGAUCACCACCAUCAUCAUCGUGAUAAUCGU GAUCCUGCUGUCCCUGAUCGCUGUGGGCCUGCUGCUGUA C | 7/34 |
| 3′ UTR | UGAUAAUAGGCUGGAGCCUCGGUGGCCAUGCUUCUUGCC CCUUGGGCCUCCCCCCAGCCCCUCCUCCCCUUCCUGCACC CGUACCCCCGUGGUCUUUGAAUAAAGUCUGAGUGGGCGG C | 4/35 |
| Corresponding amino acid sequence | MELLILKANAITTILTAVTFCFASGQNITEEFYQSTCSAVSKGY LSALRTGWYTSVITIELSNIKENKCNGTDAKVKLIKQELDKYK NAVTELQLLMQSTPATGSGSAICSGVAVCKVLHLEGEVNKIK SALLSTNKA VVSLSNGVSVLTFKVLDLKNYIDKQLLPILNKQS CSISNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSRTLPSEVNLCNVDIFNPKYDCKIMTSKTDVSSSVITSLGAIVSCYGKTKCTASNKNRGIIKTFSNGCDYVSNKGVDTVSVGNTLYCVNKQEGKSLYVKGEPIINFYDPLVFPSDEFDASISQVNEKINQSLAFIRKSDELLHNVNAGKSTTNIMITTIIIVIIVILLSLIAVGLLLY | 8 |
| PolyA tail | 100 nt | |
| hMPV F Glycoprotein | | |
| SEQ ID NO: 16 consists of from 5′ end to 3′ end: 5′ UTR SEQ ID NO: 2, mRNA ORF SEQ ID NO: 10, and 3′ UTR SEQ ID NO: 4. | | 16/36 |
| SEQ ID NO: 36 consists of from 5′ end to 3′ end: 5′ UTR SEQ ID NO: 33 (SEQ ID NO: 2, fully modified by N1-methylpseudoruidine), mRNA ORF SEQ ID NO: 37 (SEQ ID NO: 10, fully modified by N1-methylpseudoruidine), and 3′ UTR SEQ ID NO: 35 (SEQ ID NO: 4, fully modified by N1-methylpseudoruidine). | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUA AGAGCCACC | 2/33 |
| ORF of mRNA (excluding the stop codon) | AUGAGCUGGAAGGUGGUGAUUAUCUUCAGCCUGCUGAUU ACACCUCAACACGGCCUGAAGGAGAGCUACCUGGAAGAG AGCUGCUCCACCAUCACCGAGGGCUACCUGAGCGUGCUGC GGACCGGCUGGUACACCAACGUGUUCACCCUGGAGGUGG GCGACGUGGAGAACCUGACCUGCAGCGACGGCCCUAGCC UGAUCAAGACCGAGCUGGACCUGACCAAGAGCGCUCUGA GAGAGCUGAAGACCGUGUCCGCCGACCAGCUGGCCAGAG AGGAACAGAUCGAGAACCCUCGGCAGAGCAGAUUCGUGC UGGGCGCCAUCGCUCUGGGAGUCGCCGCUGCCGCUGCAG UGACAGCUGGAGUGGCCAUUGCUAAGACCAUCAGACUGG AAAGCGAGGUGACAGCCAUCAACAAUGCCCUGAAGAAGA CCAACGAGGCCGUGAGCACCCUGGGCAAUGGAGUGAGAG UGCUGGCCACAGCCGUGCGGGAGCUGAAGGACUUCGUGA GCAAGAACCUGACCAGAGCCAUCAACAAGAACAAGUGCG ACAUCGAUGACCUGAAGAUGGCCGUGAGCUUCUCCCAGU UCAACAGACGGUUCCUGAACGUGGUGAGACAGUUCUCCG ACAACGCUGGAAUCACACCUGCCAUUAGCCUGGACCUGA UGACCGACGCCGAGCUGGCUAGAGCCGUGCCCAACAUGCC CACCAGCGCUGGCCAGAUCAAGCUGAUGCUGGAGAACAG AGCCAUGGUGCGGAGAAAGGGCUUCGGCAUCCUGAUUGG GGUGUAUGGAAGCUCCGUGAUCUACAUGGUGCAGCUGCC CAUCUUCGGCGUGAUCGACACACCCUGCUGGAUCGUGAA GGCCGCUCCUAGCUGCUCCGAGAAGAAAGGAAACUAUGC CUGUCUGCUGAGAGAGGACCAGGGCUGGUACUGCCAGAA CGCCGGAAGCACAGUGUACUAUCCCAACGAGAAGGACUG CGAGACCAGAGGCGACCACGUGUUCUGCGACACCGCUGCCGGAAUCAACGUGGCCGAGCAGAGCAAGGAGUGCAACAUC AACAUCAGCACAACCAACUACCCCUGCAAGGUGAGCACCG GACGGCACCCCAUCAGCAUGGUGGCUCUGAGCCCUCUGG GCGCUCUGGUGGCCUGCUAUAAGGGCGUGUCCUGUAGCA UCGGCAGCAAUCGGGUGGGCAUCAUCAAGCAGCUGAACA AGGGAUGCUCCUACAUCACCAACCAGGACGCCGACACCGU GACCAUCGACAACACCGUGUACCAGCUGAGCAAGGUGGA GGGCGAGCAGCACGUGAUCAAGGGCAGACCCGUGAGCUC CAGCUUCGACCCCAUCAAGUUCCCUGAGGACCAGUUCAAC GUGGCCCUGGACCAGGUGUUUGAGAACAUCGAGAACAGC CAGGCCCUGGUGGACCAGAGCAACAGAAUCCUGUCCAGC GCUGAGAAGGGCAACACCGGCUUCAUCAUUGUGAUCAUU CUGAUCGCCGUGCUGGGCAGCUCCAUGAUCCUGGUGAGC AUCUUCAUCAUUAUCAAGAAGACCAAGAAACCCACCGGA GCCCCUCCUGAGCUGAGCGGCGUGACCAACAAUGGCUUC AUUCCCCACAACUGA | 10/37 |
| 3′ UTR | UGAUAAUAGGCUGGAGCCUCGGUGGCCAUGCUUCUUGCC CCUUGGGCCUCCCCCCAGCCCCUCCUCCCCUUCCUGCACC CGUACCCCCGUGGUCUUUGAAUAAAGUCUGAGUGGGCGG C | 4/35 |
| Corresponding amino acid sequence | MSWKVVIIFSLLITPQHGLKESYLEESCSTITEGYLSVLRTGWYTNVFTLEVGDVENLTCSDGPSLIKTELDLTKSALRELKTVSAD QLAREEQIENPRQSRFVLGAIALGVAAAAAVTAGVAIAKTIRL ESEVTAINNALKKTNEAVSTLGNGVRVLATAVRELKDFVSKN LTRAINKNKCDIDDLKMAVSFSQFNRRFLNVVRQFSDNAGITP AISLDLMTDAELARAVPNMPTSAGQIKLMLENRAMetVRRKG FGILIGVYGSSVIYMVQLPIFGVIDTPCWIVKAAPSCSEKKGNYACLLREDQGWYCQNAGSTVYYPNEKDCETRGDHVFCDTAA GINVAEQSKECNINISTTNYPCKVSTGRHPISMVALSPLGALVACYKGVSCSIGSNRVGIIKQLNKGCSYITNQDADTVTIDNTVYQLSKVEGEQHVIKGRPVSSSFDPIKFPEDQFNVALDQVFENIENSQALVDQSNRILSSAEKGNTGFIIVIILIAVLGSSMILVSIFIIIKKTKKPTGAPPELSGVTNNGFIPHN | 11 |
| PolyA tail | 100 nt | |
| SEQ ID NO: 17 consists of from 5′ end to 3′ end: 5′ UTR SEQ ID NO: 2, mRNA ORF SEQ ID NO: 13, and 3′ UTR SEQ ID NO: 4. | | 17/38 |
| SEQ ID NO: 38 consists of from 5′ end to 3′ end: 5′ UTR SEQ ID NO: 33 (SEQ ID NO: 2, fully modified by N1-methylpseudoruidine), mRNA ORF SEQ ID NO: 39 (SEQ ID NO: 13, fully modified by N1-methylpseudoruidine), and 3′ UTR SEQ ID NO: 35 (SEQ ID NO: 4, fully modified by N1-methylpseudoruidine). | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUA AGAGCCACC | 2/33 |
| ORF of mRNA (excluding the stop codon) | CGUGCCCUCUAUCGCCCGGCUGGGCUGUGAAGCUGCCGG ACUGCAGCUGGGCAUUGCCCUGACACAGCACUACAGCGA GCUGACCAACAUCUUCGGCGACAACAUCGGCAGCCUGCA GGAAAAGGGCAUUAAGCUGCAGGGAAUCGCCAGCCUGUA CCGCACCAACAUCACCGAGAUCUUCACCACCAGCACCGUG GAUAAGUACGACAUCUACGACCUGCUGUUCACCGAGAGC AUCAAAGUGCGCGUGAUCGACGUGGACCUGAACGACUAC AGCAUCACCCUGCAAGUGCGGCUGCCCCUGCUGACCAGAC UGCUGAACACCCAGAUCUACAAGGUGGACAGCAUCUCCU ACAACAUCCAGAACCGCGAGUGGUACAUCCCUCUGCCCAG CCACAUUAUGACCAAGGGCGCCUUUCUGGGCGGAGCCGA CGUGAAAGAGUGCAUCGAGGCCUUCAGCAGCUACAUCUG CCCCAGCGACCCUGGCUUCGUGCUGAACCACGAGAUGGA AAGCUGCCUGAGCGGCAACAUCAGCCAGUGCCCCAGAACC ACCGUGACCUCCGACAUCGUGCCCAGAUACGCCUUCGUGA AUGGCGGCGUGGUGGCCAACUGCAUCACCACCACCUGUA CCUGCAACGGCAUCGGCAACCGGAUCAACCAGCCUCCCGA UCAGGGCGUGAAGAUUAUCACCCACAAAGAGUGUAACAC CAUCGGCAUCAACGGCAUGCUGUUCAAUACCAACAAAGA GGGCACCCUGGCCUUCUACACCCCCGACGAUAUCACCCUG AACAACUCCGUGGCUCUGGACCCCAUCGACAUCUCCAUCG AGCUGAACAAGGCCAAGAGCGACCUGGAAGAGUCCAAAG AGUGGAUCCGGCGGAGCAACCAGAAGCUGGACUCUAUCG GCAGCUGGCACCAGAGCAGCACCACCAUCAUCGUGAUCCU GAUUAUGAUGAUUAUCCUGUUCAUCAUCAACAUUACCAU CAUCACUAUCGCCAUUAAGUACUACCGGAUCCAGAAACG GAACCGGGUGGACCAGAAUGACAAGCCCUACGUGCUGAC AAACAAG | 13/39 |
| 3′ UTR | UGAUAAUAGGCUGGAGCCUCGGUGGCCAUGCUUCUUGCC CCUUGGGCCUCCCCCCAGCCCCUCCUCCCCUUCCUGCACC CGUACCCCCGUGGUCUUUGAAUAAAGUCUGAGUGGGCGG C | 4/35 |
| Corresponding amino acid sequence | MPISILLIITTMIMASHCQIDITKLQHVGVLVNSPKGMKISQNFETRYLILSLIPKIEDSNSCGDQQIKQYKRLLDRLIIPLYDGLRLQKDVIVTNQESNENTDPRTERFFGGVIGTIALGVATSAQITAAVALVEAKQARSDIEKLKEAIRDTNKAVQSVQSSVGNLIVAIKSVQDYVNKEIVPSIARLGCEAAGLQLGIALTQHYSELTNIFGDNIGSLQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALDPIDISIELNKAKSDLEESKEWIRRSNQKLDSIGSWHQSSTTIIVILIMMIILFIINITIITIAIKYYRIQKRNRVDQNDKPYVLTNK | 14 |
| PolyA tail | 100 nt | |

* It should be understood that any one of the open reading frames and/or corresponding amino acid sequences described in Table 1 may include or exclude a signal sequence. It should also be understood that the signal sequence may be replaced by a different signal sequence, for example, any one of SEQ ID NOs: 18-34.

### EQUIVALENTS

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.” It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

The terms “about” and “substantially” preceding a numerical value mean ±10% of the recited numerical value.

Where a range of values is provided, each value between and including the upper and lower ends of the range are specifically contemplated and described herein.

The entire contents of International Application Nos. PCT/US2015/02740, PCT/US2016/043348, PCT/US2016/043332, PCT/US2016/058327, PCT/US2016/058324, PCT/US2016/058314, PCT/US2016/058310, PCT/US2016/058321, PCT/US2016/058297, PCT/US2016/058319, and PCT/US2016/058314 are incorporated herein by reference.

What is claimed is:

1. A composition comprising a human respiratory syncytial virus (hRSV) ribonucleic acid (RNA) encoding a stabilized prefusion form of an hRSV F glycoprotein variant that lacks a cytoplasmic tail and has at least 90% identity to a wild-type hRSV F glycoprotein.

2. The composition of claim 1, wherein the cytoplasmic tail comprises the C-terminal 20-30, 20-25, 15-30, 15-25, 15-20, 10-30, 10-25, 10-20, 10-15, 5-30, 5-25, 5-20, or 5-15 amino acids of the of the hRSV F glycoprotein variant.

3. The composition of claim 2, wherein the cytoplasmic tail comprises the C-terminal 25 amino acids, 20 amino acids, 15 amino acids, or 10 amino acids of the hRSV F glycoprotein variant.

4. The composition of claim 3, wherein the cytoplasmic tail comprises of the following C-terminal amino acids of the hRSV F glycoprotein variant:
CKARSTPVTLSKDQLSGINNIAFSN (SEQ ID NO: 25); TPVTLSKDQLSGINNIAFSN (SEQ ID NO: 26); SKDQLSGINNIAFSN (SEQ ID NO: 27); or SGINNIAFSN (SEQ ID NO: 28).

5. The composition of any one of claims 1-4, wherein the hRSV F glycoprotein variant comprises a modification, relative to a wild-type hRSV F glycoprotein, selected from the group consisting of: a P102X substitution, a substitution of amino acids 104-144 with a linker molecule, an A149X substitution, an S155X substitution, an S190X substitution, a V207X substitution, an S290X substitution, a L373X substitution, an I379X substitution, an M447X substitution, and a Y458X substitution, wherein X is any amino acid.

6. The composition of claim 5, wherein the hRSV F glycoprotein variant comprises a modification, relative to a wild-type hRSV F glycoprotein, selected from the group consisting of: a P102A substitution, a substitution of amino acids 104-144 with a linker molecule, an A149C substitution, an S155C substitution, an S190F substitution, a V207L substitution, an S290C substitution, a L373R substitution, an I379V substitution, an M447V substitution, and a Y458C substitution.

7. The composition of claim 6, wherein the hRSV F glycoprotein variant comprises the following modifications, relative to a wild-type hRSV F glycoprotein: a P102A substitution, a substitution of amino acids 104-144 with a linker molecule, an A149C substitution, an S155C substitution, an S190F substitution, a V207L substitution, an S290C substitution, a L373R substitution, an I379V substitution, an M447V substitution, and a Y458C substitution.

8. The composition of any one of the preceding claims, wherein the wild-type hRSV F glycoprotein comprises the sequence of SEQ ID NO: 1.

9. The composition of any one of the preceding claims, wherein the hRSV F glycoprotein variant comprises a sequence that has at least 95% or at least 98% identity to the sequence of SEQ ID NO: 8.

10. The composition of claim 9, wherein the hRSV F glycoprotein variant comprises the sequence of SEQ ID NO: 8.

11. The composition of any one of the preceding claims, wherein the hRSV RNA comprises an open reading frame (ORF) that comprises a sequence that has at least 90%, at least 95%, or at least 98% identity to the sequence of SEQ ID NO: 7.

12. The composition of claim 11, wherein the hRSV RNA comprises an ORF that comprises the sequence of SEQ ID NO: 7.

13. The composition of claim 11 or 12, wherein the hRSV RNA comprises a 5′ untranslated region (UTR) that comprises the sequence of SEQ ID NO: 2.

14. The composition of any one of claims 11-13, wherein the hRSV RNA comprises a 3′ UTR that comprises the sequence of SEQ ID NO: 4.

15. The composition of any one of claims 11-14, wherein the hRSV RNA comprises a sequence that has at least 90%, at least 95%, or at least 98% identity to the sequence of SEQ ID NO: 15.

16. The composition of claim 15, wherein the hRSV RNA comprises the sequence of SEQ ID NO: 15.

17. The composition of any one of the preceding claims, wherein the hRSV RNA further comprises a 7mG(5′)ppp(5′)NlmpNp cap.

18. The composition of any one of the preceding claims, wherein the hRSV RNA further comprises a poly(A) tail, optionally having a length of 50 to 150 nucleotides.

19. The composition of any one of the preceding claims, wherein the hRSV RNA comprises a chemical modification.

20. The composition of claim 19, wherein the hRSV RNA is fully modified.

21. The composition of claim 19 or claim 20, wherein the chemical modification is 1-methylpseudouridine.

22. The composition of any one of the preceding claims, comprising 25 µg - 200 µg of the hRSV RNA.

23. The composition of any one of the preceding claims, further comprising a mixture of lipids that comprises a PEG-modified lipid, a non-cationic lipid, a sterol, an ionizable cationic lipid, or any combination thereof.

24. The composition of claim 23, wherein the mixture of lipids comprises 0.5-15 mol% PEG-modified lipid; 5-25 mol% non-cationic lipid; 25-55 mol% sterol; and 20-60 mol% ionizable cationic lipid.

25. The composition of claim 24, wherein the mixture of lipids comprises 1-5 mol% PEG-modified lipid; 10-20 mol% non-cationic lipid; 35-45 mol% sterol; and 40-50 mol% ionizable cationic lipid.

26. The composition of claim 24 or claim 25, wherein the PEG-modified lipid is 1,2 dimyristoyl-sn-glycerol, methoxypolyethyleneglycol (PEG2000 DMG), the non-cationic lipid is 1,2 distearoyl-sn-glycero-3-phosphocholine (DSPC), the sterol is cholesterol; and the ionizable cationic lipid has the structure of Compound 1: 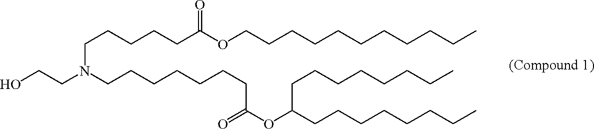 [SMILES: CCCCCCCCCCCOC(=O)CCCCCN(CCO)CCCCCCCC(=O)OC(CCCCCCCC)CCCCCCCC]
.

27. The composition of any one of claims 23-26, wherein the mixture of lipids forms lipid nanoparticles.

28. The composition of claim 27, wherein the hRSV RNA is formulated in the lipid nanoparticles.

29. The composition of any one of the preceding claims, wherein at least 24 hours post-delivery of the composition to mammalian cells in vitro results in cell surface expression of the prefusion form of hRSV F glycoprotein at a level that is at least 5-fold higher than a control level.

30. The composition of any one of the preceding claims, wherein at least 48 hours post-delivery of the composition to mammalian cells in vitro results in cell surface expression of the stabilized prefusion form of hRSV F glycoprotein at a level that is at least 50-fold higher than a control level.

31. A method comprising administering to a subject the composition of any one of claims 1-30 in an amount effective to induce a neutralizing antibody response against hRSV in the subject.

32. The method of claim 31, wherein the subject is immunocompromised.

33. The method of claim 31 or 32, wherein the subject has a pulmonary disease.

34. The method of any one of claims 31-33, wherein the subject is 5 years of age or younger.

35. The method of any one of claims 31-33, wherein the subject is 65 years of age or older.

36. The method of any one of claims 31-35, comprising administering to the subject at least two doses of the composition.

37. The method of any one of claims 31-36, wherein the hRSV F glycoprotein variant folds into a stabilized prefusion conformation.

38. The method of any one of claims 31-37, wherein at least 24 hours post-administration the hRSV F glycoprotein variant is expressed in the subject at a level that is at least 5-fold higher, relative to a control.

39. The method of any one of claims 31-38, wherein at least 48 hours post-administration the RSV F glycoprotein variant is expressed in the subject at a level that is at least 50-fold higher, relative to a control.

40. The method of any one of claims 31-39, wherein the RSV F glycoprotein variant is expressed in the subject for at least 24 hours longer, relative to a control.

41. The method of any one of claims 31-40, wherein a neutralizing antibody response is induced in the subject using a dose of the composition that is at least 5-fold lower, relative to a control.

42. The method of any one of claims 31-42, wherein the control is baseline, administration of an RSV RNA encoding a wild-type RSV F glycoprotein, or administration of an RSV RNA encoding a stabilized prefusion form of an RSV F glycoprotein with a cytoplasmic tail.

43. A messenger ribonucleic acid (mRNA) comprising an open reading frame that comprises a sequence having at least 85%, at least 90%, at least 95%, or at least 98% identity to the sequence of SEQ ID NO: 7.

44. A messenger ribonucleic acid (mRNA) comprising a sequence having at least 85%, at least 90%, at least 95%, or at least 98% identity to the sequence of SEQ ID NO: 15.

45. The mRNA of claim 43 or 44, wherein the mRNA encodes a stabilized prefusion form of an human respiratory syncytial virus (hRSV) ribonucleic F glycoprotein variant that lacks a cytoplasmic tail, wherein the RSV F glycoprotein variant has at least 85%, at least 90%, at least 95%, or at least 98% identity to a full-length wild-type RSV F glycoprotein.

46. The mRNA of any one of claims 43-45, wherein the open reading from comprises the sequence of SEQ ID NO: 7.

47. The mRNA of any one of claims 43-46, wherein the mRNA comprises the sequence of SEQ ID NO: 15.

48. The mRNA of any one of claims 43-47 formulated in a lipid nanoparticle.

49. The mRNA of any one of claims 43-48, wherein the lipid nanoparticle comprises 0.5-15% PEG-modified lipid; 5-25% non-cationic lipid; 25-55% sterol; and 20-60% ionizable cationic lipid.

50. The mRNA of claim 49, wherein the lipid nanoparticle comprises 1-5 mol% PEG-modified lipid; 10-20 mol% non-cationic lipid; 35-45 mol% sterol; and 40-50 mol% ionizable cationic lipid.

51. The mRNA of any one of claims 43-50, wherein the PEG-modified lipid is 1,2 dimyristoyl-sn-glycerol, methoxypolyethyleneglycol (PEG2000 DMG), the non-cationic lipid is 1,2 distearoyl-sn-glycero-3-phosphocholine (DSPC), the sterol is cholesterol; and the ionizable cationic lipid has the structure of Compound 1: 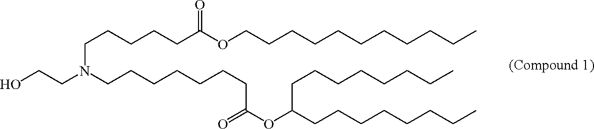 [SMILES: CCCCCCCCCCCOC(=O)CCCCCN(CCO)CCCCCCCC(=O)OC(CCCCCCCC)CCCCCCCC]
.

52. A composition comprising:
a human respiratory syncytial virus (hRSV) ribonucleic acid (RNA) encoding a stabilized prefusion form of an RSV F glycoprotein variant that lacks a cytoplasmic tail, wherein the RSV F glycoprotein variant has at least 85% identity to a full-length wild-type RSV F glycoprotein;
a human metapneumovirus (hMPV) RNA encoding an hMPV F glycoprotein; and
a human parainfluenza virus 3 (hPIV3) RNA encoding an hPIV3 F glycoprotein.

53. The composition of claim 52, wherein the cytoplasmic tail comprises the C-terminal 20-30, 20-25, 15-30, 15-25, 15-20, 10-30, 10-25, 10-20, 10-15, 5-30, 5-25, 5-20, or 5-15 amino acids of the of the hRSV F glycoprotein variant.

54. The composition of claim 53, wherein the cytoplasmic tail comprises the C-terminal 25 amino acids, 20 amino acids, 15 amino acids, or 10 amino acids of the hRSV F glycoprotein variant.

55. The composition of claim 54, wherein the cytoplasmic tail comprises of the following C-terminal amino acids of the hRSV F glycoprotein variant:
CKARSTPVTLSKDQLSGINNIAFSN (SEQ ID NO: 25); TPVTLSKDQLSGINNIAFSN (SEQ ID NO: 26); SKDQLSGINNIAFSN (SEQ ID NO: 27); or SGINNIAFSN (SEQ ID NO: 28).

56. The composition of any one of claims 52-55, wherein the hRSV F glycoprotein variant further comprises a modification, relative to the wild-type hRSV F glycoprotein, selected from the group consisting of: a P102X substitution, a substitution of amino acids 104-144 with a linker molecule, an A149X substitution, an S155X substitution, an S190X substitution, a V207X substitution, an S290X substitution, a L373X substitution, an I379X substitution, an M447X substitution, and a Y458X substitution, wherein X is any amino acid.

57. The composition of claim 56, wherein the hRSV F glycoprotein variant further comprises a modification, relative to the wild-type hRSV F glycoprotein, selected from the group consisting of: a P102A substitution, a substitution of amino acids 104-144 with a linker molecule, an A149C substitution, an S155C substitution, an S190F substitution, a V207L substitution, an S290C substitution, a L373R substitution, an I379V substitution, an M447V substitution, and a Y458C substitution.

58. The composition of claim 57, wherein the hRSV F glycoprotein variant further comprises the following modifications, relative to the wild-type hRSV F glycoprotein: a P102A substitution, a substitution of amino acids 104-144 with a linker molecule, an A149C substitution, an S155C substitution, an S190F substitution, a V207L substitution, an S290C substitution, a L373R substitution, an I379V substitution, an M447V substitution, and a Y458C substitution.

59. The composition of any one of claims 52-58, wherein the wild-type hRSV F glycoprotein comprises the sequence of SEQ ID NO: 1.

60. The composition of any one of claims 52-59, wherein the hRSV F glycoprotein variant comprises a sequence that has at least 95% or at least 98% identity to the sequence of SEQ ID NO: 8.

61. The composition of claim 60, wherein the hRSV F glycoprotein variant comprises the sequence of SEQ ID NO: 8.

62. The composition of any one of claims 52-61, wherein the hRSV RNA comprises an open reading frame (ORF) that comprises a sequence that has at least 90%, at least 95%, or at least 98% identity to the sequence of SEQ ID NO: 7.

63. The composition of claim 62, wherein the hRSV RNA comprises an ORF that comprises the sequence of SEQ ID NO: 7.

64. The composition of claim 62 or 63, wherein the hRSV RNA comprises a 5′ untranslated region (UTR) that comprises the sequence of SEQ ID NO: 2.

65. The composition of any one of claims claim 62-64, wherein the hRSV RNA comprises a 3′ UTR that comprises the sequence of SEQ ID NO: 4.

66. The composition of any one of claims 62-65, wherein the hRSV RNA comprises a sequence that has at least 90%, at least 95%, or at least 98% identity to the sequence of SEQ ID NO: 15.

67. The composition of claim 66, wherein the hRSV RNA comprises the sequence of SEQ ID NO: 15.

68. The composition of any one of claims 52-67, wherein the hMPV F glycoprotein comprises a sequence that has at least 90%, at least 95%, or at least 98% identity to the sequence of SEQ ID NO: 11.

69. The composition of claim 68, wherein the hMPV F glycoprotein comprises the sequence of SEQ ID NO: 11.

70. The composition of any one of claims 52-69, wherein the hMPV RNA comprises an ORF that comprises a sequence that has at least 90%, at least 95%, or at least 98% identity to the sequence of SEQ ID NO: 10.

71. The composition of claim 70, wherein the hMPV RNA comprises an ORF that comprises the sequence of SEQ ID NO: 10.

72. The composition of claim 70 or 71, wherein the hMPV RNA comprises a 5′ UTR that comprises the sequence of SEQ ID NO: 2.

73. The composition of any one of claims 70-72, wherein the hMPV RNA comprises a 3′ UTR that comprises the sequence of SEQ ID NO: 4.

74. The composition of any one of claims claim 70-73, wherein the hMPV RNA comprises a sequence that has at least 90%, at least 95%, or at least 98% identity to the sequence of SEQ ID NO: 16.

75. The composition of claim 74, wherein the hMPV RNA comprises the sequence of SEQ ID NO: 16.

76. The composition of any one of claims 52-75, wherein the hPIV3 F glycoprotein comprises a sequence that has at least 90%, at least 95%, or at least 98% identity to the sequence of SEQ ID NO: 14.

77. The composition of claim 76, wherein the hPIV3 F glycoprotein comprises the sequence of SEQ ID NO: 14.

78. The composition of any one of claims 52-77, wherein the hPIV3 RNA comprises an ORF that comprises a sequence that has at least 90%, at least 95%, or at least 98% identity to the sequence of SEQ ID NO: 13.

79. The composition of claim 78, wherein the hPIV3 RNA comprises an ORF that comprises the sequence of SEQ ID NO: 13.

80. The composition of claim 78 or 79, wherein the hPIV3 RNA comprises a 5′ UTR that comprises the sequence of SEQ ID NO: 2.

81. The composition of any one of claims 78-80, wherein the hPIV3 RNA comprises a 3′ UTR that comprises the sequence of SEQ ID NO: 4.

82. The composition of any one of claims 78-81, wherein the hPIV3 RNA comprises a sequence that has at least 90%, at least 95%, or at least 98% identity to the sequence of SEQ ID NO: 17.

83. The composition of claim 82, wherein the hPIV3 RNA comprises the sequence of SEQ ID NO: 17.

84. The composition of any one of claims 52-83, wherein the hRSV RNA, the hMPV RNA, and/or the hPIV3 RNA further comprises a 7mG(5′)ppp(5′)NlmpNp cap.

85. The composition of any one of claims 52-84, wherein the hRSV RNA, the hMPV RNA, and/or the hPIV3 RNA further comprises a poly(A) tail, optionally having a length of 50 to 150 nucleotides.

86. The composition of any one of claims 52-83, wherein the hRSV RNA, the hMPV RNA, and/or the hPIV3 RNA comprises a chemical modification.

87. The composition of claim 86, wherein the hRSV RNA, the hMPV RNA, and/or the hPIV3 RNA is fully modified.

88. The composition of claim 86 or claim 87, wherein the chemical modification is 1-methylpseudouridine.

89. The composition of any one of claims 52-88, comprising 25 µg - 200 µg of the hRSV RNA, the hMPV RNA, and/or the hPIV3 RNA.

90. The composition of any one of claims 52-89, further comprising a mixture of lipids that comprises a PEG-modified lipid, a non-cationic lipid, a sterol, an ionizable cationic lipid, or any combination thereof.

91. The composition of claim 90, wherein the mixture of lipids comprises 0.5-15% PEG-modified lipid; 5-25% non-cationic lipid; 25-55% sterol; and 20-60% ionizable cationic lipid.

92. The composition of claim 91, wherein the lipid nanoparticle comprises 1-5 mol% PEG-modified lipid; 10-20 mol% non-cationic lipid; 35-45 mol% sterol; and 40-50 mol% ionizable cationic lipid.

93. The composition of claim 91 or claim 92, wherein the PEG-modified lipid is 1,2 dimyristoyl-sn-glycerol, methoxypolyethyleneglycol (PEG2000 DMG), the non-cationic lipid is 1,2 distearoyl-sn-glycero-3-phosphocholine (DSPC), the sterol is cholesterol; and the ionizable cationic lipid has the structure of Compound 1: 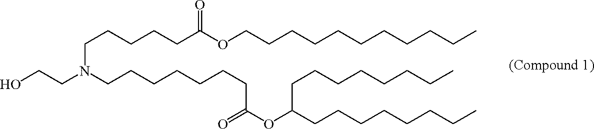 [SMILES: CCCCCCCCCCCOC(=O)CCCCCN(CCO)CCCCCCCC(=O)OC(CCCCCCCC)CCCCCCCC]
.

94. The composition of any one of claims 90-93, wherein the mixture of lipids forms lipid nanoparticles.

95. The composition of claim 94, wherein the hRSV RNA, the hMPV RNA, and the hPIV3 RNA are formulated in the lipid nanoparticles.

96. A method comprising administering to a subject the composition of any one of claims 52-95 in an amount effective to induce a neutralizing antibody response against hRSV, hMPV, and/or hPIV3 in the subject.

97. The method of claim 96, wherein the subject is immunocompromised.

98. The method of claim 96 or 97, wherein the subject has a pulmonary disease.

99. The method of any one of claims 96-98, wherein the subject is 5 years of age or younger.

100. The method of any one of claims 96-98, wherein the subject is 65 years of age or older.

101. The method of any one of claims 96-100, comprising administering to the subject at least two doses of the composition.
